(19) United States
(12) Patent Application Publication
(10) Pub. No.: US 2007/0098643 A1
(43) Pub. Date: May 3, 2007

(54) Oligomers of gadolinium chelates, their applicationascontrast products in magnetic resonance imaging and their synthesis intermediates

(76) Inventors: Isabelle Nachman, Courbevoie (FR); Marc Port, Deuil La Barre (FR); Isabelle Raynal, St-Maur Des Fosses (FR); Olivier Rousseaux, Senlis (FR)

Correspondence Address: JACOBSON HOLMAN PLLC, 400 SEVENTH STREET N.W., SUITE 600, WASHINGTON, DC, 20004, US

(21) Appl. No.: 10/505,875

(22) PCT Filed: Mar. 5, 2003
(86) PCT No.: PCT/FR03/00712

§ 371 Date: Jan. 20, 2006

(30) Foreign Application Priority Data
Mar. 5, 2002 (FR) 02/02791

(51) Int. Cl. A61K 49/10; C07F 5/00

(52) U.S. Cl. 424/9.363; 534/15

(57) ABSTRACT

The invention relates to high-relaxivity macrocyclic gadolinium chelate oligomers of formula
W-(A)ₘ
in which W, A and m are as defined in claim 1, and their use as a blood pool contrast product for magnetic resonance imaging.

The present invention relates to oligomers of paramagnetic chelates, to their application as blood pool contrast products for magnetic resonance imaging (MRI) and to the processes for preparing them and their synthesis intermediates.

The administration to patients of contrast products contributes to improving the resolution of the images obtained and the diagnostic accuracy.

The longitudinal relaxivity r₁ of a paramagnetic contrast product gives a measure of its magnetic efficiency and makes it possible to assess its influence on the signal recorded; the mass efficiency, defined as being the ratio of r₁ to the molecular mass of the given compound, for its part, gives a measure of the efficiency of the weight unit of the contrast product and makes it possible to compare the products in terms of weight of diagnostic dose administered, and in particular of tolerance and cost.

Gadolinium chelates, used clinically in humans, such as Magnevist®, Dotarem® or Omniscan®, have a low molecular weight, have relaxivities r₁ of less than 5 mM⁻¹s⁻¹, and are rapidly distributed in the extravascular space after they have been injected.

Other high-relaxivity products, referred to as RR (restricted rotation) have been described by the Applicant in patent applications WO 97/01359, EP-A-922700 and WO 00/75241. These products are complexes. of paramagnetic gadolinium with nitrogenous macrocycles carrying, on the nitrogen atoms, acetic groups characterized by the presence, on the carbon atom in a position alpha to the carboxyl, of hydrophilic groups.

Certain large-volume gadolinium chelates are products with a prolonged intravascular circulation (Blood Pool Agents or BPA): the prolonged intravascular circulation limits the elimination of the contrast product by the kidneys and by transfer to the extravascular compartments. BPAs are thus aimed at improving the characterization of lesions (for example the vascularization of lesions or the detection of hemorrhages) and the infusion of organs, in particular myocardial perfusion.

Products referred to as RC BPAs (Rapid Clearance Blood Pool Agents) are in particular known. The degree of prolonged intravascular circulation of these RC BPA products makes them advantageous in preferred applications such as myocardial perfusion or coronary artery angiography, where they can be used in several injections successively. Particularly known among the RC PBAs are the products described in document EP 922 700, and products of dendrimer type: several gadolinium chelates are grafted at the periphery of a highly sterically hindered “solid and dense” arborescent structure forming a type of solid sphere (Gadomer17® for example).

That being said, it remains desirable to find products which are even more efficient from a magnetic point of view, the vascular permeability of which is very low, and the elimination of which is even lower than that of the already known RCBPAs. Various attempts have been aimed at obtaining compounds referred to as SC BPAs (Slow Clearance Blood Pool Agent). Among the SC BPAs, products comprising a gadolinium chelate grafted onto a high-molecular weight biological molecule, such as albumin, are in particular known.

That being said, there remains the need for other products which make it possible to optimize the dose of gadolinium, and to improve the quality of the image by means of the selective distribution in the vascular compartment and of improved contrast between the vessels and the surrounding tissues.

In particular, it is desirable to obtain products having a similar, or even greater, efficiency than the dendrimers or the products which bind to albumin, but the chemical synthesis of which is less complex on an industrial level, and the mass efficiency and tolerance of which are improved.

Novel BPAs, with more prolonged intravascular circulation than the known RC BPAs, are aimed at significant improvements in particular in determining the blood volume in the various tissues, differentiating cancerous tumors, identifying inflammatory zones, or lymphography.

The Applicant has now found that polymetallic oligomers, in a star shape around a plurifunctional core called the central nucleus (hence a structure of hollow sphere type instead of solid sphere type), derived from its RR products described above, exhibit significantly improved BPA properties, for weight doses that are acceptable for the patient and a cost that is compatible with health economics. The products are eliminated mainly via the kidneys, slowly, but within a reasonable period of time so as to avoid the appearance of side effects due to prolonged retention in the body, even if the complexes are stable.

These novel oligomers comprise several functionalized gadolinium chelates which have been grafted onto a molecule, the central nucleus, carrying groups capable of reacting with these coupling functions. According to an advantageous embodiment, the coupling functions are amine functions. According to one embodiment, these oligomers are obtained from monomers of chelates, these polymetallics carrying 3 to 6 gadolinium chelates.

According to another embodiment, these oligomers are obtained from dimers of chelates; these polymetallics typically carry from 4 to 8 gadolinium chelates.

They are products which represent a technical solution to several technical problems:

The molecular mass is greater than 10 000 daltons and monodisperse, so as to limit the unwanted extravascular diffusion, which allows better vascular and tissue imaging (characterization imaging);

The proportion by weight of gadolinium ions (Gd content=number of Gd×157.25/molecular mass) is of the order of 4 to 6%, which makes it possible to limit the dose of gadolinium injected (decreased toxicity) and to obtain an improved mass efficiency (mass efficiency per Gd=r1 per Gd×1000/157.25 (molecular mass of Gd)), of greater than 100 g⁻¹Gd s⁻¹.

It is recalled that the molar mass efficiency (me) in g⁻¹s⁻¹ is the ratio: (number of Gd×r1×1000)/molecular mass of the molecule).

In particular, the not very dense star-shaped structure described later makes it possible, for the same desired molecular hindrance (making it possible to reduce the extravascular diffusion), to significantly limit the molecular weight of the compound (and therefore its cost and the difficulty in synthesizing it) compared to a structure of the dendrimer type (proportion by weight of gadolinium ions of the order of 18%, or even much more).

The number of chelates grafted onto the core chosen is completely controlled, unlike the case of the known derivatives which result from the grafting of polymers. Their relaxivity r₁ per Gd is equivalent to or greater than that of the monomers from which they are derived and the signal obtained is. satisfactory within the magnetic field range applied by current medical imaging devices, i.e. typically 20, or even 40 MHz or 60 MHz.

Compared to BPAs of the prior art, the novel products obtained have greater relaxivities per Gd, a greater mass efficiency per Gd (the mass efficiency of the polymetallic oligomer is greater than that of the corresponding monometallic monomer), and a controlled monodispersity and purity.

The known SC BPAs from the prior art suggested:
grafting onto large biological molecules such as proteins, or onto solid chemical carrier structures, to the detriment of a high-performance mass efficiency,
structures of polymer type but much too polydisperse for reliable use, with insufficient purity and homogeneity of the products.

To obtain these novel oligomers, the structure of the RR chelates previously described has had to be modified in order to introduce a group capable of reacting on the central nucleus precursor molecule, without this modification resulting in a loss in stability or magnetic efficiency.

According to a first aspect, the invention relates to polymetallic molecules of general formula below:
CENTRAL NUCLEUS−[(linker 2−Div)ₒ−(linker 1−Gd core−(branch)ₙ)ₛ]ₘ

in which:

n is between 1 and 3

s is between 1 and 3, preferably s=1 or 2

o is 0 or 1

m is between 2 and 6 when o=0, and m is between 1 and 4 when o=1.

The Gd cores carrying hydrophilic branches are gadolinium chelates of RR type, i.e. restricted rotation type, which notion is described in granted patent EP B 661 279 and EP B 922 700, and recalled later. In fact, the inventors have carried out trials with non-RR derivatives, but the results were not satisfactory. The inventors have thus succeeded in constructing efficient products by combining:
the use of restricted rotation Gd derivatives, as described in patents EP B 661 279, EP B 922 700 and EP B 1 183 255, to obtain a satisfactory relaxivity (for a sufficient signal);
the optimized construction of a polymetallic-type architecture allowing a multiplication of the signal per Gd, while at the same time having in particular a molecular volume, a mass efficiency and a hydrophijicity which are sufficient for high-performance use as a BPA, with a final molecule having a high relaxivity for a limited number of Gd.

These novel polymetallic compounds clearly differ from the compounds of dendrimer type (o>1) of the prior art, in which the couples (Div−linker 2) can be different at each generation.

Advantageously, the Gd cores were chosen from:

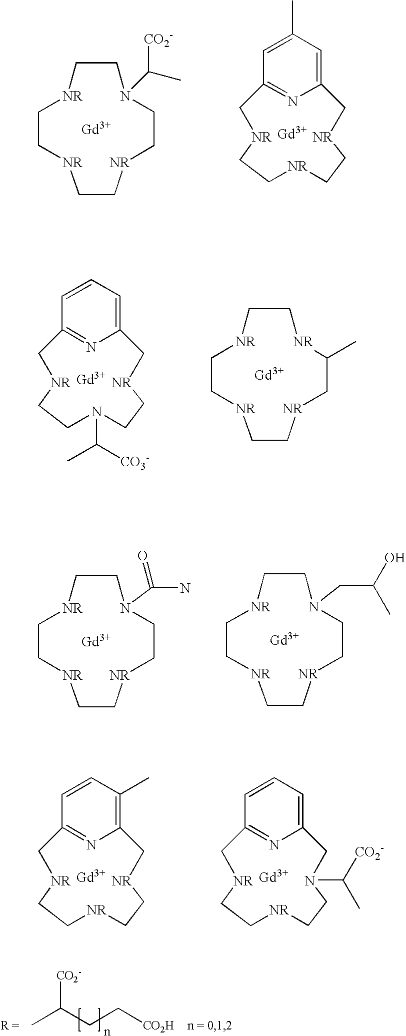

The inventors have also studied compounds using restricted-rotation Gd cores of DTPA type described in particular in document EP 661 279, of general formula: 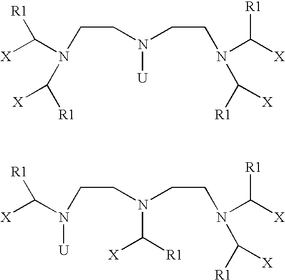
with R1 and U as described later.

Advantageously, the branches were chosen from:
the branches denoted AAG1 AA28Br, AAG1 AA29Br described later;
the branches described in documents EP 661 279, EP 922 700, EP 1 183 255;
the “flash” branches described later;
the “rigid linker” branches CO—NH-φ-CO—NH, referred to as P792, mentioned later.

Such hydrophilic branches forming side arms on the acid groups may be different in nature and are intended to decrease the freedom of movement of the paramagnetic complex and of the paramagnetic ion which is attached thereto, the rotation of which in the magnetic field (inverse function of r1) is thus restricted.

In addition, the inventors have had to, in a nonevident manner, adapt the construction [(Gd core−linker 1)s−(Div−linker 2)ₒ], without this construction resulting in a loss of thermodynamic or kinetic stability or in magnetic efficiency.

When a divider is used, various dividers are possible, as long as they make it possible to provide the link (while preserving the star-shaped structure) between, firstly, at least two chelates and, secondly, a polyfunctional central nucleus. Various polyfunctional backbones can be used as divider by those skilled in the art, described in Chemical Reviews, 2001, 101 (12), 3819-386 and Topics in Current Chemistry, vol 217,212,210,197. Aromatic backbones polyfunctionalized with. carboxylate and/or amino groups are preferred as divider Div.

The Applicant has preferred to use dividers Div of 1,3,5-triazine type:

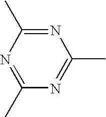

Linker 1 and linker 2 are preferably chosen from:
a) (CH₂)₂-φ-NH₂, (CH₂)₃—NH₂, NH—(CH₂)₂—NH, NH—(CH₂)₃—, NH₂—(CH₂)₂—O(CH₂)₂—O(CH₂)₂—NH₂,
b) P1-I-P2, which may be identical or different, P1 and P2 being chosen from OH, SH, NH₂, H, CO₂H, NCS, NCO, SO₃H,
With I=alkylene (preferably C₁ to C8), cycloalkylene, alkoxyalkylene, polyalkoxyalkylene, alkylene interrupted with phenylene, alkylidene, acylidene.
c) P1-I-P2, with P1 being as in a) or b) and P2 being chosen from hydroxamate, cathecolate, phenanthroline and guanine, when the nucleus is a metal M as described in U.S. Pat. No. 6,056,939 and Topics in current chemistry 2002, 221, 123-164.

The-central NUCLEUS was chosen from two types of groups (the compound in brackets is an example of corresponding compound exemplified in the detailed description):

a) melamine (P799), monochlorinated cyanuryl (MC606 and MC617), terephthalic dithiourea (MC607), phenyltriisothiourea (MC616), P730 Gd, i.e. DOTA Gd with the presence, on the carbon atom in a position alpha to the carboxyl, of hydrophilic groups (MC635), tetrakis- (MC636 and MC645) and hexakis-phosphazene (MC647).

b) polyacid, optionally halogen, core, polyacid or polyamine gadolinium-containing core, isothiocyanate or isocyanate core, polyamine core, polysulfate core, polycarboxylic or polyamino polymer.

c) core (M), chosen from lanthanides such as gadolinium Gd, and transition metals such as Fe, Co, Zn, Ni or Ca2+ when P2 is chosen from hydroxamate, cathecolate, phenanthroline and guanine, as described in US patent U.S. Pat. No. 6,056,939 and Topics in current chemistry 2002, 221, 123-164.

Among all the compounds above, the star-shaped compounds corresponding to general formula (E) below were preferred:
W-(A)m (E)

in which:
W is a central NUCLEUS
A represents [(D)_{q}-(I_{a, b, c, d, e, f})ᵣ];
   (E) being written: W-[(D)_{q}-(I_{a, b, c, d, e, f})ᵣ]ₘ
      With:
q=0 (E is a polymetallic compound of monomers as described later) or q=1 (E is a polymetallic compound of dimers as described later)
r=1 when q=0, or r=2 or 3 when q=1
m is between 3 and 6 when q=0 and m is between 2 and 4 when q=1
D being a polyfunctional molecule capable of linking the central NUCLEUS to at least two metal chelates,
D being written (Div−linker 2), Div being a group having a number of free valencies at least equal to r, Div also being denoted as divider,
D being linked, firstly, to at least two metal chelates by means of linkers 1 (two linkers 1 for the dimers) and, secondly, to the central NUCLEUS by means of a linker 2, linker 1 and linker 2 being as defined above, in the following way for r=2
   (linker 1)₂−Div−(linker 2);
   (E) being written:
      NUCLEUS−[(linker 2−Div)_{q}−(I_{a,b,c,d,e,f})ᵣ]ₘ (E)

Linker 1 being included, as described later, in I_{a,b,c,d,e,f} D preferably being an aromatic backbone polyfunctionalized with carboxylate and/or amino groups, Div preferably being of 1,3,5-triazine type, of formula: 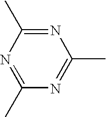
with
(linker 1)₂−Div−(linker 2) being written: 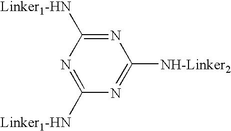
I_{a,b,c,d,e,f} signifies Iₐ or I_{b} or I_{c} or I_{d} or Iₑ or I_{f}, representing the restricted-rotation derivatives
I^{a}, I_{b}, I_{c} having the meanings: 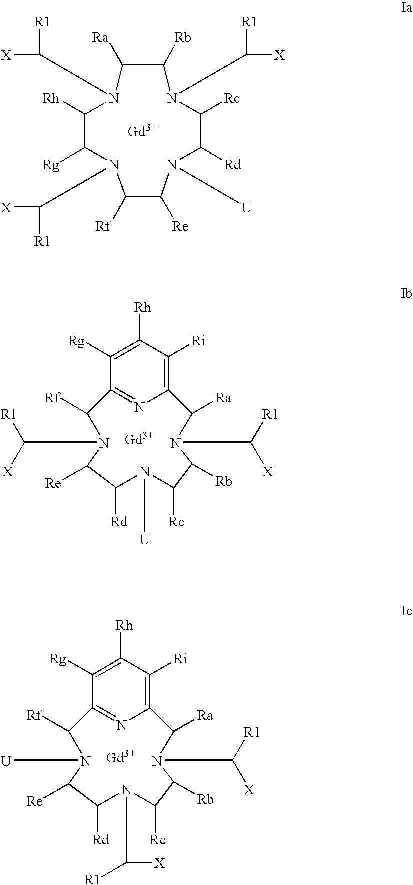
where:
the X, which may be identical or different, are chosen from CO₂R′a, CONR′bR′c or P(R′d)O₂H, with:
   R′a, R′b and R′c, which may be identical or different, being H or (C₁-C₈)-alkyl, optionally hydrnxylated;
   P is the phosphorus atom, R′d represents OH, (C₁-C₈)alkyl or (C₁-C₈)alkoxy, (C₁-C₈)arylalkyl or (C₁-C₈)alkoxyalkyl;
R1 represents a hydrophilic group, of appropriate size for obtaining both a sufficient degree of relaxivity and an effective mass efficiency, typically of molecular mass greater than 300 and less than 3000, comprising at least three oxygen atoms, selected from the following groups:
   polyoxy (C₂-C₃)alkylene (i.e polyoxyethylenes and polyoxypropylenes), in particular polyethylene glycol and C₁ to C₃ monoethers and monoesters thereof, of molecular mass preferably from 1000 to 2000
   polyhydroxyalkyl
   polyol (including functionalized oligosaccharides [this type of functionalization being described in particular in J. Polymer. Sc. Part A Polymer chemistry 23 1395-1405 (1985) and 29, 1271-1279 (1991) and in Bioconjugate chem. 3, 154-159 (1992)])
   (R₂ g)ₑ[(R₂ g)ᵢ R₃]ₕ where:
      h=1 or 2; i=0, 1 or 2; e=1 to 5
      R₂ represents (the R₂ being identical or different):
         nothing, an alkylene, an alkoxyalkylene, a polyalkoxyalkylene;
         a phenylene, or a saturated or unsaturated heterocyclic residue optionally substituted with OH, Cl, Br, I, (C₁-C₈)alkyl, (C₁-C₈)alkyloxy, NO2, NR_{X}R_{Y}, NR_{X}COR_{Y}, CONR_{X}R_{Y} or COOR_{X}, R_{X} and R_{Y} being H or (C₁-C₈)alkyl, and the linear, branched or cyclic C₁ to C₁₄ alkyl, alkylene and alkoxy groups possibly being hydroxylated;
      g represents (the g being identical or different): nothing or a function O, CO, OCO, COO, SO3, OSO2, CONR′, NR′CO, NR′COO, OCONR′, NR′, NR′CS, CSNR′, SO2NR′, NR′SO2, NR′CSO, OCSNR′, NR′CSNR′, P(O)(OH)NR′ or NR′P(O)—(OH), in which R′ is H (C₁-C₈) or R₃;
      R₃ represents alkyl, phenyl, alkyl substituted or interrupted with one or more phenyl groups, or alkyleneoxy; amino or amido, unsubstituted or substituted with alkyl optionally substituted or interrupted with one of the above groups; it being possible for the phenyl, phenylene and heterocyclic groups to be substituted with OH, Cl, Br, I, (C₁-C₈)alkyl, (C₁-C₈)alkyloxy, NO2, NR_{X}R_{Y}, NR_{X}COR_{Y}, CONR_{X}R_{Y} or COOR_{X}, R_{X} and R_{Y} being H or (C₁-C₈)alkyl, and the linear, branched or cyclic C₁ to C₁₄ alkyl, alkylene and alkoxy groups possibly being hydroxylated;
   Rₐ to Rᵢ (i.e. Ra, Rb, Rc, Rd, Re, Rf, Rg, Rh, Ri) independently represent H, alkyl, hydroxyalkyl, alkylphenyl or cycloalkyl.
   U is a group —CX R4-linker 1, CHR4CON-linker1 or CHR4-CHR5OH-linker1
   R4 and R5 independently represent H, alkyl or hydroxyalkyl.
   X and linker 1 having the meaning above.
   I_{d}, Iₑ, I_{f} having the meanings: 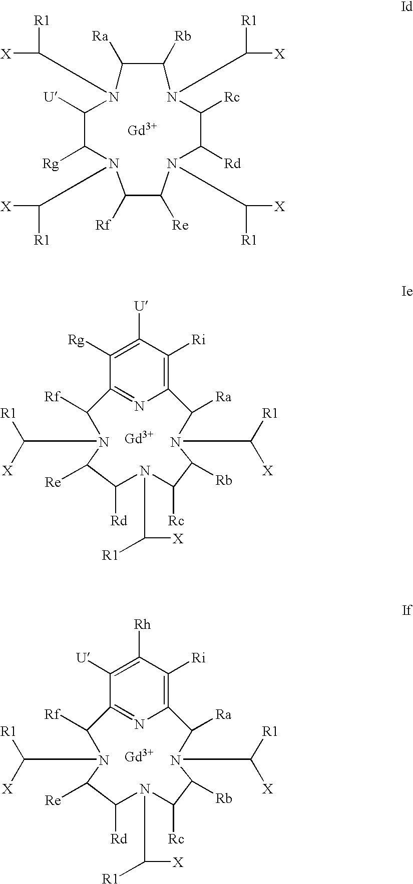
   X, R1, and Ra to Ri having the same meaning as above.
U′ is linker 1.

The central nucleus is chosen from:

a) melamine (P799), monochlorinated cyanuryl (MC606 and MC617), 1-4-phenylene dithiourea (MC607), phenyltriisothiourea (MC616), P730 Gd, i.e. DOTA Gd with the presence, on the carbon atom in a position alpha to the carboxyl, of hydrophilic groups (MC635), tetrakisphenylmethane (MC636 and MC645), hexakis-phosphazene (MC647).

b) polyacid, optionally halogen, core, polyacid or polyamine gadolinium-containing core, isothiocyanate or isocyanate core, polyamine core, polysulfate core, polycarboxylic or polyamino polymer.

c) core (M), chosen from lanthanides such as gadolinium Gd, and transition metals such as Fe, Co, Zn, Ni or Ca2+ when P2 is chosen from hydroxamate, cathecolate, phenanthroline and guanine, as described in U.S. Pat. No. 6,056,939 and Topics in current chemistry 2002, 221, 123-164.

The invention also relates to the salts of the compounds of formula (E) with inorganic or organic acids or bases, in particular the hydrochlorides of the amino groups and the sodium, potassium and N-methylglucamine salts of the carboxylic acid groups present on the chelates.

Moreover, the Applicant makes the following comments:
For U=CXR4-CHR5OH-linker 1 or U=CHR4CON-linker 1, the chemical synthesis is facilitated; however, the relaxivity results are not generally as good with the latter.
Preferably, X represents CO2R′ₐ; however, the use of CONR′b R′c makes it possible to obtain nonionic compounds that are advantageous for decreasing the osmolality of the product, and the use of P(R′_{d})O₂H can make it possible to obtain products with higher relaxivity.
Preferably, Ra, Rb and Rc represent H, but it is also possible to use alkyl or cycloalkyl groups to stabilize the structure and to improve the relaxivity, on condition of not interfering with the desired properties of the product (rigidification by grafting of alkyl groups is known to those skilled in the art in Inorganic Chemistry, vol 41, n^{o}25, p6846-6855, 2002). Hydroxyalkyl groups are known to those skilled in the art to decrease the toxicity of structures, as described in Inorganic Chemical Acta 317, 2001, 218-229, and Coordination Chemistry Reviews, 185-186, 1999, 451-470.

Preference is given to the compounds (E) where R1=(CH2)xCONHR with x=1, 2 or 3 and R is a hydrophilic group of molecular weight greater than 200, chosen from:
1) a group: 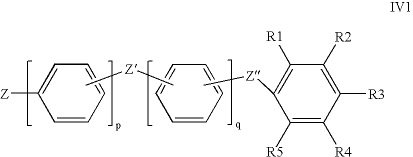
and Z is a bond, CH₂, CH₂CONH or (CH₂)₂NHCO
   Z′ is a bond, O, S, NQ, CH₂, CO, CONQ, NQCO, NQ-CONQ or CONQCH₂CONQ,
   Z″ is a bond, CONQ, NQCO or CONQCH₂CONQ
   p and q are integers the sum of which is 0 to 3;
   R₁, R₂, R₃, R₄ and R₅ represent:
      either, independently of one another, H, Br, Cl, I, CONQ₁Q₂ or NQ₁COQ₂ with Q₁ and Q₂, which may be identical or different, being H or a group (C₁-C₈)alkyl that is mono- or polyhydroxylated or optionally interrupted with one or more oxygen atoms, and at least one and at most two of R₁ to R₅ being CONQ₁Q₂ or NQ₁COQ₂;
      or R₂ and R₄ represent 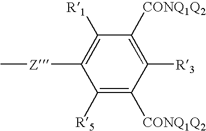
      and R₁, R′₁, R₃, R′₃, R₅ and R′₅, which may be identical or different, represent H, Br, Cl or I, Q₁and Q₂ have the same meaning as above and Z′″ is a group chosen from CONQ, CONQCH₂CONQ, CONQCH₂, NQCONQ and CONQ(CH₂)₂NQCO, and Q is H or optionally hydroxylated (C₁-C₄)alkyl, it being possible for the alkyl groups to be linear or branched;
2) a “flash” branch 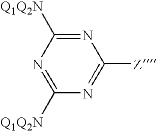
with Z″″ being NQ(CH₂)ⱼ(CH₂OCH₂)ᵢ(CH₂)ⱼNH₂, with i=2 to 6 and j=1 to 6.

Among all the compounds (E) above, two types of compounds are particularly advantageous.

The first type is represented by the compounds called “Polymetallics of Monomers of derivatives with Restricted Rotation”, abbreviated to Poly M RR. These star-shaped, high-relaxivity chelate oligomers have the formula:
W1-(A1)ₘ₁ (I1) Poly M RR
in which:
m1 is 3, 4, 5 or 6;
W1 is the radical of an organic molecule carrying m1 carbonyl groups which form carboxamido groups with A1;
or W1 is a group: 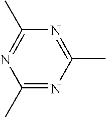
A1 represents the group: 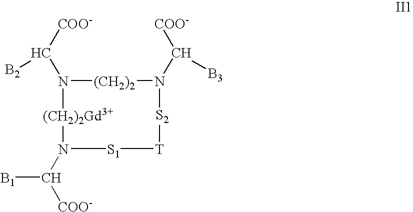
in which:
—S₁-T-S₂— is
   either 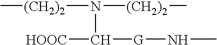
   where S₁=S₂=(CH₂)₂
   with all three of B₁, B₂ and B₃ representing (CH₂)ₓCONHR with x=1, 2 or 3
   or 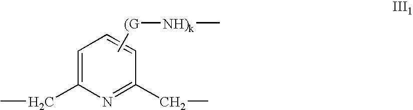
   with k=0 and S₁=S₂=CH₂
   one of B1, B2 andv B3 representing G-NH, and the others representing (CH₂)ₓCONHR
   or 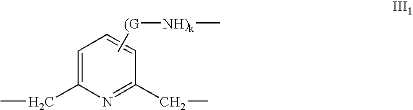
   with k=1
   all three of B₁, B₂ and B₃ representing (CH₂)ₓCONHR with x=1, 2 or 3 and GNH chosen from:
      the groups —(CH₂)ₙ—NH— with n=1 to 4, or 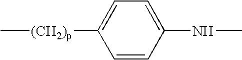
      with p=0 to 3;
it being specified that:
R represents a hydrophilic group of molecular weight greater than 200, R representing, according to one variant, a group: 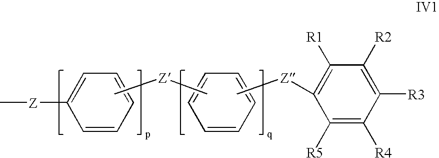
and Z is a bond, CH₂, CH₂CONH or (CH₂)₂NHCO
   Z′ is a bond, O, S, NQ, CH₂, CO, CONQ, NQCO, NQ-CONQ or CONQCH₂CONQ,
   Z″ is a bond, CONQ, NQCO or CONQCH₂CONQ
   p and q are integers the sum of which is 0 to 3;
   R₁, R₂, R₃, R₄ and R₅ represent:
      either, independently of one another, H, Br, Cl, I, CONQ₁Q₂ or NQ₁COQ₂ with Q₁ and Q₂ which may be identical or different, being H or a group (C₁-C₈)alkyl which is mono- or polyhydroxylated or optionally interrupted with one or more oxygen atoms, and at least one and no more than two of R₁ to R₅ being CONQ₁Q₂ or NQ₁COQ₂;
      or R₂ and R₄ represent 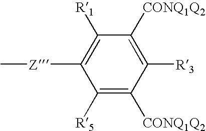
      and R₁, R′₁, R₃, R′₃, R₅ and R′₅, which may be identical or different, represent H, Br, Cl or I, Q₁ and Q₂ have the same meaning as above and Z′″ is a group chosen from CONQ, CONQCH₂CONQ, CONQCH₂, NQCONQ and CONQ(CH₂)₂NQCO, and Q is H or optionally hydroxylated (C₁-C₄)alkyl, it being possible for the alkyl groups to be linear or branched;
R representing, according to another variant (“flash” branches): 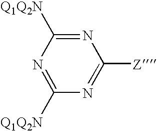
with Z″″ being NQ(CH₂)ⱼ(CH₂OCH₂)ᵢ(CH₂)ⱼNH₂, with i=2 to 6 and j=1 to 6, preferably 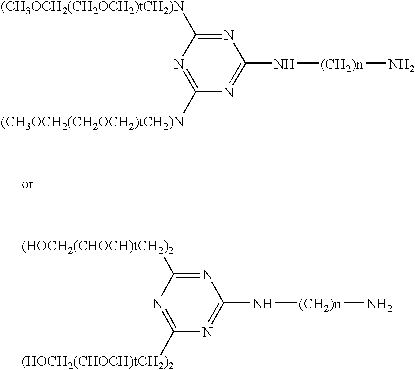
with t=1, 2, 3 or 4 and n=2 to 6.

The invention also relates to the salts of the compounds of formula I1 with inorganic or organic acids or bases, in particular the hydrochlorides of the amino groups and the sodium, potassium and N-methylglucamine salts of the carboxylic acid groups present on the chelates.

The second type is represented by the compounds called “Polymetallics of Dimers of derivatives with restricted rotation”, abbreviated to Poly D RR, of general formula:
W2-(A2)ₘ₂ (I2) Poly D RR
in which:
m2 is 2, 3 or 4;
W2 is the radical of an organic molecule carrying m2 carbonyl groups which 5 form carboxamido groups with A2;
or W2 is a group: 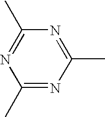
A2 represents 1) the group 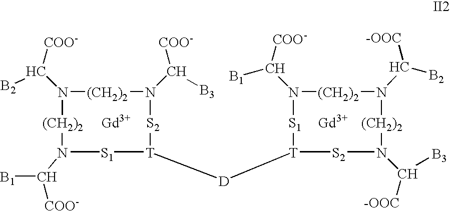
in which
   —S₁-T-S₂— is 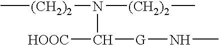
   where S₁=S₂=(CH₂)₂
all three of B₁, B₂ and B₃ representing (CH₂)ₓCONHR with x=1, 2 or 3 or A2 represents 2) 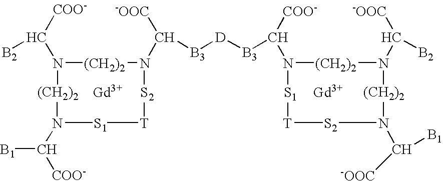
IIa2 (compound known as N-functionalized PCTA)
   Or IIb2 (compound referred to as N-functionalized PCTA and positional isomer of IIb2) of formula 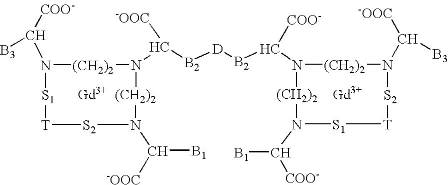
   in both of which S₁-T-S₂— is 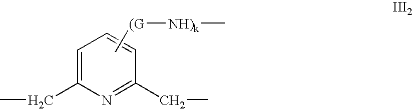
   with k=0 and S₁=S₂=CH₂;
   B₃ representing G-NH, and B1 and B2 representing (CH₂)ₓCONHR for IIa2 with x=1, 2 or 3,
   B₂ representing G-NH, and B1 and B3 representing (CH₂)ₓCONHR for II b2;
   or A2 represents 3) 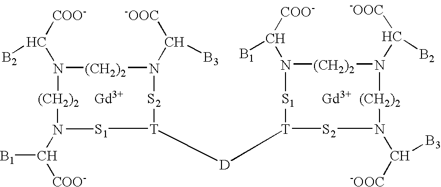
   IIc2 (compound referred to as C-functionalized PCTA)
   when S₁-T-S₂— is: 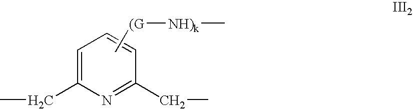
   with k=1 and S₁=S₂=CH₂;
all three of B₁, B₂ and B₃ represent (CH₂)ₓCONHR with x=1, 2 or 3 for II c 2, in the knowledge that, for II2, IIa2, IIb2 and IIc2,
GNH is chosen from the linkers 1 and preferably from the groups —(CH₂)ₙ—NH— or 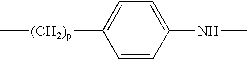
with p=to 3;
with n=1 to 4,
D being (Div−linker 2), with Div preferably being of 1,3,5-triazine type, D preferably being: 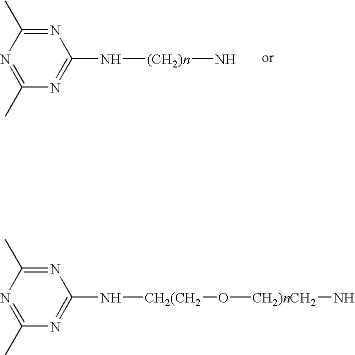
with n=2 to 6
R is as mentioned above.

In the compounds I1 and I2 above, the residues W1 and W2 derive from aliphatic polycarboxylic acids that are linear or cyclic or contain one or more phenyl rings. The latter are preferred in particular when the acids are attached to aromatic rings.

Among the aliphatic acids, mention may be made of (C₄-C₁₈)alkane-polycarboxylic acids in which the chain is optionally interrupted with a hetero atom as in C(CH₂O(CH₂)₂COOH)₄ described in OPPI Briefs 28(2), 1996, 242-244, or in ethylenediamine derivatives including diethylenetriaminopentaacetic acid, (C₅-C₆)cycloalkanepolycarboxylic acids, citric acid, 1,4,7,10-tetraazacyclododecane-1,4,7,10-tetraacetic acid or adamantane tetracarboxylic acid described in J. Org. Chem. 57(1), 1992, 358-362.

Among the aromatic acids, mention may be made of phenyl-, naphthyl- or biphenylpolycarboxylic acids, alkyl tri- or tetra(phenylcarboxylic) acids, phenyl- tri or tetra(phenylcarboxylic) acids such as 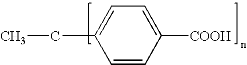
described in Chem. Ber. 123(2), 1990, 375-379,

or else derivatives of phosphazene such as the hexaacid: 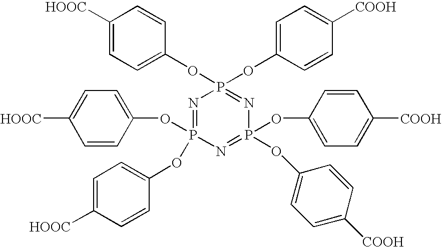
described in Macromolecules 22(1), 1989, 75-79 et 29, 1996, 3694-3700,

derivatives of phloroglucinol such as: 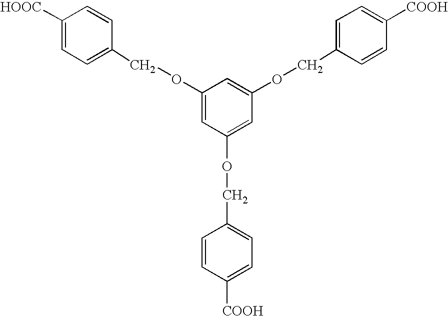
described in J. Org. Chem. 60(5), 1995, 1303-1308.

Finally, among the aliphatic acids, mention may be made of those containing aromatic rings, such as:

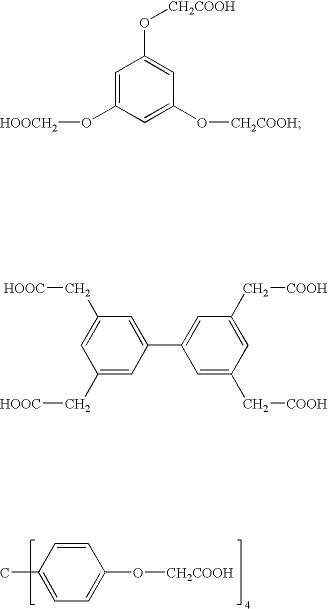

or the phenoxyacetic ether of hexahydroxyphosphazene.

Among the polyacids, those which will give biocompatible products will necessarily be chosen, given the application in diagnostics of the products of the invention. Preference is also given to the acids which give W1 and W2 residues which are rigid and/or in which the position of the carboxyl groups enables the A1 and A2 groups to be arranged uniformly in space around W1 and W2.

Finally, a criterion for selection may be the chemical accessibility to the starting polyacid and/or its reactivity with the derivatives of the chelates carrying an amine function.

The polyacids are known products or they may be prepared by conventional etherification, carboxylation and amidation processes, from known products.

Instead of a central nucleus W1 or W2 carrying carbonyl groups which form carboxamido groups with A1 or A2, use may be made, conversely, of a polyamino nucleus W1 or W2, the structure of the linkers 2 linking the central nucleus and the chelate(s) being adjusted accordingly so as to form amide, isothiocyanate or isocyanate bonds (known to those skilled in the art in Topics in Current Chemistry, New Class of MRI Central Agents, 221, Springer).

The Applicant has, moreover, studied compounds of the type polymetallic compounds of multimers: instead of having dividers D linked to two Gd chelates (r=2, therefore several dimers linked to the central nucleus), dividers D are linked to at least three Gd (several trimers linked to the central nucleus when r=3). Among the residues W1 or W2, preference is given to those derived from the acids: 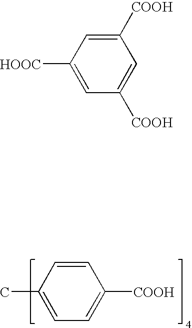
described in Angew. Chem. 98(12), 1986, 1095-1099; 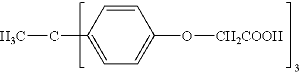
prepared according to U.S. Pat. No. 4,709,008; 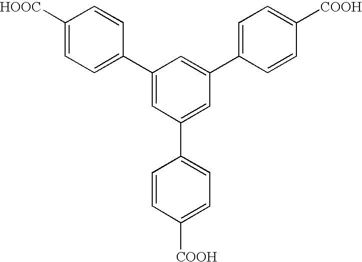
described in Chem. Ber., 123, 1990, 859-867; 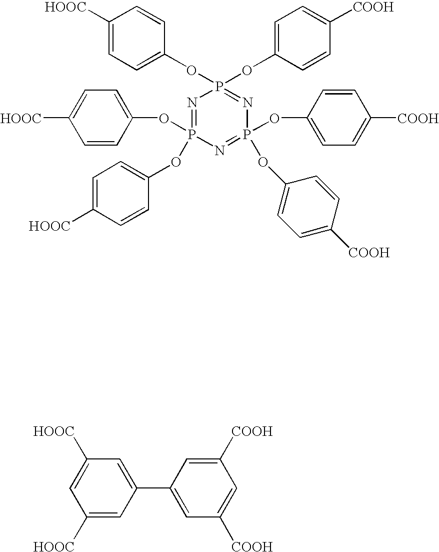
described in J. Org. Chem., 64 (7), 1999, 2422-2427,

and the residue of 1,3,5-triazine; in this case, the compound of formula I1 can be obtained by the action of the amine AH on commercial 2,4,6-trichlorotriazine.

Among the residues A1, mention may be made of those of the following three groups in which x and R have the meanings above:

those in which the macrocycle is cyclen, in. which, in formula I1,
—S₁-T-S₂—
represents: 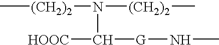
which have the formula: 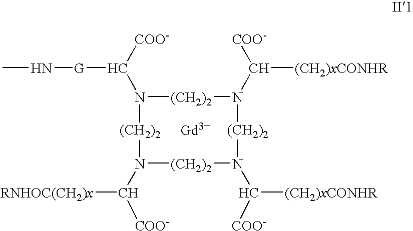
with -G-NH being —(CH₂)₃—NH— or

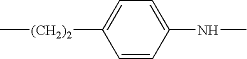

those in which the macrocycle is 3,6,9,15-tetraazabicyclo[9.3.1]pentadeca-1(15), 11,13-triene, functionalized on one of the aliphatic nitrogen atoms, of formula II1′ in which
—S₁-T-S₂— represents:

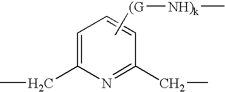

with k=0
of formula: 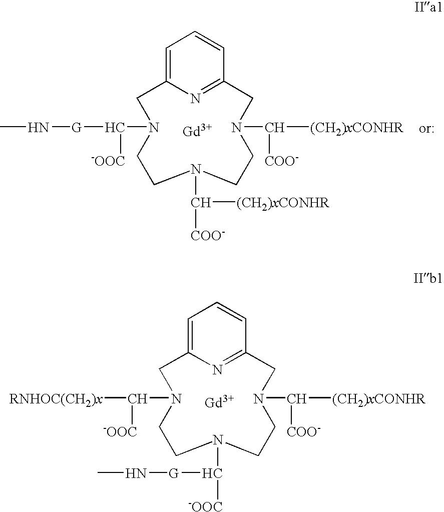
with -G-NH═—(CH₂)₃—NH— or

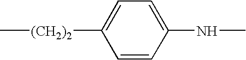

and those functionalized on the pyridyl ring, of formula II1 in which
—S₁-T-S₂—
represents: 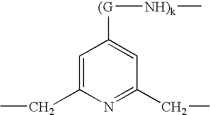
with k=1 and G=(CH₂)₃;

of formula:

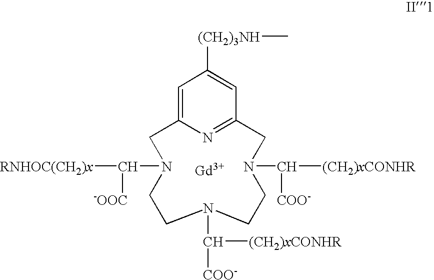

and especially the residues A1 in which x=2.

Similarly, among the compounds A2, mention may be made of compounds similar to the above three compounds II′1, II″a1, II″1, II′″1, replacing the Gd monomer with a Gd dimer, respectively II′2, II″a2, II″b2, II′″2.
1) 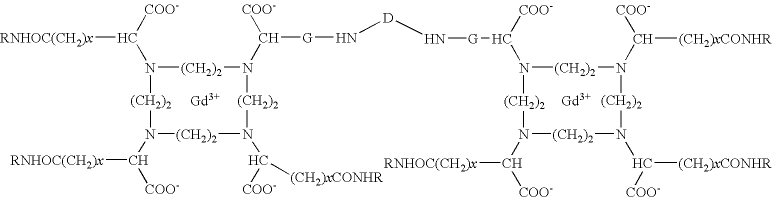
with -G-NH being —(CH₂)₃—NH— or 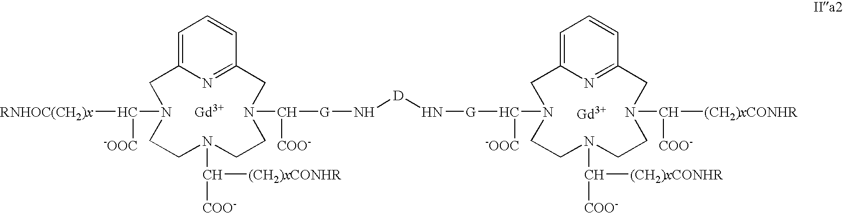
2)
Or II″b2 (positional isomer of II″a2) 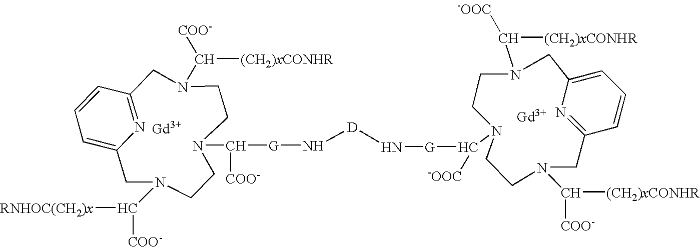
with -G-NH being —(CH₂)₃—NH— or
3) 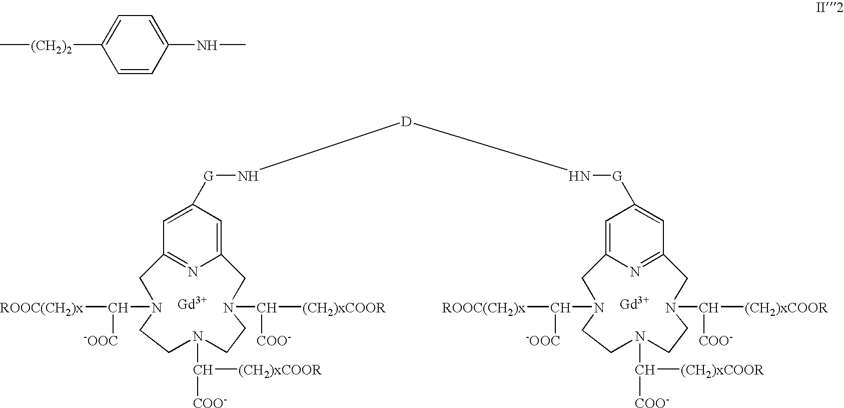
With G-NH being —(—CH₂)₃—NH—
and especially the residues A1 in which x=2.

The compounds of formulae II′₂, II″ₐ₂, and II″_{b2} are obtained starting from two equivalents of the compounds of structure V₁ as defined in the application, by means of a double substitution reaction on 2,4,6-trichloro-1,3,5-triazine in aqueous medium or in a mixture made up of water and of a water-miscible polar solvent, controlling the pH and the temperature.

The residues of formula R— are introduced by peptide coupling according to methods known to those skilled in the art, of the corresponding amines of formula R—NH₂ the structure of which was defined above, for example in aqueous medium in the presence of a compatible coupling agent such as EDCl and, optionally, a catalyst.

The third chlorine atom is finally displaced by means of a large excess of diamine, for example of formula H₂N—(CH₂)ₐ—NH₂ or H₂N—CH₂—(CH₂—O—CH₂)_{b}CH₂—NH₂ with a=2 to 5 and b=1 to 4.

The compounds of formula II′″₂ are prepared starting from the aminated precursors derived from the residues of formula II′″₁ and of structure: 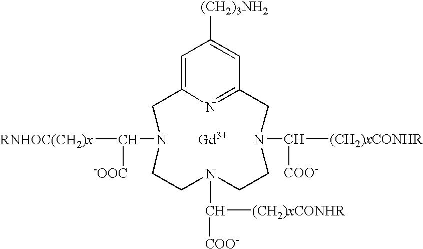
according to a similar protocol by means of double substitution of the triazine ring and displacement of the residual chlorine atom by means of a large excess of diamine as defined above.

Among the compounds of formulae I1 and I2, preference is given to those in which B₁, B₂ and B₃, when they do not represent G-NH, represent (CH₂)₂CONHR with, in R,
p=q=0 and Z=CH₂CONH, and in particular:
   either R represents R′: 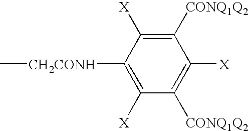
   or R represents R″: 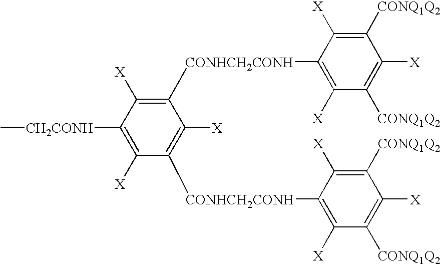
      and in R′ and R″ the X are identical and represent Br or I, while Q₁ and Q₂, which may be identical or different, are mono- or polyhydroxylated (C₁-C₈)alkyl groups, such that each CONQ₁Q₂ contains from 4 to 10 hydroxyls in total.

In addition, the compounds of formula I1 in which R comprises 2 or 3 phenyl rings have advantages for certain specific applications, due to their high molecular mass and/or volume. Among these, mention may be made more particularly of those in which R represents:

either R′″

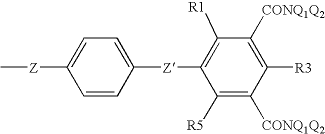

and Z is CH₂ or CH₂CONH and Z′ is CONH or CONHCH₂CONH;

or R″″:

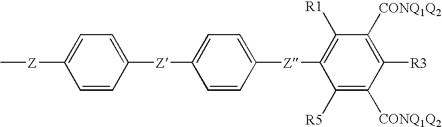

and Z is CH₂CONH, Z′ is CONH, and Z″ is CONHCH₂CONH;

and, in R′″ and R″″, R₁, R₃ and R₅, which are identical, are Br or I, and Q₁ and Q₂, which may be identical or different, are monohydroxylated or polyhydroxylated (C₁-C₈)alkyl groups, such that each group CONQ₁Q₂ contains from 4 to 10 hydroxyls in total.

Preference is also given to compounds (branches referred to as “flash” branches) in which R represents:

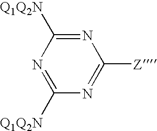

with Z″″ being NQCH₂(CH₂OCH₂)ᵢ(CH₂)ⱼNH₂, with i=2 to 6 and j=1 to 6

Finally, the compounds of formula I1 or I2 in which Q₁ represents CH₂CHOHCH₂OH or CH₂(CHOH)₄CH₂OH and Q₂ represents CH₂(CHOH)₄CH₂OH in particular in the groups CONQ₁Q₂ are generally preferred for forming molecules that are sufficiently hydrophilic in terms of aqueous solubility and of biocompatibility.

The products of formula I1 can be prepared either from the amines A1H, or from one of their precursors A′1NH, in particular the compounds in which there is (CH₂)ₓCOOH instead of (CH₂)ₓCONHR in B₁, B₂ or B₃. A1H and A′1NH, which are functionalized derivatives of macrocyclic gadolinium chelates, are synthesis intermediates of all the new compounds of formula I1, for which it was necessary to develop a preparation process suitable for the amino acid nature of the reactive group grafted onto the macrocycle for attaching the chelate onto the residue W1 without loss of stability of the macrocyclic chelate.

The invention therefore also relates to the precursors of the polymetallic compounds according to the invention, which are required for their synthesis.

According to one embodiment, the invention relates to the precursors A′1NH of formula: 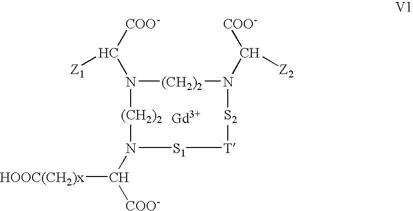
in which x=1, 2 or 3 and —S₁-T′-S₂— is:
1) 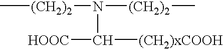
with S₁=S₂=(CH₂)₂
   or
2) 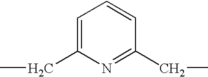
with S₁=S₂=CH₂
and one of the groups Z₁ or Z₂ is chosen from the groups —(—CH₂)₃NH₂ or 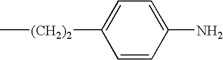
in which the NH₂ group may be optionally protected in a conventional manner, in the form of a carbamate, a phthalimide or a benzylamine as described, in general, in Protective Groups in Organic Synthesis, 3rd Ed., Ed. T. W. Greene, Pig. M. Wuts (J. Wiley) p. 494-653,
and the other of Z₁ or Z₂ is (CH₂)ₓCOOH.

The compounds V1 of 1) are referred to as being of RR DOTA type, and the compounds of 2) are referred to as being of N-functionalized RR PCTA type.

The invention also relates to the precursors A′1NH of formula: 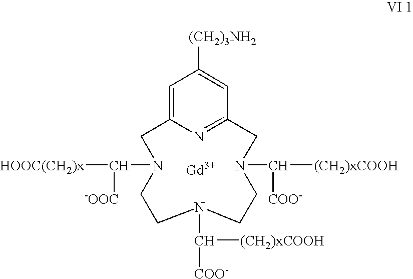
with x=1, 2 or 3
in which the NH₂ group is optionally protected or salified,
and particularly the compounds V1 and VI1 in which x=2,
and their salts with an alkaline base, such as NaOH or KOH.

These compounds VI1 are referred to as RR PCTA of the C-functionalized type, the amine function being located on the outer ring.

When W1 is the residue 1,3,5-triazino, the precursor V1, VI1 or VI′1 is preferably reacted on 2,4,6-trichloro-1,3,5-triazine under the usual conditions for a nucleophilic substitution in the presence of a base in an aprotic polar solvent, optionally mixed with water, in particular as described in Comprehensive Organic Chemistry, D. Bostow, W. Ollis, vol. 4, p. 150-152 (Pergamon Press) or in Tetrahedron Letters, 41(11), 2000, 1837-1840. The reaction may be carried out in the presence of an inorganic base such as NaOH or Na₂CO₃ or of a tertiary amine, such as triethylamine, for example in water in the presence of 5 to 60% by volume of 1,6-dioxane, of tetrahydrofuran or of dimethylformamide.

The chelates V1 or VI1, or optionally the product of condensation on W1, carry the acid groups: 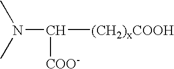
and can then be reacted with the amine RNH₂ in aqueous medium, optionally in the presence of a third solvent such as dioxane or tetrahydrofuran, with activation of the carboxylic groups by addition of a soluble carbodiimide, which carries an amine group, as described in J. Org. Chem., 21 (1956), 439-441 and 26 (1961), 2525-2528, or which carries a quaternary ammonium group such as 1-ethyl-3-(3-dimethylamino)carbodiimide (EDCl) or 1-cyclohexyl-3-(2-morpholino-ethyl)carbodiimide metho p-tolylsulfonate from Org. Synthesis vol. V, 555-558.

Among the other activators of the carboxylic acids in the amidation reactions, mention may also be made of N-hydroxysulfosuccinimide (NHS), Bioconjugate Chem. 5, 1994, 565-576, or 2-succinimido-1,1,3,3-tetramethyluronium tetrafluoroborate from Tetrahedron Letters, 30, 1989, 1927-1930. A mixture of EDCl and NHS can also be used.

A certain number of the amines RNH₂ required for preparing the compounds of formula I are known, described in particular in the abovementioned patent applications WO 97/01359, WO 00/75241 and EP-A-922700. The others may be prepared by means of similar processes, which come from the general knowledge of those skilled in the art.

When W1 is a polyacid derivative, it is preferred to prepare the amine A1H beforehand, from the chelates V1 or VI1 carrying the acid functions and optionally protected on the amine functions on which the appropriate amine RNH₂ is reacted, using one of the conventional methods of peptide amidation mentioned above.

To prepare the intermediate compounds of formula V1 in which Z₁ or Z₂ represents (CH₂)₃NH₂, it is possible to react, in a first step, on the corresponding macrocyclic compound in which the nitrogen atom carrying said group Z₁ or Z₂ is free and the other nitrogen atoms have been optionally protected, in a manner known per se, the compound Y′1-Br of formula: 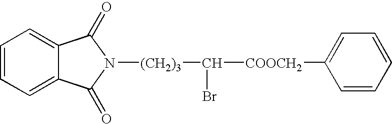
prepared according to Tetrahedron Letters 38(47), 1997, 8253-8256 and J. Org. Chem., 50, 1985, 560-565, whereas, for those in which Z₁ or Z₂ represents: 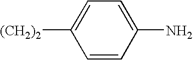
the compound Y″1-Br of formula: 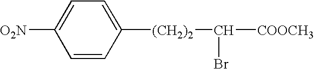
described in J. Org. Chem. 58, 1993, 3869-3876 is reacted.

The brominated diacid protected in ester form Y′″1 Br: 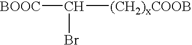
is then reacted on the other macrocyclic nitrogen atoms, after optional deprotection thereof, which brominated diacid can, for example, be prepared:
for x=1, B=diphenylmethyl,
   according to J.B.I.C. 4, 1999, 341-347;
   for x=2, B=(C₁-C₃)alkyl or benzyl,
   according to WO 00/75241;
   and for x=3, B=CH₃,
   according to EP-A-614 899,
      before freeing the amine function of the phthalimido group or reducing the nitro group, previously introduced. The acid functions are deprotected by the action of a base or of an acid in aqueous or aqueous-alcoholic medium, before or after the formation of the amino group.

After the carboxylic acid functions have been freed, the gadolinium complex is then prepared according to one of the methods known in particular from U.S. Pat. No. 5,554,748 or Helv. Chim. Acta, 69, 1986, 2067-2074, by the action of Gd₂O₃ on GdCl₃ in aqueous medium at pH of between 5 and 7.

During the preparation of the product of formula V in which T′ represents pyridyl, when, in the first step, Y′Br or Y″Br is reacted on the macrocycle in which none of the nitrogen atoms is blocked, the asymmetric derivatives of the type: 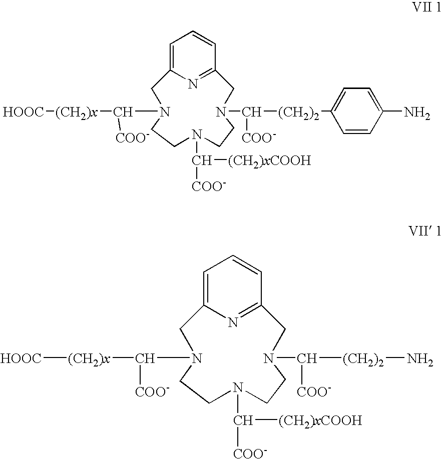
are obtained, after reaction with Y′″Br and formation of the amino group.

These compounds VII′1 are referred to as RR PCTA of the N-functionalized type, the amine function being located on a side arm.

To obtain the symmetrically substituted derivative, it is possible to use the process according to the reaction scheme of Table 1 below, in which x and B have the meanings above, starting with the protected triamine (a) described in Tetrahedron Letters, 41(39), 2000, 7443-7446, followed by steps similar to those mentioned for the asymmetric compound:

TABLE 1

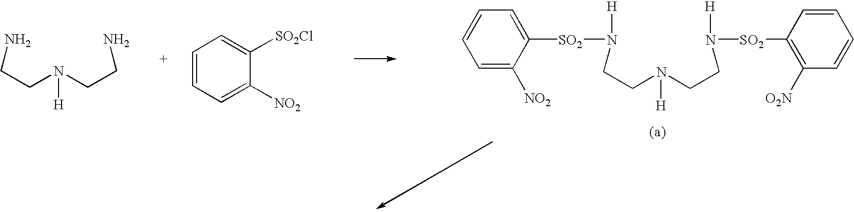

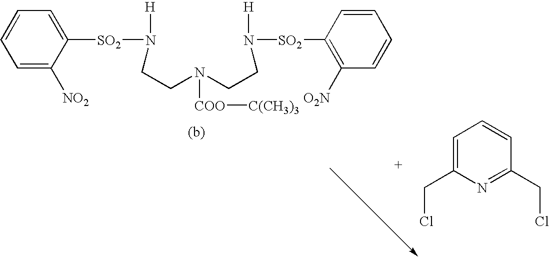

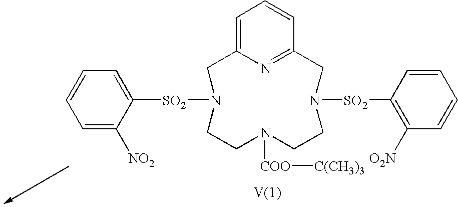

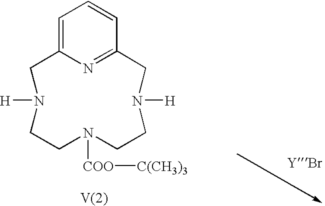

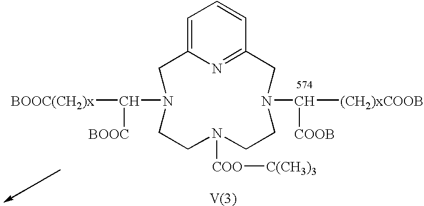

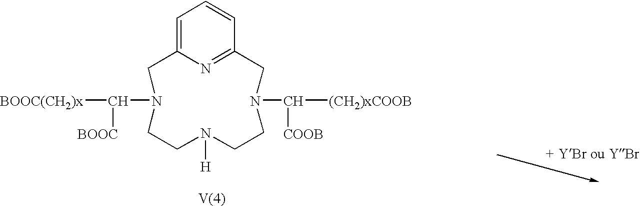

(The compounds V(1) to V(4) are intermediates of V1)

To prepare the compounds of formula VI1, a Heck reaction is carried out on the bicyclic macrocycle, brominated on the pyridyl ring of formula: 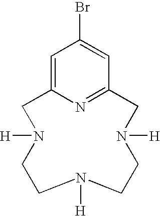
described in J. Heterocyclic Chem. 27, 1990, 167-169, followed by a reduction. The Heck reaction can be carried out under the conditions described in Metal Catalyzed cross-coupling reactions, Ed. F. Diederich, P. J. Stang, Wiley, VCH, chap. 3, p. 99-166. The reaction scheme for the first steps of the process for preparing VI is represented in Table 2; hydrolysis of the ester groups and the complexation of the gadolinium are then carried out, before or after deprotection of the amino group by the action of trifluoroacetic acid.

To prepare the amides resulting from the reaction of the compound VI with an amine RNH₂, it is preferred to carry out the amidation before deprotection of the aliphatic amine.

TABLE 2

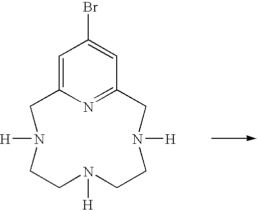

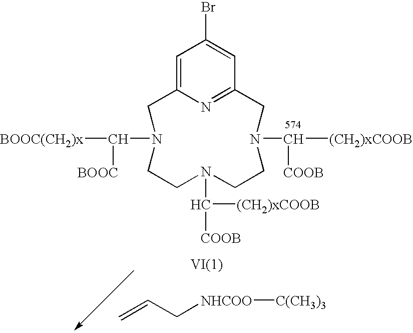

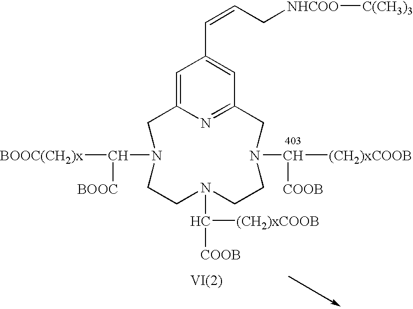

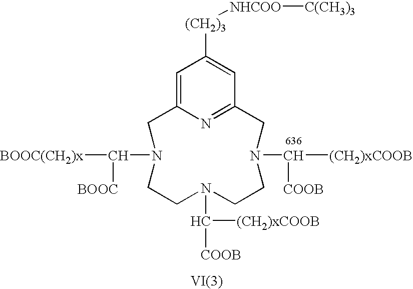

The compounds VI(1) to VI(4) are intermediates of VI1.

Similarly the invention also relates to the precursors A′₂NH that are dimers of formula:
1) 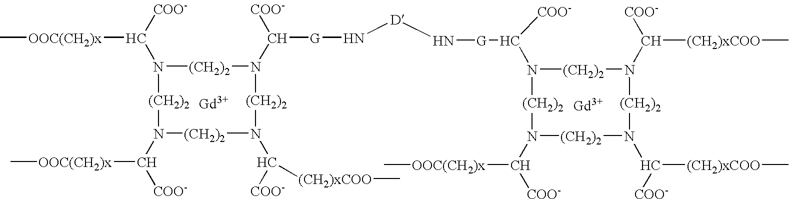
V2 precursor of II′2
in which x=1, 2 or 3, preferably x=2;
2) 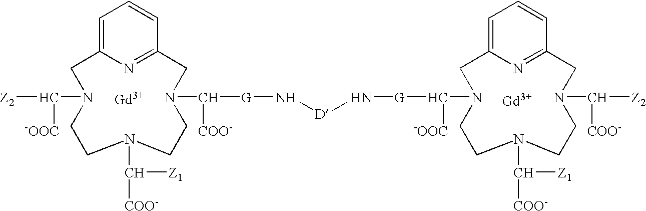
V″a2 precursor of II″a2
with, for 1) and 2):
   G-NH is chosen from the groups —(—CH₂)₃NH or 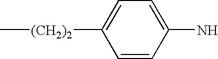
   with, for 2):
   Z₁ and Z₂ are (CH₂)ₓCOOH in which x=1, 2 or 3, preferably x=2;
3) 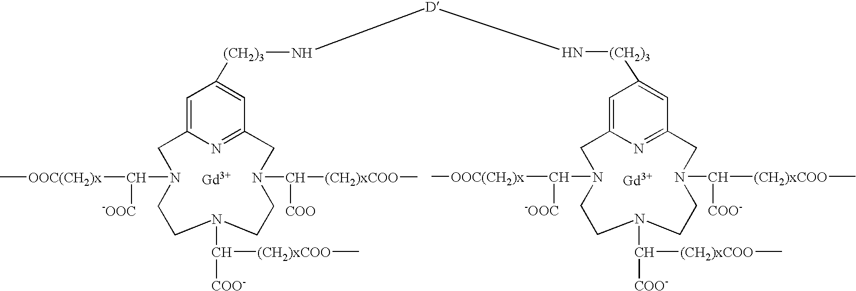
VI2 precursor of II′″2
with x=1, 2 or 3, preferably x=2.

The invention also relates to the contrast products for medical magnetic resonance imaging which comprise at least one compound of formula (E), preferably formula Poly M RR and Poly D RR, optionally combined with a carrier or with additives that are pharmaceutically acceptable for oral administration or administration by intravascular, subcutaneous or percutaneous injection. The diagnostic compositions for oral administration will be provided in the form of tablets or gel capsules, or oral suspensions and solutions. The aqueous solubility and the low osmolality of the compounds of the invention make it possible to prepare isotonic aqueous solutions of high concentration and of viscosity that is acceptable for injection.

According to another aspect, the invention relates to the paramagnetic complexes formed between the ligands of the invention and the suitable paramagnetic metal ions other than gadolinium, such as those of dysprosium or of manganese, and also the compositions of contrast agents for medical nuclear magnetic resonance imaging which comprise these complexes, combined with the usual carriers and additives. The ligands according to the invention can also form complexes with radioelements such as Tc, In or Yb, which can be used to establish a diagnosis or perform a therapeutic treatment. These complexes are in general provided in the form of an internal salt, resulting from the neutralization by the central metal cation of acid groups of the ligand; when the complex comprises other acid groups, they can be salified by means of a pharmaceutically acceptable inorganic or organic base, including amino acids, for example NaOH, lysine, N-methylglucamine, arginine, ornithine or diethanolamine. The doses at which the contrast agents according to the invention can be administered depend on the nature of the complex, on the relaxivity that it induces, on the route of administration and on the organ targeted. For example, when given orally, in particular for the gastrointestinal sphere, of the order of 0.01 to 3 mmol/kg of animal may be administered and, when given parenterally of the order of 0.001 to 0.02 mmol/kg may be administered.

Conventional additives can be introduced into the diagnostic compositions of the invention, such as buffers, antioxidants, electrolytes, surfactants, polyols, and other chelates of biological cations or of complexing agents in a small amount.

The solutions can be prepared extemporaneously from lyophilized powder containing the compound of formula (E) and, optionally, additives and a sterile solvent, or, due to the highly stable nature of the complexes in solution in vitro, just as in vivo, the solutions can be supplied to the radiologist in bottles or in syringes.

The unit doses will depend on the structure of the compound of formula E, on the route of administration, on the type of diagnosis to be established, and on the patient. The unit doses will in general be from 0.1 μmol to 150 μmol of gadolinium per kg, preferably from 1 to 100 μmol of gadolinium per kg, for a person of average size.

Due to their vascular confinement, their delayed elimination and their high relaxivity, the diagnostic compositions of the invention are useful for imaging blood vessels and lymphoid tissues. They make it possible to determine the perfusion and blood volume in pathological areas, to study microvascular permeability and to identify ischemic states or characterize tumors and inflammatory states.

The invention therefore relates in particular to the use of the diagnostic compounds of formula (E), and in particular of formula I1 or I2, for diagnosis by imaging, and to their use for preparing a composition for diagnozing these indications. The invention also relates to a medical imaging method of diagnosis using these compounds.

The invention also relates to a screening method, and. the compounds obtained by means of this screening method, consisting in selecting the compounds of formula (E) that are effective in diagnostic terms (pharmocokinetic and biodistribution properties), said method comprising:
preparing a candidate polymetallic compound of formula (E),
using the candidate compound in a test protocol suitable for a diagnostic indication,
selecting the candidates exhibiting a mass efficiency of at least 30%, preferably of at least 50%, greater than that of the corresponding non-polymetallic chelate.

As an example of a screening test, use will be made, for example of the in vitro and/or in vivo tests described in detail by the Applicant in the document Physical and biological evaluation of P792, a rapid clearance blood pool agent for Magnetic Resonance Imaging, Investigative radiology, vol 36, n^{o}8, 445-454, 2001.

Several examples of polymetallic compounds according to the invention are described by way of illustration.

Examples 1 to 13 and 39 concern polymetallics of monomers.

Examples 25 to 36, 38, 41 and 43 concern polymetallics of dimers; Examples 14 to 24, 37, 40 and 42 concern dimeric precursors of these polymetallics.

### EXAMPLE 1

Compound of formula VI1 with x=2 according to the method of Table 2

a) Compound of Formula VI(1) with B=ethyl

22 g of 13-bromo-3,6,9,15-tetraazabicyclo[9.3.1.]pentadeca-1(15), 11,13-triene are introduced into 440 ml of CH₃CN in the presence of 48 g of calcinated K₂CO₃ and the mixture is maintained at 80° C. for 1 h before adding a solution of 93 g of ethyl 2-bromoglutarate in 100 ml of CH₃CN the reaction medium is then stirred at 80° C. for 20 h and then cooled to ambient temperature and filtered, and the solvent is evaporated off.

The residue is taken up with 500 ml of an aqueous 1N HCl solution in the presence of one volume of diethyl ether. After separation of the organic phase, the aqueous phase is neutralized with NaHCO₃ and then extracted with CH₂Cl₂. After washing with water and then drying over magnesium sulfate, the organic phase is concentrated and the residue is purified on a column of silica (Merck® 500 g, d=10 cm), elution being carried out with CH₃COOC₂H₅.

m=37 g;

HPLC: column no. 1: Symmetry® C18; 100 Å; 5 μm; L=25 cm;

d=4.6 mm (Waters);

eluent no. 1: CF₃COOH in water (pH 3)/CH₃CN, linear gradient of 90/10 to 20/80 (v/v) in 40 min, flow rate 1 ml/min: tᵣ=26 min (2 peaks).

b) Compound of Formula VI(2)

23.5 g of 3-(tert-butyloxycarbonylamino)propene, 25.3 ml of triethylamine and then 3.4 g of triphenylphosphine and finally 1.8 g of palladium acetate are added to a solution of 28 g of the compound obtained in step a) dissolved in 400 ml of toluene.

After heating at 80° C. overnight under an inert atmosphere, the medium is evaporated off and the residue is taken up with an aqeuous hydrochloric acid solution (pH=1).

The aqueous phase is washed with 1 volume of diethyl ether and then of toluene before being brought to pH 6 by adding NaOH (1N).

After extraction of the aqueous solution with CH₂Cl₂, the organic phase, that has been dried over magnesium sulfate, is evaporated.

A brown oil is obtained.

m=17 g,

HPLC: column no. 1, eluent no. 1, linear gradient of: 60/40 to 20/80 (v/v) in 50 min, flow rate 1 ml/min: tr=14 min to 19 min (3 peaks of the isomers).

c) Compound of Formula VI(3)

3 g of catalyst palladium-on-charcoal at 10% are added to 17 g of the compound obtained in step b) dissolved in 350 ml of CH₃OH, and the reaction medium is then stirred at 20° C. for 2 h 30 min under 4×10⁵ Pa of hydrogen.

After filtration through Clarcel®, the solvent is evaporated off and 16.8 g of crude oil are obtained.

HPLC column no. 1, eluent no. 1, linear gradient of: 60/40 to 20/80 (v/v) in 50 min, flow rate 1 ml/min: tr=14-15-21 min (3 isomer peaks).

d) Hydrolysis of the Ethyl Ester Groups

20 g of the compound obtained in step c) dissolved in 50 ml of an aqueous 5N NaOH solution and 80 ml of CH₃OH are heated at 70° C. for 18 h.

After concentration of the reaction medium, the residue is taken up in water and the solution, brought to pH 5.5-6 with a few drops of acetic acid, is concentrated before being purified by chromatography on a column (d=15 cm) containing 1 kg of silanized silica (Merck® 0.063-0.200 μm), elution being carried out with water.

After concentrating to dryness, 9.3 g of white crystals are obtained.

HPLC column no. 1, eluent no. 2: H₂SO₄ in water (0.037 N)/CH₃CN linear gradient of: 98/2 to 20/80 (v/v) in 50 min:

tr=16.7-17.5-17.9 min (3 peaks).

e) Gadolinium Complexation

8.7 g of the compound obtained in step d) are dissolved in 70 ml of water and then 2.1 g of Gd₂O₃ are added in a single step, and the entire mixture is heated at 60° C. for 3 h 45 min, maintaining the pH between 5.5 and 6 by adding an aqueous 1N NaOH solution.

After filtration, the reaction medium is evaporated off and the residue is crystallized from ethanol.

9.6 g of white crystals are obtained.

HPLC: column no. 1, eluent no. 2, linear gradient of: 98/2 to 20/80 (v/v) in 50 min, flow rate 1 ml/min: tr=31 to 33 min (4 peaks).

f) Release of the Amine

A solution of 9 g of the complex obtained in step e) in 180 ml of CF₃COOH is maintained at 25° C. for 3 h with stirring, before eliminating the liquid under reduced pressure.

The residue is taken up in diethyl ether and the suspension is filtered. After elimination of the solvent, the residue is introduced portionwise into a suspension of at least 5 ml of weak anionic resin (OH⁻) in 50 ml of water when the addition is complete, the pH, which is stable, should be from 8 to 8.5.

The resin is then separated by filtration, the solvent is eliminated and the residue is precipitated by adding ethyl ether.

### EXAMPLE 2

Compound of formula V1 in which x=2, and

—S₁-T′-S₂— is, with S₁=S₂=CH₂,

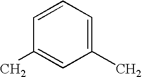

and Z₁ is

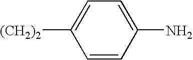

and Z₂ is (CH₂)₂—COOH prepared according to the method of Table 1.

a) N,N″-bis(o-nitrophenylsulfonyl)diethylenetriamine

8.4 g of NaOH crystals are dissolved in 100 ml of H₂O cooled to 0° C., and 9.2 g of diethylenetriamine are added, followed, dropwise at 0° C., by a solution of 41.5 g of 2-nitrophenylsulfonic acid chloride dissolved in 100 ml of tetrahydrofuran.

Once it has been concentrated, the reaction medium is taken up in CH₂Cl₂ and the organic phase is washed with H₂O and then dried over magnesium sulfate. After evaporation of the solvent, 37.5 g of crystals are obtained, which are used in the subsequent step.

HPLC: column no. 1, eluent no. 3: CF₃COOH in H₂O, pH 3.2: tr=16 min (32 min for the trisubstituted derivative).

b) N,N″-bis(2-nitrophenylsulfonyl)-N′-tert-butoxycarbonyldiethylenetriamine

18 g of di-tert-butyl carbonate are added fractionwise to a solution containing 32.4 g of compound obtained in step a) in a mixture of 97 ml of aqueous 2N NaOH solution and 225 ml of CH₃CN.

After stirring for 3 h at 25° C, the reaction medium is evaporated to dryness and the residue is taken up with 400 ml of CH₂Cl₂. The organic phase is washed twice with 100 ml of H₂O.

After drying over magnesium sulfate and then concentration of the organic phase, the residue obtained is purified by chromatography on a column (d=15 cm) containing 1 kg of silica (Merck® 40-63 μm), elution being carried out with a CH₂Cl₂/CH₃COCH₃ mixture, gradient from 99/1 to 90/10 (v/v).

After evaporation of the solvent, 28.5 g of product are obtained.

HPLC: column no. 1, eluent no. 1, flow rate 1 ml/min, linear gradient 90/10 to 10/90 (v/v) in 40 min: tr=29 min.

c) Compound of Formula V(1)

A solution containing 28 g of the compound obtained in step b) in 210 ml of CH₃CN in the presence of 41.4 g of calcinated K₂CO₃ is brought to reflux for 1 h 30 min.

After the addition of 11 g of 2,6-bis(chloromethyl)pyridine, the mixture is refluxed overnight.

The precipitate formed is filtered off, washed with 1 l of water, and then dried under vacuum m=26.8 g.

HPLC: column no. 1, eluent no. 1, flow rate 1 ml/min, linear gradient of: 90/10 to 10/90 in 40 min then 0/100 (v/v) in 10 min: tr=32 min.

d) Compound of Formula V(2)

16 g of LiOH are added to 20 g of the compound obtained in step c) in suspension in 310 ml of dimethylformamide, followed, dropwise, by 18 g of thioglycolic acid, in ½ hour.

The red-colored reaction medium is stirred for 6 h at 25° C. and then 400 ml of H₂O and 400 ml of CH₂Cl₂ are added thereto. After stirring and settling out, the aqueous phase is separated and extracted with 400 ml of CH₂Cl₂. This organic phase, after having been washed twice with water, is combined with the preceding phase and the entire mixture is concentrated. The oily residue is purified by flash chromatography on silica (Merck®, 40-63 μm), elution being carried out with a CH₃OH/NH₄OH mixture (50/1) after elimination of the impurities by elution with CH₃OH.

After evaporation of the fractions that are in conformity, 5.5 g of product are obtained.

HPLC: column no. 1, eluent no. 4: PIC B8 (Waters)/CH₃CN, flow rate 1 ml/min, linear gradient of: 90/10 to 10/90 (v/v) in 40 min: tr=14 min (tr=32 min for the starting product).

e) Compound of Formula V(3) with x=2, B=Ethyl

22 g of ethyl 2-bromoglutarate and 11 g of calcinated K₂CO₃ are added to a solution of 8 g of compound obtained in step d) in 60 ml of CH₃CN and 26 ml of diisopropyl ether, and the mixture is then brought to its reflux temperature for 24 h.

After filtration and then evaporation under vacuum, the oil obtained is purified by flash chromatography on silica (Merck®, 40-63 μm), elution being carried out with a heptane/ethyl acetate mixture (60/40 v/v).

After evaporation, 7.7 g of oil are obtained.

HPLC: column no. 1, eluent no. 1, flow rate 1 ml/min, linear gradient of: 98/2 to 10/90 (v/v) in 50 min: tr=31 and 32 min (2 peaks) (starting product tr=4 min).

f) Compound of Formula V(4)

5.3 g of the compound obtained in step e) are dissolved in 32 ml of trifluoroacetic acid and the mixture is stirred for 1 h 30 min at 25° C.

After concentrating the reaction medium under vacuum, the oil obtained is purified by flash chromatography on silica (Merck®, 40-60 μm), elution being carried out with a CH₂Cl₂/CH₃OH mixture (97/3, v/v).

After elimination of the solvent, 3.8 g of solid product are obtained.

HPLC: column no. 1, eluent no. 1, flow rate 1 ml/min, linear gradient of: 98/2 to 10/90 (v/v) in 50 min: tr=23 min.

¹³C NMR (125 MHz−DMSOd6−30° C.) δ (ppm) 160.4 (C_{1,11}); 119.8 (C_{12,14}); 138 (C₁₃); 52.9 (C_{2,10}); 51.6 (C_{4,8}); 45.0-45.3 (C_{5,7}); 172.2 (C═O); 14.3 (CH₃—CH₂); 59.7-60.3 (CH₃—CH₂—O); 65.4 (C—N); 25.4-25.5; 30.4-30.5 (C—CH₂—CH₂—CO).

g) Reaction with Y″Br=

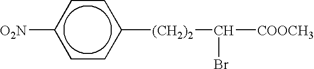

17 g of the compound Y″Br are added to a suspension of 9.4 g of the compound obtained in step f) in 30 ml of diisopropyl ether and 65 ml of CH₃CN in the presence of 4.5 g of calcinated K₂CO₃.

After stirring for 48 h at 85° C., the reaction medium is filtered and concentrated under vacuum, and the residual oil is purified by chromatography on a column (d=15 cm) containing 1 kg of silica (Merck® 40-60 μm), elution being carried out with a 70/30 v/v CH₂Cl₂/acetone mixture.

After concentrating to dryness, 5 g of product are obtained.

HPLC: column no. 1, eluent no. 1, flow rate 1 ml/min, linear gradient of: 98/20 to 10/90 (v/v) in 50 min: tr=26 min.

h) Hydrolysis of the Ethyl Ester Groups

4 g of the compound obtained in step g) are added to a solution of 10 ml of 12N HCl, and the mixture is then stirred for 48 h at its reflux temperature.

After filtration and concentration, the residue is purified by chromatography on silanized silica gel (Merck® 0.063-0.20 μm), elution being carried out with an H₂O/CH₃OH mixture, to give 2.2 g of product.

HPLC: column no. 1, eluent no. 1, flow rate 1 ml/min, linear gradient of: 98/2 to 10/90 (v/v) in 50 min: tr=16 min (2 peaks).

i) Gadolinium Complexation

2.2 g of the compound obtained in step h) are introduced into 40 ml of H₂O.

The pH of the suspension is brought to 5 by adding an aqueous 2N NaOH solution and the medium is then heated to 50° C. until complete solubilization is obtained.

After the addition of 0.6 g of Gd₂O₃, the solution, always maintained at pH 5 by adding an aqueous 2N NaOH solution, is heated at 80° C. for 6 h.

After having filtered off the salts by evaporation, the residue is crystallized from C₂H₅OH m=2 g.

HPLC: column no. 1, eluent no. 1, flow rate 1 ml/min, linear gradient of: 96/2 to 10/90 (v/v) in 50 min: tr=17 and 21 min (2 peaks).

j) Reduction of the Nitro Group

0.4 g of catalyst palladium-on-charcoal at 10% is added to 2 g of the compound obtained in step i) dissolved in 50 ml of H₂O, and the reaction medium is then stirred at 25° C. under a hydrogen pressure of 3×10⁵ Pa for 6 h. After elimination of the catalyst by filtration through a Millipore® filter (0.45 μm and 0.22 μm), the solution is evaporated to give 1.8 g of product.

HPLC: column no. 1, eluent no. 1, flow rate 1 ml/min linear gradient of: 98/2 to 10/90 (v/v) in 50 min: tr=10 min.

### EXAMPLE 3

Compound of Formula VII1 in which x=2

a) A solution of 102 g of the ester methyl 2-bromo-4-nitrophenylbutyrate in 100 ml of CH₃CN is added to a suspension, of 70 g of 3,6,9,15-tetraazabicyclo[9.3.1.]pentadeca-1(15),11,13-triene in 800 ml of CH₃CN in the presence of 910 ml of anion exchange resin in the form of a strong base (Amberlite® IRA458).

After stirring at 25° C. for 3 days, filtration of the resin and evaporation, the oil obtained is purified by chromatography on a column of 5 kg of silica (Merck®, 40-60 μm), elution being carried out with a CH₂Cl₂/CH₃OH mixture (70/30 v/v). 38 g of product are obtained.

HPLC: column no. 1, eluent no. 1, but pH 3, flow rate 1 ml/min, linear gradient of: 98/2 to 10/90 in 50 min: tr=15 min.

¹³C NMR (125 MHz, DMSOd6, 30° C.) δ (ppm): 160.1 (C₁); 53.8 (C_{2,4}); 45-45.4-45.7 (C_{5,7,8}); 51.3 (C₁₀); 161.6 (C₁₁); 119.3 (C₁₂); 119.6 (C₁₄); 137.6 (C₁₃) 51.7 (O—CH₃); 172.8 (C═O); 65.8 (C—N); 31.06-31.45 (CH₂—CH₂); 149.6-129.6-122 (Ar); 145.6 (Ar—NO₂).

b) Reaction with Y′″Br=

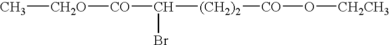

6.8 g of K₂CO₃ and 13 g of ethyl 2-bromoglutarate are added to a solution of 7 g of the compound obtained in step a) in 70 ml of CH₃CN and 35 ml of diisopropyl ether, and the mixture is left at reflux for 24 h with stirring.

After elimination of the salts by filtration and concentration of the solution, the oil obtained is purified by chromatography on silica (Merck® 40-63 μm), elution being carried out with a CH₂Cl₂/acetone mixture (70/30 v/v).

6 g of solid product are obtained.

HPLC: column no. 1, eluent no. 1, flow rate 1 ml/min, linear gradient of: 98/2 to 10/90 (v/v) in 50 min: tr=33 min.

c) Hydrolysis of the Ethyl Ester Groups

2.8 g are obtained from 6 g of the compound obtained in step b), by applying the same procedure as for step h) of Example 2.

HPLC: column no. 1, eluent no. 1, gradient of: 98/2 to 10/90 (v/v) in 50 min; flow rate 1 ml/min: tr=17 to 19 min (3 peaks).

d) Gadolinium Chelate of the Preceding Compound

2 g of GdCl₃, 6H₂O are introduced into 35 ml of a solution, at pH 5, of 3.9 g of the compound obtained according to step c), and the mixture is maintained at 50° C. for 5 h, during which time the pH is adjusted if necessary by adding an aqueous NaOH solution (2N).

Next, the medium is filtered and then evaporated; 4 g of weakly acidic cation exchange resin Chelex® 100 (Bio-Rad) are added to the oil obtained, dissolved in 40 ml of water.

After stirring at 25° C. for 2 h, the resin is eliminated by filtration and the solution is evaporated to give 4.5 g of product.

HPLC: column no. 1, eluent no. 1, gradient of: 98/2 to 10/90 (v/v) in 50 min, flow rate 1 ml/min: tr=15.6 to 18.7 min (several peaks).

e) Reduction of the Nitro Group

4 g of product are obtained from 4.5 g of the compound obtained in step d), by applying the same procedure as in step j) of Example 2.

HPLC: column no. 1, eluent no. 1, linear gradient of: 98/2 to 10/90 (v/v) in 50 min, flow rate 1 ml/min: tr=8.6 to 9.5 min (several peaks).

### EXAMPLE 4

Compounds of Formula V1 where x=2,

and —S₁-T′-S₂— is, with S₁=S₂=(CH₂)₂,

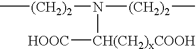

while Z₁ is

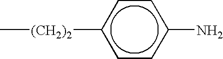

and Z₂ is —(—CH₂)₂—COOH

a) 40.4 g of methyl 2-bromo-4-(4-nitrophenyl)butyrate in solution in 50 ml of CH₃CN are added dropwise to a suspension of 20 g of 1,4,7,10-tetraazacyclododecane in 140 ml of CH₃CN.

After stirring at 25° C. for 24 h, the solution is filtered, and washed with CH₃CN then with 200 ml of diethyl ether.

After filtration, the product in hydrobromide form is recrystallized from 200 ml of CH₃CN.

m=42 g; mp=170° C.

HPLC: column no. 2 Lichrospher® Merck® C₁₈, 100 Å, 5 μm, L=25 cm, d=4.6 mm, eluent no. 5: KH₂PO₄ in H₂O (0.01 M)/CH₃CN (45/55 v/v), flow rate 1 ml/min: tr=2.5 min.

b) Reaction with Y′″Br=

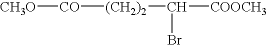

A suspension containing 20 g of the compound obtained in step a) and 20 g of Na₂CO₃ in 400 ml of CH₃CN is brought to reflux temperature for 15 min before adding 40 g of methyl 2-bromoglutarate, dropwise.

After stirring at reflux for 24 h and then at 25° C. overnight, the medium is filtered and the solvent is then evaporated off and the residue is dissolved in 100 ml of CH₂Cl₂. The organic phase is washed with water and then dried over sodium sulfate, before elimination of the solvent by evaporation under reduced pressure. The residue is dissolved in a minimal volume of aqueous 1 M HCl solution. This solution is washed with the same volume of diethyl ether and then brought to pH 4 with NaHCO₃, before being extracted with diethyl ether. After evaporation of the organic phase, the residue is purified by chromatography on silica (Merck® Si 60), elution being carried out with a heptane/CH₃COOC₂H₅ mixture (40/60 v/v then 30/70 v/v); m=8 g.

HPLC: column no. 1 eluent no. 1 (pH=2.8), flow rate 1 ml/min, linear gradient of: 90/10 to 60/40 (v/v) in 20 min then 20/80 in 10 min: tr=22 to 29 min (3 isomer peaks).

c) Hydrolysis of the Methyl Ester Groups

10 g of the compound obtained in step b) are dissolved in 20 ml of an aqueous 12N HCl solution and the mixture is brought to reflux for 24 h. After cooling, the solution is evaporated and the residue is dissolved in water. After concentrating under vacuum, 7.7 g of crude product are obtained.

d) Complexation of the Gadolinium with the Preceding Compound

The solution of 5 g of crude product above in 30 ml of H₂O is brought to pH 5.2 by adding 5M NaOH, before adding 1.2 g of Gd₂O₃.

The medium is heated at 80° C. for 2 h 30 min, during which time the pH is maintained between 5.2 and 5.5 by adding an aqueous 6M HCl solution.

After cooling to 25° C., the medium is poured into 250 ml of C₂H₅OH at 10° C. The precipitate obtained after washing with C₂H₅OH is dried; m=5 g.

HPLC: column no. 1, eluent no. 1 (pH=2.8), flow rate=1 ml/min, linear gradient of 90/10 to 85/15 (v/v) in 15 min, then 70/30 in 15 min, then 40/60 in 10 min: tr=31 to 34 min (3 peaks).

e) Reduction of the Nitro Group

5 g of the gadolinium complex in the form of the sodium salt are dissolved in 70 ml of water and hydrogenated under pressure as in step j) of Example 2. 5 g of product are obtained in the form of the sodium salt.

HPLC: column no: 1, eluent no. 1 (pH=2.8), flow rate 1 ml/min, gradient of: 98/2 to 85/15 (v/v) in 20 min, then 70/30 in 20 min: tr=17 to 21 min (3 peaks).

### EXAMPLE 5

Compounds of Formula V1 where x=2, —S₁-T′-S₂— is, with S₁32 S₂=(CH₂)₂,

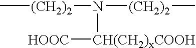

Z₁ is —(—CH₂)₃—NH₂

Z₂ is —(—CH₂)₂COOH

a) 49 g of benzyl 2-bromo-5-phthalimidopentanoate (compound Y′Br) in solution in 235 ml of CH₃CN are added dropwise, in 30 min, to a stirred suspension of 20 g of 1,4,7,10-tetraazacyclododecane in 235 ml of CH₃CN.

After leaving the mixture at 25° C. overnight, the precipitate obtained is filtered and then washed with a minimum amount of CH₃CO₂C₂H₅.

m=45 g (amine and hydrobromide mixture).

HPLC: column no. 1, eluent no. 1 (pH=2.8) for 5 min, then linear gradient of: 97/3 to 60/40 v/v in 35 min; flow rate 1 ml/min: tr=26 min.

b) Reaction with Y′″Br=

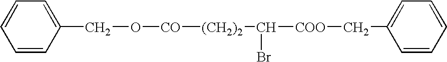

A solution consisting of 109 g of benzyl 2-bromoglutarate in 200 ml of dry CH₃CN is added rapidly to a suspension of 45 g of the compound obtained in step a) in 500 ml of CH₃CN, preheated to 80° C.

35 g of Na₂CO₃ are added at the end of the addition, and the medium is then brought to reflux for 48 h.

After cooling, the reaction medium is filtered, the solvent is evaporated off under reduced pressure and the oil obtained is purified by chromatography on silica, elution being carried out with pure CH₂Cl₂ and then with a CH₂Cl₂/CH₃CO₂C₂H₅ mixture: (95/5 v/v); m=41 g.

c) Hydrolysis of the Benzyl Ester Groups and Release of the Amine

A solution of 5 g of the crude product obtained in step b) in 25 ml of aqueous 2N HCl solution is brought to reflux for 24 h. After cooling, 100 ml of water are added, before extracting the aqueous phase with three times 50 ml of diethyl ether.

After evaporation of the aqueous phase, 2.5 g of product are obtained, which are purified by filtration of the aqueous solution on silanized silica (Merck® Si60); m=2.2 g.

HPLC: column no. 3: Hyper Carb® (hypersil) 5 μm 250 Å; L=25 cm; d=4.6 mm (Waters); eluent no. 6: H₂SO₄ in H₂O (0.1% m/v)/CH₃CN, linear gradient of: 98/2 to 85/15 (v/v),in 20 min then 50/50 in 30 min: tr=21 to 28 min (several peaks).

d) Gadolinium Complexation

1.4 g of expected product are obtained starting from 2.2 g of the compound obtained in step c) above, by applying the same procedure as for step d) in Example 4.

HPLC: column no. 1, eluent no. 6, 100/0 (v/v) for 5 min, then gradient of 100/0 to 85/5 (v/v) in 25 min then 50/50 in 20 min flow rate 1 ml/min: tr=22 to 26 min (several peaks).

### EXAMPLE 6

Compound of Formula A1H in which A1 is the Formula II′″1, with x=2

and R=

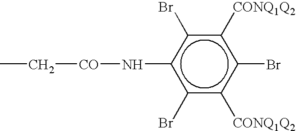

with Q₁=CH₂CHOHCH₂OH and Q₂=CH₂(CHOH)₄CH₂OH

a) 16 g of the compound obtained in step e) of Example 1 and 61 g of the amine RNH₂ are dissolved in 600 ml of H₂O. The solution is brought to pH 6 by adding an aqueous 1N NaOH solution and then 20 g of 1-(3-dimethylaminopropyl)-3-ethylcarbodiimide hydrochloride (EDCl) and 1.1 g of (N-hydroxysuccinimidyl)-3-sulfonic acid (NHS) are added.

After 18 h at 25° C., during which time the pH is maintained at 6 by adding an aqueous solution of 1N NaOH or of 1N HCl, the solvent is evaporated off and the residue is then precipitated from C₂H₅OH.

The product obtained by filtration is then purified by chromatography on a column of silanized silica, elution being carried out with H₂O and then with H₂O/CH₃OH mixtures.

After evaporation of the fractions which are in conformity, the residue dissolved in water is treated with 30 ml of anionic resin (OH³¹). The solution, filtered once and then evaporated, gives 22 g of white crystals.

HPLC: column no. 1, eluent no. 3, linear gradient of: 98/2 to 60/40 (v/v) in 50 min; tr=18 min.

b) Deprotection of the Amino Group

21 g of the preceding compound dissolved in 400 ml of CF₃COOH are stirred at 25° C. for 3 h.

After evaporation of the solvent, the residue is crystallized from diethyl ether. After filtration, the product obtained is added portionwise to 10 ml of OH⁻ anionic resin (Amberlite® IRA67) in 100 ml of water, always maintaining the pH of the solution above 5 during the addition by possibly adding resin. Once the addition is complete, the solution, the pH of which is stabilized at around 8, is filtered so as to eliminate the resin and then evaporated. After drying, 20 g of white crystals are obtained.

HPLC: column no. 1, eluent no. 3, linear gradient of: 98/2 to 60/40 (v/v) in 50 min: tr=7 min (unresolved peak) (tr=19 min for the starting product).

### EXAMPLE 7

Compound of Formula I1 where W1 is

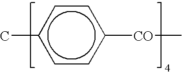

m=4,

A1 represents the group of formula II′″1 where x=2 and R=

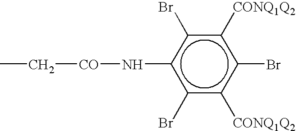

with Q₁=CH₂CHOHCH₂OH and Q₂=CH₂(CHOH)₄CH₂OH

1.6 g of EDCl, 1.2 g of 1-hydroxybenzotriazole (HOBT) and 1.7 ml of triethylamine are added to a solution of 30 ml of dimethyl sulfoxide containing 0.5 g of tetrakis(4-carboxyphenyl)methane acid and 20 g of the compound obtained according to step b) of Example 6.

After stirring at 25° C. for 36 h, the solution is poured into 100 ml of C₂H₅OH and the precipitate obtained is separated by filtration and then redissolved in 40 ml of water. The solution is ultrafiltered through a polyethersulfone membrane (Pall®) with a cut-off threshold of 5 KD and then through a membrane with a cut-off threshold of 10 KD. After evaporating off the water, 10 g of crystals are obtained.

HPLC: column no. 1, eluent no. 3, linear gradient of: 98/2 to 50/50 (v/v) in 50 min: tr=13 min.

SEC (steric exclusion chromatography):

conditions no. 1: carried out on a succession of 4 columns (d=8 mm, I=30 cm) sold by Shodex® (JP) under the references OH Pack SB-HQ, containing polyhydroxymethacrylate gel, the exclusion limits of which, determined with Pullulan®, are successively: 10⁶ KD (SB-804); 10⁵ KD (SB-803); 10⁴ KD (SB-802.5); 10⁴ KD (SB 802.5); eluent: aqueous NaCl solution (0.16M)/CH₃CN 70/30 v/v, flow rate 0.8 ml/min. T=30° C.: tr=36 min.

### EXAMPLE 8

Compound of Formula I1 with W1 is

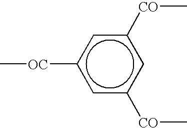

m=3

A1 represents the same group as that of Example 7.

26 g of white solid are obtained from 0.6 g of 1,3,5-benzenetricarboxylic acid, after elimination of the excess RNH₂, by applying the same procedure as for Example 7.

HPLC: column no. 1, eluent no. 3; linear gradient of: 98/2 to 50/50 (v/v) in 50 min: tr=12 min.

SEC: conditions no. 1 tr=37 min.

### EXAMPLE 9

Compound of Formula I1 with W1 is

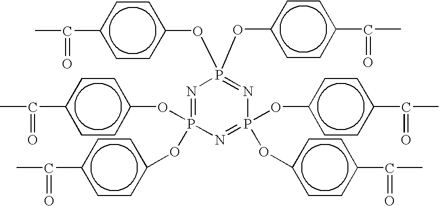

m=6

and A1 represents the same group as in Example 7.

By applying the same procedure as for Example 7, 8 g of crystals are obtained, after elimination of the excess of RNH₂, from 0.5 g of the hexaacid derived from cyclotriphosphazene of formula:

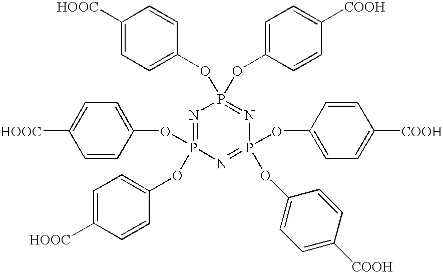

HPLC: column no. 1, eluent no. 3, linear gradient of: 98/2 to 50/50 (v/v) in 50 min: tr=14 min.

SEC: conditions no. 1: tr=35 min.

### EXAMPLE 10

Compounds According to Formula I1 with W1

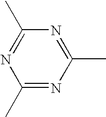

m=3

and A1 represents the group of formula II′1 where x=2, -GNH— is

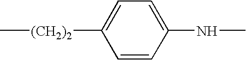

and R is

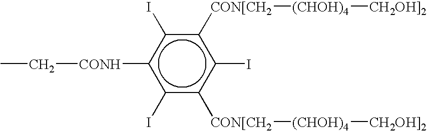

a) Condensation with the Central Nucleus

A solution of 0.2 g of 2,4,6-trichloro-1,3,5-triazine in dioxane is added, in 10 min, to a solution containing 4 g of the compound obtained in step e) of Example 4 in 30 ml of distilled water, heated to 60° C., and the pH is then brought to 8.4 by adding an aqueous 1N NaOH solution.

The reaction medium is stirred for 4 days, during which time the pH is regularly brought to 8.4 by adding an aqueous 0.2M NaOH solution.

After the addition of a volume of water to the reaction medium and then ultrafiltration through a polyethersulfone membrane (Pall®) with a cut-off threshold of 1 KD, the retentate (40 ml) is concentrated to a volume of 10 ml and then poured into an aqueous 1N HCl solution. The precipitate formed is isolated. m=2.5 g.

SEC: conditions no. 1: tr=40 min.

b) Amidation Reaction

2.3 g of EDCl and 0.2 g of NHS are introduced into a solution, at pH 6, containing 2.5 g of the compound obtained in step a) above and 11.5 g of the iodinated amine of formula RNH₂ in 40 ml of H₂O; the mixture is stirred at 25° C. for 3 h and the solution is then poured into 200 ml of cold C₂H₅OH.

The precipitate obtained is redissolved in 100 ml of water in order to be purified by ultrafiltration through a polyether sulfone membrane (Pall®) with a cut-off threshold of 10 KD.

9 g of white solid are isolated by elimination of the solvent from the retentate.

SEC: conditions no. 1: tr=37 min (44 min for RNH₂).

### EXAMPLE 11

Compound According to Formula I1 where W1 is

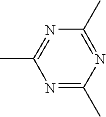

m=3

and A1 represents the group of formula II′ where x=2, and GNH— is

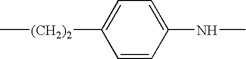

and R is

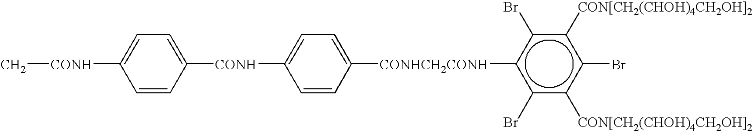

By applying the same procedure as for Example 10, step b), 10 g of white product are obtained starting from 2.5 g of the compound of Example 10a and 14 g of amine RNH₂, and then eliminating the molecular weight impurities.

SEC: conditions no. 1: tr=36 min (tr=45 min for RNH₂)

### EXAMPLE 12

Compound According to Formula I1 where W1 is

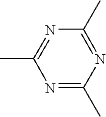

m=3

and A1 represents the group of formula II′ where x=2, -GNH— is

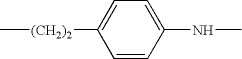

and R is 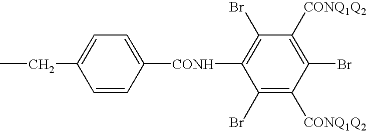
with Q₁=CH₂CHOHCH₂OH
   Q₂=CH₂(CHOH)₄CH₂OH

SEC: conditions no. 1; tr=37 min (tr=45 min for RNH₂).

### EXAMPLE 13

Compound According to Formula I1 where W1 is:

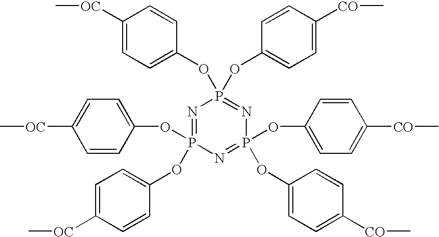

m=6

and A1 represents the formula II′″1 where x=2

R is

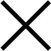

with Q₁=Q₂=CH₂(CHOH)₄CH₂OH and X=Br.

a) Preparation of the Intermediate Chelate of Formula VIII

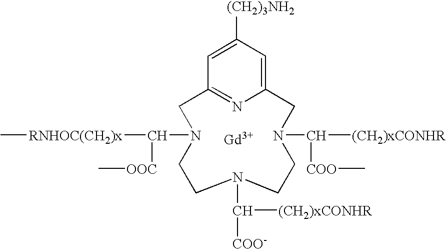

and R represents

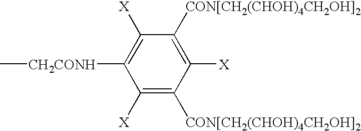

1-6 g of compound obtained in step e) of Example 1 and 26.5 g of amine RNH₂ are dissolved in 200 ml of water and 7.6 g of EDCl and 0.4 g of NHS are added. The mixture is maintained at around pH 6 for 24 hours, with stirring, with addition of an aqueous N NaOH or HCl solution if necessary.

After evaporation of the solvent, the residue is crystallized by adding ethanol. The 35 g of yellow crystals obtained are dissolved in 200 ml of water and the solution is ultrafiltered with a polyethersulfone membrane (Pall®) with a cut-off threshold of 1 kD.

The retentate is concentrated and purified by chromatography on a column of silanized silica (Merck®) (diameter: 7 cm, height: 33 cm), elution being carried out with water and then water/methanol mixtures (90/10 VN to 80/20). The fractions containing the desired product are concentrated until the solvents are eliminated.

The residue, dissolved in 50 ml of water, is treated with 20 ml of anionic resin in OH⁻ form (HP 661 from Rohm and Haas) and then treated with carbon black at 45° C.

After filtration and elimination of the solvents, 10 g of white crystals are isolated.
HPLC: column no. 1, eluent no. 7: H₂O/CH₃CN, linear gradient 98/2 to 60/40 (V/V) in 50 min., flow rate 1 ml/min.
tr=15 min (unresolved peak).
SEC: conditions no. 1 tr=40 min
2—The solid obtained above is dissolved in 200 ml of trifluoroacetic acid. After stirring at ambient temperature for 3 hours, the liquid is eliminated under reduced pressure and the residue is crystallized by adding diethyl ether. 8.8 g of white crystals, trifluoroacetate of the amine of formula VIII are thus obtained.
HPLC: column no. 1, eluent no. 7 linear gradient 98/2 to 60/40 (V/V) in 50 minutes, flow rate 1 ml/min.
tr=76 min (broad peak).

b) 131 mg of the hexaacid derived from cyclotriphosphazene, already used in Example 9, are introduced into 55 ml of dimethyl sulfoxide, along with 4.4 g of the solid obtained in step a) and 0.42 mg of triethylamine, and then 350 mg of EDCl and 250 mg of HOBT. After stirring at ambient temperature for 24 hours, the solution is introduced into 300 ml of ethanol; the precipitate then formed is dissolved in 200 ml of water and the solution is ultrafiltered through a polyethersulfone membrane with a cut-off threshold of 10 kD. After evaporation of the retentate, the residue in 50 ml of water is treated successively with an anionic resin in OH⁻ form and then a cationic resin, before eliminating the water.

The crystals thus isolated are a mixture of the expected product with that in which only 5 of the acid functions are amidated. The 2 products are separated by preparative HPLC.
HPLC: column no. 4: Zorbax 300 SB, C18, 100 Å, 5 μm;
L=25 cm; d=4.6 mm (Hewlett Packard®); eluent no. 7: linear gradient 95/5 to 90/10 (V/V) in 10 min and, after 5 min, 90/10 to 85/15 (V/V) in 5 min, then linear gradient up to 70/30 in 5 min
tr=17 min (broad peak)
(and tr=23 min for the pentaamidated product).
SEC: conditions no. 1 tr=35 min.

In the following examples, the SEC and HPLC conditions are as follows: SEC (steric exclusion chromatography): carried out on a succession of 4 columns (d=8 mm, L=30 cm) sold by Shodex® under the references OH Pack SB-HQ® containing polyhydroxymethacrylate gel, the exclusion limits of which, determined with Pullulan, are successively: 10⁶ KD (SB-804); 10⁵ KD (SB-803); 10⁵ KD (SB-803); 10⁴ KD (SB-802.5); eluent: aqueous NaCl solution (0.16M)/CH3CN 70/30 v/v, flow rate 0.8 ml/min T=30° C.

HPLC, Columns:

Zorbax® 300SB-CN, 5 μm, L=25 cm, d=4.6 mm (Interchim®)
Symmetry® C18, 100 Å, 5 μm, L=25 cm, d=4.6 mm (Waters®).
Lichrospher® RP-select B, 60 Å, 5 μm, L=12.5 cm, d=4 mm (Merck®).
Lichrospher® RP18, 100 Å, 12 μm, L=25 cm, d=50 mm (Merck®).
PLRP-S, 300 Å, 7 μm, L=15 cm, d=4.6 mm (Polymer Laboratories®).
Hypercarb® 100% C, 100 Å, 5 μm, L=25 cm, d=4.6 mm (Thermo Hypersil-Keystone®)
X-TerraMS® C18, 125 Å, 5 μm, L=25 cm, d=4.6 mm (Waters®)

### EXAMPLE 14 (MC 687)

Condensation of a Compound of Formula 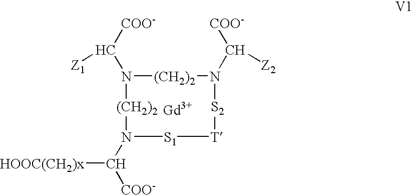
in which x=2 and —S₁-T′-S₂— is 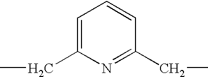
with S₁=S₂=CH₂
Z₂ is 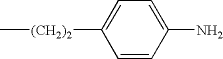
and Z₁ is (CH₂)₂COOH.

On 2,4,6-trichloro-1,3,5-triazine (cyanuryl chloride), 0.5 g of the compound of step e) of Example 3, are added to 2.5 ml of water. The product is solubilized by bringing the pH of the solution to 7 by adding a sodium hydroxide solution (0.1 N). The reaction medium is heated to 60° C. and a homogeneous solution made up of 0.029 g of 2,4,6-trichloro-1,3,5-triazine in 1 ml of dioxane is added in a single step.

The entire mixture is stirred at ambient temperature for 24 hours maintaining the pH of the solution at 8.4 by adding a sodium hydroxide solution (0.1 N). The reaction medium is ultrafiltered by centrifugation in 4 MICROSEP® tubes (1 KD) from FILTRON®. The retentate is concentrated under vacuum and dried at 50° C. in a ventilated oven in the presence of P₂O₅. 0.33 g is isolated with a crude yield of 84%.

Mass Spectrum:

Mode ES⁻ m/z=2468.40 with z=1

HPLC:

Symmetry® C18 column

Water-TFA, pH 3/CH₃CN

tr: Unresolved Peak, 15-17 min

### EXAMPLE 15 (MC 611): (PI 861)

Compound of Formula: II′₂ with x=2

-GNH— is

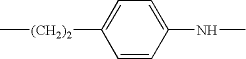

R is

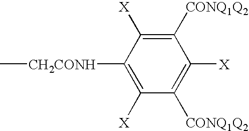

with Q₁=Q₂=CH₂(CHOH)₄CH₂OH and X=Br (branch referred to as AAG1 AA28 Br)
D−H=D′ being written: 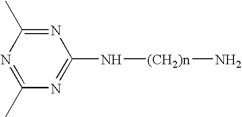
with n=3

It is explained here (the approach will be similar for the following examples) that: D of the compound II′2 is written: 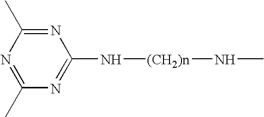
the compound MC611 obtained comprising D′=DH.

The compound MC611, referred to as type P730 (the Gd core is a core of DOTA type with substitututed alpha carbon) with branch AAG1 AA28 Br, has the formula below:

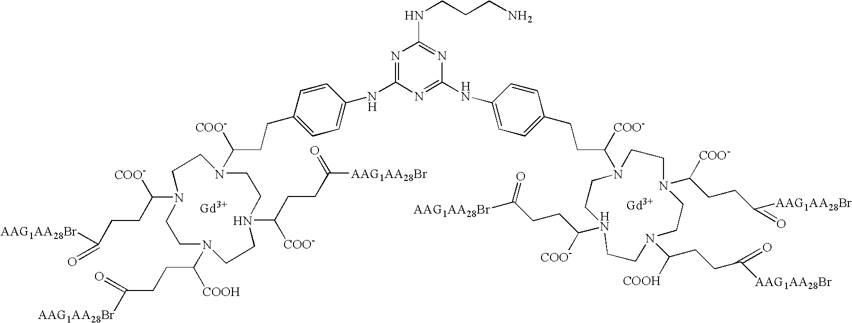

a) Condensation on the Triazine Ring

A solution of 0.66 g of 2,4,6-trichloro-1,3,5-triazine in 9 ml of dioxane is added, with stirring, to a solution of 7.6 g of the compound of step e) of Example 4 in 75 ml of water in the presence of NaHCO₃, of pH 7.7. After stirring at ambient temperature for 6 h, the reaction medium is stored overnight at 4° C.

Mass Spectrum:

Mode ES⁺ m/z=950 with z=2

HPLC:

Symmetry® C18 column

Water-TFA pH 3/CH₃CN

tr: 41 min.

b) Coupling of the Amine R—NH2

At ambient temperature and with vigorous stirring, 30.06 g of amine R—NH2, pH=6.65, are added to the solution of step a). 0.2 ml of 6N HCl is added so as to obtain a pH=6.2. 0.47 g of NHS and then 5.8 g of EDCl are subsequently added to the reaction medium. After stirring at ambient temperature for 3 h, a volume of water is added to the reaction medium and ultrafiltration is carried out through a polyethersulfone membrane (Pall®) with a cut-off threshold of 1 KD; the retentate is evaporated to a volume of 100 ml, and is then poured onto 1000 ml of EtOH under cold conditions. The precipitate formed is isolated: Mass obtained=29 g.

Mass Spectrum:

Mode ES⁻ m/z=2141.6 with z=4.

HPLC:

Symmetry® C18 column

Water-TFA pH 2.8/CH₃CN

tr: 22 min.

c) Introduction of the Diamine

29 g of intermediate of step b) are dissolved in 90 ml of diaminopropane at 50° C. After stirring for 1 h, the reaction medium is poured, under cold conditions, into 900 ml of ethanol. The precipitate formed is isolated, and dried under vacuum in the presence of P₂O₅. The product obtained is redissolved in 400 ml of water so as to be purified by ultrafiltration through a polyethersulfone membrane (Pall®) with a cut-off threshold of 1 KD. 22 g of white solid are isolated by elimination of the solvent from the retentate. The product is purified by preparative HPLC: Mass obtained=17 g.

HPLC:

Licrospher(E) RP18 column

Water/CH₃CN

tr=25 min (Broad Peak).

Mass Spectrum:

Mode ES⁻ m/z=8605 with z=1

### EXAMPLE 16 (MC 602): (PI854)

Compound of Formula: II′₂ with x=2

-GNH— is

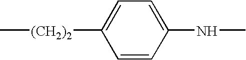

R is

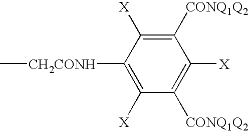

with Q₁=Q₂=CH₂(CHOH)₄CH₂OH and X=Br D′ is D-H 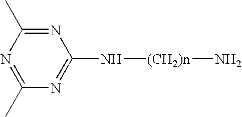
with n=2

a) Introduction of the Diamine

8 g of the intermediate b) of Example 15 are dissolved in 80 ml of water and 0.75 ml of diaminoethane, at 50° C. After stirring at 50° C. for 3 h 30 min, the reaction medium is poured into 800 ml of ethanol. The precipitate obtained is filtered, washed with ethanol and dried in a dessicator under vacuum in the presence of P₂O₅.

Mass Obtained=8 g.

HPLC:

Symmetry® C18 column

water-TFA pH 2.7/CH₃CN

tr=17.9 min.

Mass Spectrum

Mode ES⁻ m/z=2146.9 with z=4

### EXAMPLE 17 (MC 703): (PI933)

Compound of Formula: II′₂ with x=2

-GNH— is 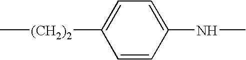
and R is 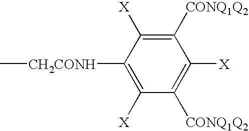
with Q₁=CH₂CHOHCH₂OH and Q₂=CH₂(CHOH)₄CH₂OH and x=Br (branch referred to as AAG1 AA29 Br)
D′ is D-H: 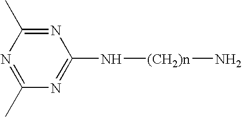
with n=3.

a) Coupling of the Amine R—NH2

20.02 g of the amine of formula RNH₂, 0.367 g of sodium salt of (N-hydroxysuccinimidyl)-3-sulfonic acid (NHS) and 4.54 g of 1-(3-dimethylaminopropyl)-3-ethylcarbodiimide hydrochloride (EDCl) are introduced into the water-dioxane solution containing 5.3 g of the compound obtained in step a) of Example 15. The mixture is stirred at 25° C. for 4 hours, during which time the pH is regularly brought to 6 by adding an aqueous N NaOH or HCl solution. The solution is poured into 800 ml of ethanol. The precipitate obtained is redissolved in 800 ml of water so as to be purified by ultrafiltration through a polyethersulfone membrane (Pall®) with a cut-off threshold of 1 KD. 20 g of solid are isolated by elimination of the solvent from the retentate.

Mass spectrum:

Mode ES⁻ m/z=1497.2 with z=5

HPLC:

Symmetry® C18 column

water-TFA pH 3/CH₃CN

tr=26 min.

b) Introduction of the Diamine

Starting with 19.85 g of the compound obtained in step a), 3.3 g of powder are obtained according to the process described in step c) of Example 15 and after purification by preparative HPLC.

Mass Spectrum:

Mode ES⁻ m/z=1503.9 with z=5

HPLC:

Symmetry® C18 column

water-TFA pH 3/CH₃CN

tr=20 min.

### EXAMPLE 18 (MC 712): (PI938)

Compound of Formula: II′₂ with x=2

-GNH— is 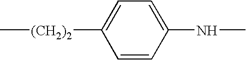
and R is a “flash” branch:

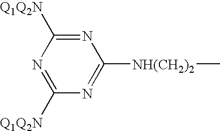

with Q₁=Q₂=CH₂(CHOH)₄CH₂OH
D′ is D-H: 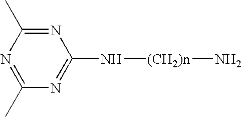
with n=3.

a) Reaction of the amine Q₁Q₂NH (6-[(2,3,4,5,6-pentahydroxyhexyl)amino]-1,2,3,4,5-hexanepentol) on 2,4,6-trichloro-1,3,5-triazine (cyanuryl chloride).

In a 1 L round-bottomed flask, 103.65 g (0.3 mol) of amine Q₁Q₂NH are introduced into a suspension of 27.6 g (0.15 mol) of cyanuryl chloride in 300 ml of water. The pH is adjusted to 7.5-8 with a 5 N sodium hydroxide solution. The suspension is stirred overnight at ambient temperature.

HPLC:

Hypercarb® column

water-H₂SO₄ 0.037 N/CH₃CN

tr=27 min.

b) Condensation of Ethylenediamine

200.5 ml of ethylenediamine (3 mol) are-then added to the reaction mixture. Stirring is maintained for 1 h30 and the reaction medium is then concentrated under vacuum. 250 ml of water are added to the oil obtained above and the mixture is then again concentrated under vacuum, thus eliminating a portion of the excess ethylenediamine. The crude product is purified on 3 kg of silanized silica (elution with water). 155.3 g of product are thus obtained.

HPLC:

Hypercarb® column

water-H₂SO₄ 0.037 N/CH₃CN

tr=20 min.

Mass Spectrum:

Mode ES⁺ m/z=826.7 with z=1.

c) Coupling of the Amine R—NH2

By applying the same procedure as for Example 15, step b), 20.4 g of solid are obtained by starting with 5.3 g of compound a) of Example 15 and 17.7 g of amine RNH₂ prepared in step b), and then eliminating the low molecular weight impurities.

HPLC:

Symmetry® C18 column

water-TFA pH 3 CH₃CN

tr=18-20 min.

Mass Spectrum:

Mode ES⁻ m/z=1348.5 with z=5.

d) Introduction of the Diamine

By applying the same procedure as for the process described in step c) of Example 15, 5 g of solid are obtained from 19.4 g of the compound obtained in the preceding step c) and 60 ml of diaminopropane.

HPLC:

Symmetry® C18 column

water-TFA pH 3/CH₃CN

tr=21-30 min.

Mass Spectrum:

Mode ES⁻ m/z=1132.0 with z=6.

### EXAMPLE 19 (MC 708): (PI942)

Compound of Formula: II′₂ with x=2

-GNH— is 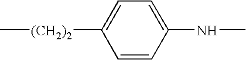
and R is 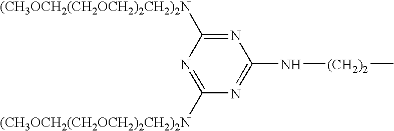
D′ is D-H: 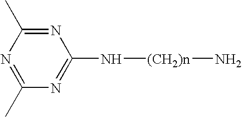
with n=3.

a) Tosylation

A solution of 251.6 g of tosyl chloride in 260 ml of pyridine is added dropwise, at +5° C., to a solution of 108 g of triethylene glycol monomethyl ether in 160 ml of pyridine, cooled to 0° C. with an ice-acetone bath. The mixture obtained is stirred for 12 hours at ambient temperature, and then poured into 500 ml of cold water and extracted with 2 times 500 ml of ethyl ether. After concentration of the organic phase, 204 g of product are obtained.

TLC:

Eluent: CH₂Cl₂ 90%-MeOH 10%-SiO₂

Visualizing means: UV

Rf: 0.6.

b) Condensation on Benzylamine

A solution of 199 g of the intermediate obtained in the preceding step a) dissolved in 800 ml of acetonitrile is added dropwise at ambient temperature to a solution made up of 31 ml of benzylamine and 66.3 g (0.624 mol) of Na₂CO₃ dissolved in 400 ml of CH₃CN. The medium is stirred at reflux for 24 hours. After filtration of the insoluble material, the medium is concentrated to dryness. 111 g of product are obtained.

TLC:

Eluent: CH₂Cl₂ 90%-MeOH 10%-SiO₂

Visualizing means: UV

Rf: 0.8.

c) Deprotection of the Amine

70 g (0.175 mole) of product derived from step b) are introduced, in the presence of 470 ml of ethanol and of 15 g of Pd-on-charcoal at 10%, into a l-liter autoclave. The medium is hydrogenated under 12 bar at 45° C. for 6 hours. After filtration and concentration, 50 g of product are obtained.

Mass Spectrum:

Mode ES⁺ m/z=309.9 with z=1

TLC:

Eluent: CH₂Cl₂ 90%-MeOH 10%-SiO₂

Visualizing means: Dragendorff reagent

Rf: 0.25.

d) Condensation on 2,4,6-trichloro-1,3,5-triazine

A solution made up of 50 g of product obtained in step c), 49.5 g of potassium carbonate, 14.93 g of cyanuryl chloride dissolved in 1.5 liters of 1,4-dioxane is stirred at 42° C. for 48 hours. After filtration of the medium and concentration, 56 g of product are obtained (yield 94%).

Mass Spectrum:

Mode ES⁺ m/z=730.4 with z=1

HPLC:

Symmetry® C18 column

water-TFA pH 3.16/CH₃CN

tr=37.7 min.

e) Condensation of the Amine

A solution made of up 56 g of compound obtained in step d), 25.75 ml of ethylenediamine and 21.3 g of potassium carbonate in 1.1 liters of 1,4-dioxane is stirred at 42° C. for 24 hours. The product is purified by chromatography on silica, elution being carried out with a mixture made up of 95% CH₂Cl₂/5% MeOH. 30 g of product are obtained.

Mass Spectrum:

Mode ES⁺ m/z=377.4 with z=2

HPLC:

Symmetry® C18 column

water-TFA pH 3.20/CH₃CN

tr=16.95 min.

f) Coupling of the Amine R—NH2

The coupling is carried out according to the protocol described in step b) of Example 15, starting with 17.5 g of the amine R—NH₂ prepared above, dissolved in a minimum amount of water. The reaction medium is diluted in 300 ml of water so as to be ultrafiltered through a 1 KD polyethersulfone membrane (Pall®). The retentate is evaporated to dryness. 15 g of oil are obtained.

Mass Spectrum:

Mode ES⁺ m/z=6316.15 with z=1

HPLC:

Symmetry® C18 column

water-TFA pH 2.7/CH₃CN

tr=21 min.

g) Introduction of the Diamine

Starting with 2.4 g of the intermediate prepared above, 3.4 g of product are obtained according to the protocol described in step c) of Example 15, after evaporation of the solvent and purification of the oil obtained by preparative HPLC.

Mass Spectrum:

Mode ES⁺ m/z=6352.7 with z=1

HPLC:

Symmetry® C18 column

water-TFA pH 2.8/CH₃CN

tr=16.5 min.

### EXAMPLE 20 (MC 680): (PI879)

Compound of Formula: II′₂ with x=2

-GNH— is —(—CH₂)₃—NH₋

R is

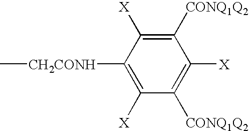

with Q₁=Q₂=CH₂(CHOH)₄CH₂OH and X=Br
D′ is D-H 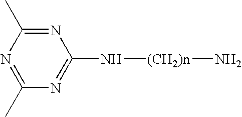
with n=3.

a) Condensation on the Triazine Ring

is Starting with 0.2 g of amine obtained in Example 5 d) and 19.3 mg of 2,4,6-trichloro-1,3,5-triazine, 0.18 g of product obtained according to the protocol described in Example 15 a).

Mass Spectrum:

Mode ES m/z 1777 with z 1

HPLC:

Symmetry® C18 column

water-TFA pH 2.8/CH₃CN

tr=30,31,32 min.

b) Coupling of the Amine R—NH2

By applying the protocol described in Example 15 b), 0.46 g of amine R—NH₂ (0.41 mmol) and 0.1 g (0.056 mmol) of the intermediate a) prepared above produce 0.25 g of product.

Mass Spectrum:

Mode ES⁻ m/z=8442 with z=1

HPLC:

Symmetry® C18 column

water-TFA pH 2.8/CH₃CN

tr=16.5 min.

c) Introduction of the Diamine

By applying the protocol of Example 15 c) to 0.2 g of derivative prepared above b), 0.1 g of product is obtained.

Mass Spectrum:

Mode ES⁻ m/z=8483 with z=1

HPLC:

Symmetry® C18 column

water-TFA pH 2.8/CH₃CN

tr=14.4 min.

### EXAMPLE 21 (MC 691): (PI932)

Compound of Formula: II″ₐ₂ with x=2

-GNH— is

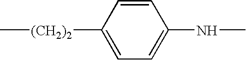

R is

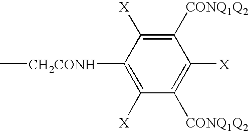

with Q₁=CH₂CHOHCH₂OH and Q₂=CH₂(CHOH)₄CH₂OH, X=Br

D′is D-H:

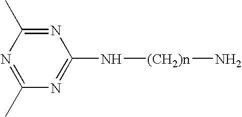

with n=3

a) Condensation of the Triazine Ring

5 g of the compound obtained in step e) of Example 3 are condensed on 2,4,6-trichloro-1,3,5-triazine according to the protocol described in step a) of Example 15. After reaction for 3 h, the pH is brought back to 7 with a sodium hydrogen carbonate solution. The solution obtained is stored in a refrigerator overnight.

Mass Spectrum:

Mode ES⁻ m/z=852.7 with z=2

HPLC:

Symmetry® C18 column

water-TFA pH 2.95 CH₃CN

tr=20-25 min.

b) Coupling of the Amine R—NH2

12.5 g of the amine R—NH₂ are added to the solution containing the compound prepared in step a). The peptide coupling is carried out according to the protocol described in step a) of Example 17. 12.61 g of product are isolated.

Mass Spectrum:

Mode ES⁻ m/z=1810.2 with z=3

HPLC:

Symmetry® C18 column

water-TFA pH 2.90/CH₃CN

tr=10-16 min.

c) Introduction of the Diamine

12 g of the product prepared in step b) are solubilized with 90 ml of 1,3-diaminopropane. Using the protocol described in step c) of Example 15, 7.5 g of product are isolated with a yield of 62%.

Mass Spectrum:

Mode ES⁻ m/z=1822.6 with z=3

HPLC:

X-TERRA MS® column

water/CH₃CN

tr=9-13 min.

### EXAMPLE 22 (MC 768): (PI955)

Compound of Formula: II″ₐ₂ with x=2

-GNH— is

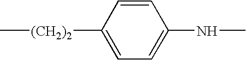

R is

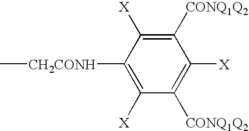

with Q₁=Q₂=CH₂(CHOH)₄CH₂OH and X=Br

D′ is D-H

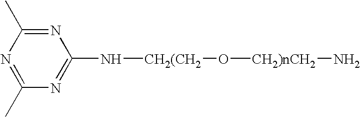

with n=2

a) Coupling of the Amine R—NH2

14 g of the amine of formula RNH₂, 2.68 g of 1-(3-dimethylaminopropyl)-3-ethyl-carbodiimide (EDCl) and 0.326 g of sodium salt of N-(hydroxysuccinimidyl)-3-sulfonic acid (NHS) are added to the water-dioxane solution containing 4.2 g of the compound obtained in step a) of,Example 21. Coupling is carried out according to the protocol described in step b) of Example 15, so as to obtain 18 g of product.

Mass Spectrum:

Mode ES⁻ m/z=1024.9 with z=6

HPLC:

Symmetry® C18 column

water-TFA pH 2.7/CH₃CN

tr=13 min.

b) Introduction of the Diamine

20 ml of 2-[2-(2-aminoethoxy)ethoxy]ethylamine are added to a solution of 70 ml of dimethyl sulfoxide containing 18 g of the compound obtained in step a). The mixture is stirred at 50° C. for 1 hour. After cooling to 25° C., the solution is poured into 1000 ml of ethanol, the precipitate formed is dissolved in 400 ml of water and the solution is ultrafiltered through a polyethersulfone membrane (Pall®) with a cut-off threshold of 1 KD. After evaporation of the retentate, the product obtained is purified by preparative HPLC. 1.5 g of solid are thus obtained.

Mass Spectrum:

Mode ES⁻ m/z=2087.2 with z=3

HPLC:

Symmetry® C18 column

water-TFA pH 2.7/CH₃CN

tr=11 min.

### EXAMPLE 23 (MC 695): (PI953)

Compound of Formula: II′″₂ with x=2

-GNH— is —(—CH₂)₃—NH₋

R is

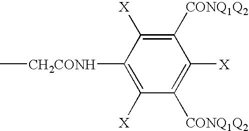

with Q₁=CH₂CHOHCH₂OH and Q₂=CH₂(CHOH)₄CH₂OH, X=Br
D′ is D-H

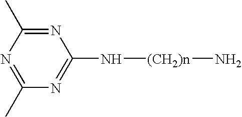

with n=3.

a) Condensation on the Triazine Ring

0.324 g of potassium carbonate, and 0.190 g of 2,4,6-trichloro-1,3,5-triazine in 11 ml of dioxane are added to a solution containing 8.5 g of the compound obtained according to step b) of Example 6 in 56 ml of distilled water, and the pH is then brought to 8.4 by adding K₂CO₃. The reaction medium is stirred for 18 h, during which time the pH is brought to 8.4 by adding K₂CO₃. After neutralization with cationic resin in H⁺ form, the solvents are evaporated off and the residue is taken up in absolute ethanol. The precipitate is isolated: m=8.3 g

Mass Spectrum:

Mode ES⁻ m/z=2437 with z=3

HPLC:

Symmetry® C18® column

water/CH₃CN

tr 13.3 min.

b) Introduction of the Diamine

0.317 g of potassium carbonate and 1.14 ml of diaminopropane are added to a solution containing 8.3 g of the compound obtained in step a) in 70 ml of dimethyl sulfoxide. The medium is stirred at 42° C. for 18 h under argon. The mixture is poured into 300 ml of ethanol and the precipitate formed is filtered and then washed with ethyl ether, filter-dried and dried. After purification by preparative HPLC and ultrafiltration through a polyethersulfone membrane (Pall®) with a cut-off threshold of 3 KD, 3.9 g of crystals are obtained after evaporation of the water.

Mass Spectrum:

Mode ES⁻ m/z=1469.8 with z=5

HPLC:

Lichrospher C18® column

water-TFA pH 3.3/CH₃CN

tr=16.5 min.

### EXAMPLE 24 (MC 766):

Compound of Formula: II′″₂ with x=2

-GNH— is —(—CH₂)₃—NH

R is

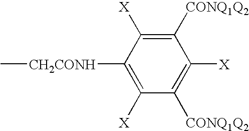

with Q₁=Q₂=CH₂(CHOH)₄CH₂OH and X=Br
D′ is D-H 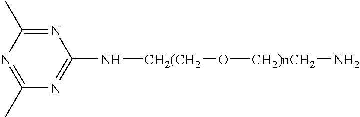
with n=2.

a) Condensation on the Triazine Ring

0.132 g of potassium carbonate is added to a solution containing 4 g of the compound obtained according to step a) of Example 13 in 25 ml of distilled water. A solution of 0.080 g of 2,4,6-trichloro-1,3,5-triazine in 5.5 ml of dioxane is added and the pH is then brought to 8.4 by adding K₂CO₃. 3.7 g of product are isolated following the protocol of Example 23 a).

Mass Spectrum:

Mode ES⁻ m/z=2099 with z=4

HPLC:

Symmetry® C18 column

water/CH₃CN

tr=10.8 min.

b) Introduction of the Diamine

Starting with a solution containing 3.7 9 of the compound obtained in step a) in 30 ml of dimethyl sulfoxide, 0.220 g of potassium carbonate and 1.3 g of diamine 2-[2-(2-aminoethoxy)ethoxy]ethylamine are added according to the protocol described in step b) of Example 23. After purification by preparative HPLC, 1 g of product is obtained.

Mass Spectrum:

Mode ES⁻ m/z=1700.3 with z=5

HPLC:

Lichrospher C18® column

water-TFA pH 3.3/CH₃CN

tr=13.8 min.

### EXAMPLE 25 (MC 723) P934

Compound of Formula: I₂ with

W₂: 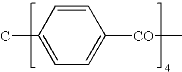
A₂ is II′₂ with x=2
G-NH, R and D′=D-H as defined in Example 17.
m₂=4

116 microliters of triethylamine, 0.088 g of 1-(3-dimethylaminopropyl)-3-ethyl-carbodiimide hydrochloride (EDCl) and 0.062 g of hydroxy-1-benzotriazole hydrate are added to a solution of 8 ml of dimethyl sulfoxide containing 0.041 g of tetrakis (4-carboxyphenyl)methane and 3.1 g of the compound MC703 obtained in step b) of Example 17, at 40° C. After stirring at 40° C. for 18 hours, the solution is poured into 80 ml of ethanol and the precipitate obtained is separated by filtration and then redissolved in 100 ml of water. The solution is ultrafiltered through a membrane (Millipore®) with a cut-off threshold of 1.0 KD. After evaporation of the water from the retentate, 2.3 g of product are obtained.

Mass Spectrum:

Mode ES⁻ m/z=2773.3 with z=11

SEC:

Columns OH Pack SB-HQ® SB-804, 2×SB-803, SB-802.5

Water-0.16 M NaCl/CH₃CN, 70/30

tr=34.6 min.

The compound MC 723 has the formula:

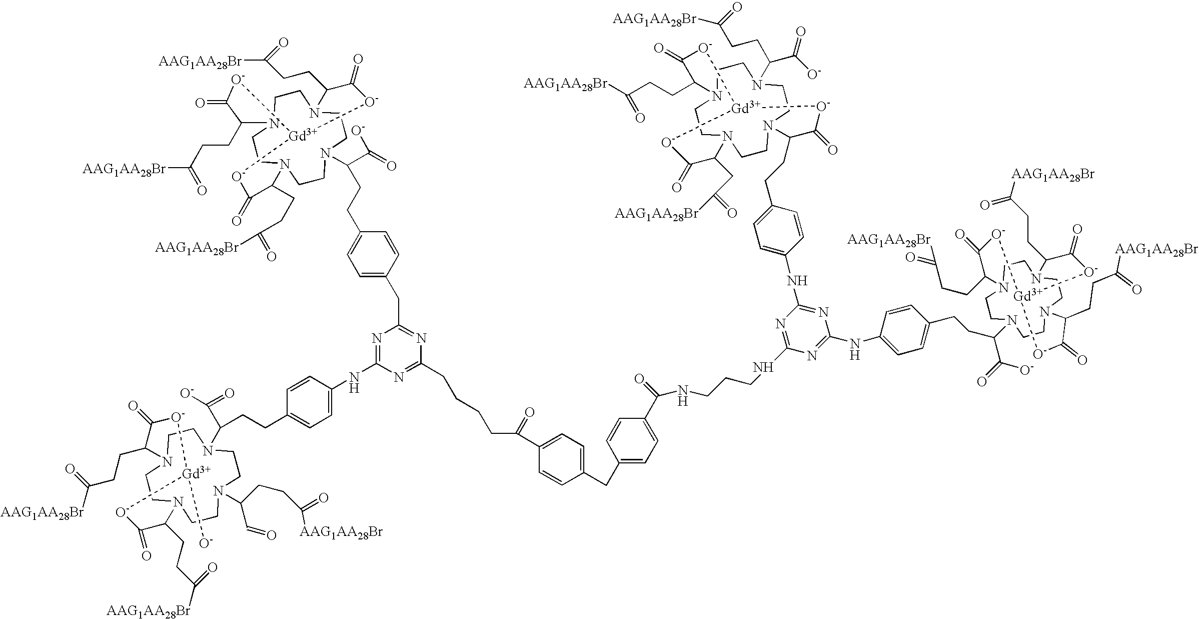

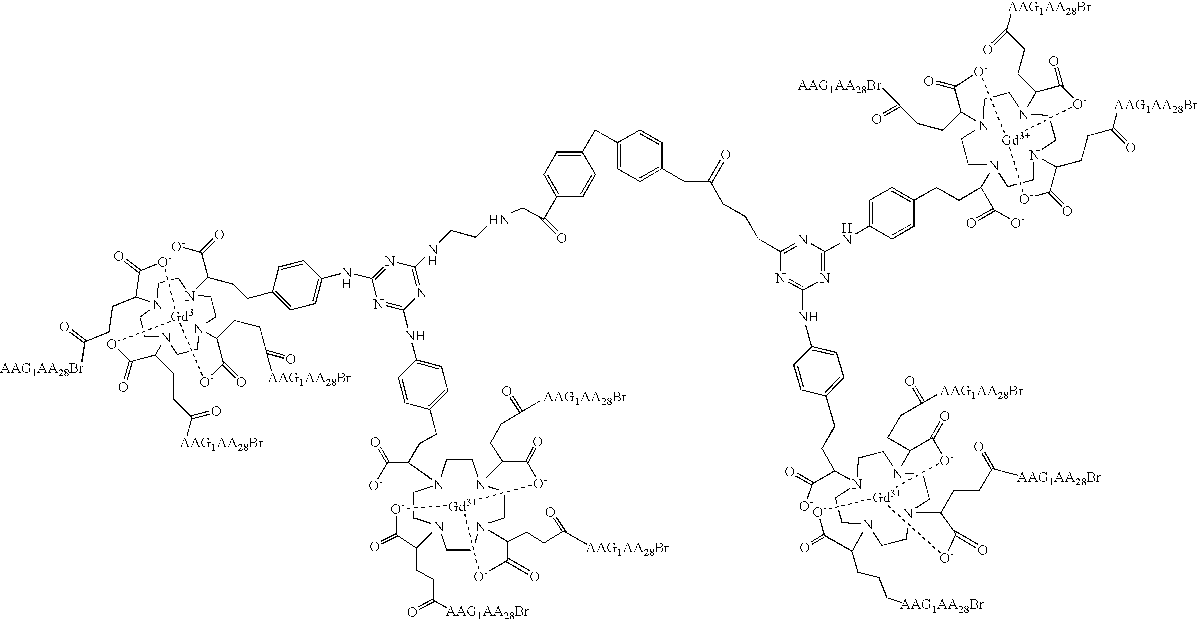

### EXAMPLE 26 (MC 716) P928

Compound of Formula: I₂ with

W₂: 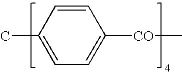
A₂ is II′₂ with x=2
G-NH, R and D′=D-H as defined in Example 19.
m₂=4

2.7 g of intermediate g) of Example 19 (MC708) and 50 mg of tetrakis(4-carboxyphenyl)methane intermediate are dissolved in 6 ml of DMSO at 80° C. The coupling is carried out according to the protocol described in Example 25. After ultrafiltration and evaporation, 1.3 g of amber gum are obtained.

Mass Spectrum:

Mode ES⁻ m/z=25835 with z=1

SEC:

Columns OH Pack SB-HQ® SB-804, 2×SB-803, SB-802.5

Water-NaCl 0.16M/CH₃CN 70/30

tr=35.25 min.

HPLC:

Symmetry® C18 column

eau-TFA pH 2.8/CH₃CN

tr=37.8 min.

### EXAMPLE 27 (MC 755) P944

Compound of Formula: I₂ with

W₂: 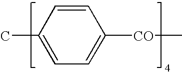
A₂ is II′₂ with x=2
G-NH, R and D′=D-H as defined in Example 18.

m₂=4

By applying the same procedure as for Example 25, 0.938 g of crystals are obtained from 1.87 g of the compound obtained in step d) of Example 18 and from 0.028 g of tetrakis (4-carboxyphenyl)methane.

Mass Spectrum:

Mode ES⁻ m/z=3442.5 with z=8

SEC:

Columns OH Pack SB-HQ® SB-804, 2×SB-803, SB-802.5

Water-NaCl 0.16M/CH₃CN 70/30

tr=35min.

### EXAMPLE 28 (MC 606) P855

Compound of Formula: I₂ with

W₂: 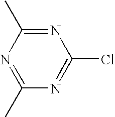
A₂ is II′₂ with x=2
G-NH, R and D′=D-H as defined in Example 16.
m₂=2

The compound prepared in Example 16 (MC602) is dissolved in 1 ml of water in the presence of 9.2 mg of NaHCO₃ so as to have a pH=7. A cyanuryl chloride solution is prepared from 0.32 g dissolved in 10 ml of dioxane. 100 μl of solution are taken therefrom, so as to add it to the reaction medium at ambient temperature. After stirring overnight, the reaction medium is diluted in 75 ml of water and then ultrafiltered through a 10 KD polyethersulfone membrane (Pall®). After evaporation of the water, 150 mg of white crystals are obtained.

Mass Spectrum:

Mode ES⁻ m/z=17297 with z=1

HPLC:

Symmetry® C18 column

water-TFA pH 2.8/CH₃CN

tr=21 min.

### EXAMPLE 29 (MC 607) P856

Compound of Formula: I₂ with

W₂: 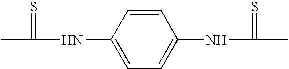
A₂ is II′₂ with x=2
G-NH, R and D′=D-H as defined in Example 16.
m₂=2.

The compound prepared in Example 16 (MC602) is dissolved in 1 ml of water in the presence of NaHCO₃ so as to have a pH=6. A solution of phenyl 1,4-diisothiocyanate is prepared from 330 mg of product dissolved in 10 ml of dioxane. 100 μl of this solution are added to the reaction medium at 50° C. After stirring for 18 h, the reaction medium is diluted in 50 ml of water and then ultrafiltered through a 10 KD polyethersulfone membrane (Pall®).

After evaporation of the water, 80 mg of white crystals are obtained.

Mass Spectrum:

Mode ES⁻ m/z=17387.47 with z=1

HPLC:

Symmetry® C18 column

water-TFA pH 2.8/CH₃CN

tr=21.9 min.

### EXAMPLE 30 (MC 616) P858

Compound of Formula: I₂ with W₂: 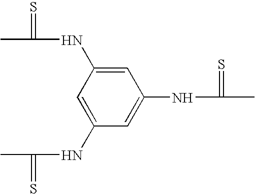
A₂ is II′₂ with x=2
G-NH, R and D′=D-H as defined in Example 15.
m₂=3.

a) Catalytic Reduction

5 g of amino-3,5-dinitrobenzene are dissolved in 150 ml of MeOH in the presence of 0.5 g of palladium-on-charcoal at 5% under a hydrogen pressure of 2 Atm. After reaction at ambient temperature for 6 h, the reaction medium is filtered through paper, evaporated and suspended in 25 ml of d'Et₂O. After filtration, 2.9 g of black crystals are obtained.

TLC:

Eluent: CH₂Cl₂8/MeOH 2—SiO₂

Visualizing means: UV

Rf: 0.15;

b) Thiophosgenation

0.5 g of Intermediate prepared in step a) is dissolved in 45 ml of CH₃CN and 5 ml of MeOH. 2 ml of thiophosgene are introduced rapidly and with vigorous stirring, at −5° C. After reaction at ambient temperature for 20 min, the reaction medium is poured into 100 ml of 0.5 M NaH₂PO₄ and 50 ml of Et₂O. The ethereal phase is dried over MgSO₄, filtered and evaporated. The oil obtained is purified by flash chromatography on silica (Merck®, 40-60 μm), elution being carried out with heptane/AcOEt (90/10, v/v). After elimination of the solvent, 0.2 g of the solid product is obtained.

TLC:

Eluent: 8 heptane/2 AcOEt—SiO₂

Visualizing means: UV

Rf: 0.5

HPLC:

Symmetry® C18 column

water-TFA, pH 2.8/CH₃CN

tr=26 min.

c) Condensation on the Central Nucleus

3 g of intermediate described in Example 15 c) (MC611) are dissolved in 6 ml of water at 50° C. The pH of the solution obtained is adjusted to 7.8 with NaHCO₃. A solution of 27.9 mg of intermediate prepared in step c) in 3 ml of dioxane is added. After reaction at 50° C. for 5 h, the reaction medium is cooled, diluted in 100 ml of water, and purified by ultrafiltration through a polyethersulfone membrane (Pall®) with a cut-off threshold of 10 KD.

After evaporation of the water, 1.8 g of white crystals are obtained.

Mass Spectrum:

Mode ES⁻ m/z=26035.6 with z=1

SEC:

Columns OH Pack SB-HQ® SB-804, 2×SB-803, SB-802.5

Water-NaCl 0.16M/CH₃CN 70/30

tr=35min.

### EXAMPLE 31 (MC 651) P870

Compound of Formula: I₂ with

W₂: 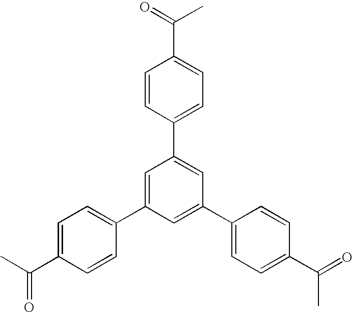
A₂ is II′₂ with x=2

G-NH, R and D′=D-H as defined in Example 15.

m₂=3.

47.6 mg of tris-1,3,5-(4-carboxyphenyl)benzene intermediate and 3 g of intermediate described in Example 15 c) (MC611) are dissolved in 6 ml of DMSO at 80° C. 61.4 mg of HOBT (1-hydroxybenzotriazole) and 87.4 mg of EDCl (1-(3-dimethylaminopropyl)-3-ethylcarbodiimide hydrochloride) are added at ambient temperature. After reaction at 40° C. for 20 h, the reaction medium is poured into 90 ml of ethanol. The precipitate obtained is filtered, washed, and dried in a dessicator under vacuum. The product obtained is redissolved in 100 ml of water so as to be ultrafiltered through a polyethersulfone membrane (Pall®) with a cut-off threshold of 10 KD and then of 30 KD. After evaporation of the water, 1.1 g of white crystals are obtained.

Mass Spectrum:

Mode ES⁻ m/z=26199 with z=1.

SEC:

Columns OH Pack SB-HQ® SB-804, 2×SB-803, SB-802.5

Water-NaCl 0.16M/CH₃CN 70/30

tr=35 min.

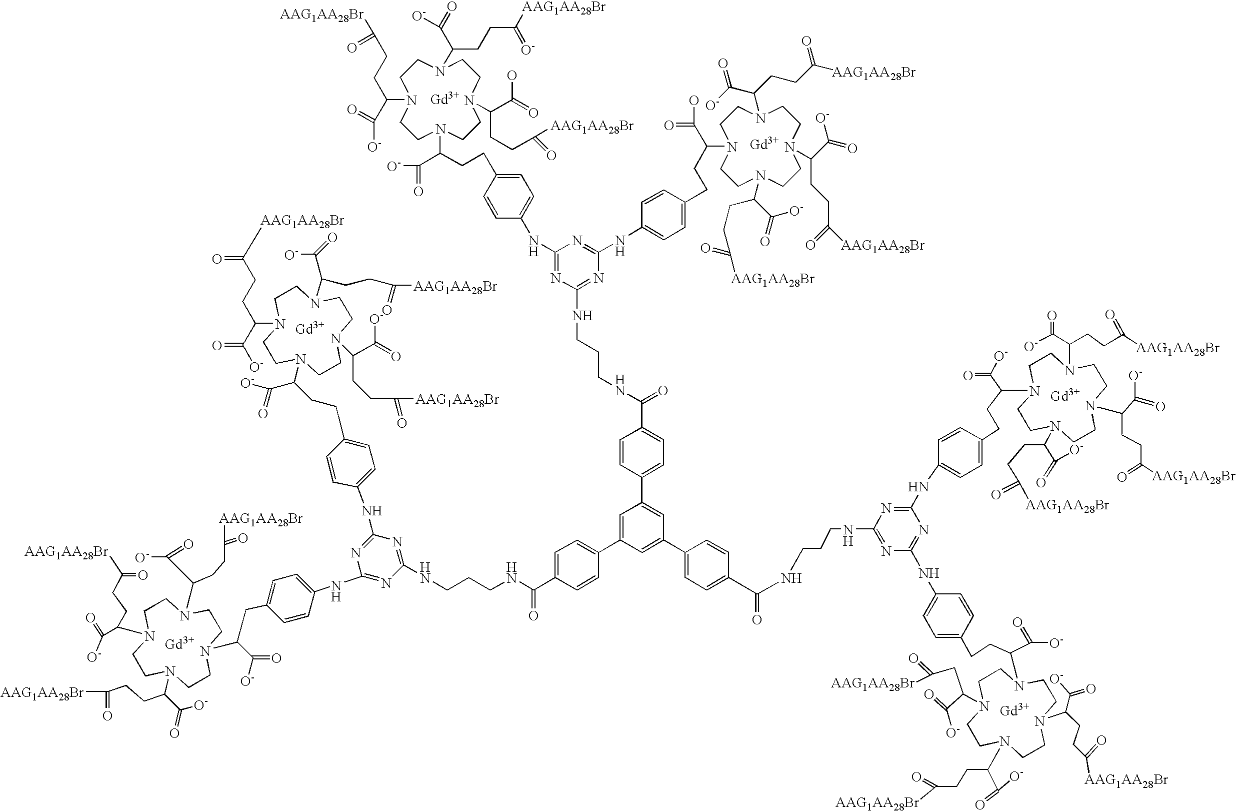

### EXAMPLE 32 (MC 635) P898

Compound of Formula: I₂ with

W₂: 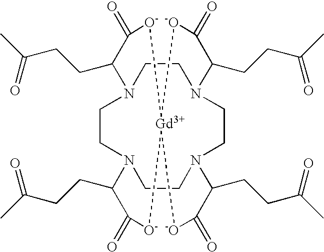
A₂ is II′₂ with x=2
G-NH, R and D′=D-H as defined in Example 15.
m₂=4.

26 mg of P730 2-[4,7,10-tris(1,3-dicarboxypropyl)-1,4,7,10-tetraazacyclo-dodecan-1-yl]pentanedioic acid) and 1 g of intermediate described in Example 15 c) (MC611) are dissolved in 2 ml of water. The pH of the solution obtained is adjusted to 6.2 with 0.1N HCl 4.8 mg of (N-hydroxysuccimidinyl)-3-sulfonic acid (NHS) and 29.6 mg of EDCl (1-(3-dimethylaminopropyl)-3-ethylcarbodiimide hydrochloride) are added. After reaction at 40° C. for 48 h, the reaction medium is diluted in 150 ml of water and purified by ultrafiltration through a polyethersulfone membrane (Pall®) with a cut-off threshold of 10 KD. After evaporation of the water, 500 mg of white crystals are obtained.

Mass Spectrum:

Mode ES⁻ 35201.82 with z=1.

SEC:

Columns OH Pack SB-HQ® SB-804, 2×SB-803, SB-802.5

Water-NaCl 0.16M/CH₃CN 70/30

tr=31 min.

### EXAMPLE 33 (MC 636) P865

Compound of Formula: 12 with

W₂: 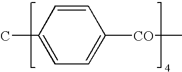
A₂ is II′₂ with, x=2

G-NH, R and D′=D-H as defined in Example 15.
m₂=4.

4.3 g of intermediate described in Example 15 c) (MC611) are dissolved in 10 ml of DMSO with 58.48 mg of tetrakis (4-carboxyphenyl)methane, at 80° C. The mixture is allowed to return to ambient temperature, before adding 172 μl of triethylamine, 89.87 mg of 1-hydroxybenzotriazole (HOBT) and: 127.26 mg of 1-(3-dimethylaminopropyl)-3-ethylcarbodiimide hydrochloride (EDCl). After stirring at 40° C. for 3 h, the reaction medium is cooled and diluted in 100 ml of water. The solution is ultrafiltered through a polyethersulfone membrane (Pall®) with a cut-off threshold of 10 KD and then of 30 KD. After evaporation of the water, 1.5 g of white crystals are obtained.

Mass Spectrum:

Mode ES⁻ 34855.94 with z=1.

SEC:

Columns QH Pack SB-HQ® SB-804, 2×SB-803, SB-802.5

Water-NaCl 0.16M/CH₃CN 70/30

tr=34.7 min.

### EXAMPLE 34 (MC 647)

Compound of formula: I₂ with

W₂: 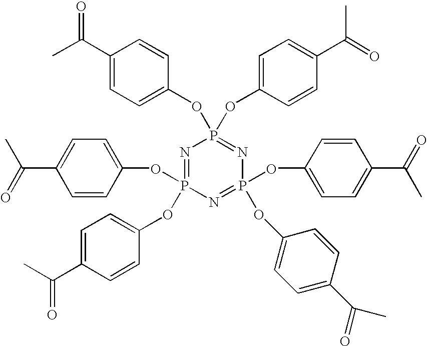
A₂ is II′₂ with x=2.
G-NH, R and D′=D-H as defined in Example 1.5.
m₂=6.

1.0 g of intermediate described in Example 15 c) (MC611) and 17.6 mg of 4-{[2,4,4,6,6-pentakis(4-carboxyphenoxy)-1,3,5,2λ⁵,4λ⁵,6λ⁵-triazatriphosphinin-2yl]oxy}benzoic acid are dissolved at 80° C. in 1 ml of DMSO. 40 μl of triethylamine, 20.9 mg of N-hydroxysuccinimide (NHS) and 29.6 mg of EDCl (1-(3-dimethylaminopropyl)-3-ethylcarbodiimide hydrochloride) are added, at ambient temperature. After reaction at 40° C. for 18 h, the reaction medium is diluted in 150 ml of water and purified by ultrafiltration through a polyethersulfone membrane (Pall®) with a cut-off threshold of 30 KD. After evaporation of the water, 0.7 g of white crystals is obtained.

Mass Spectrum:

Mode ES⁻ m/z=52527 with z=1

SEC:

Columns OH Pack SB-HQ® SB-804, 2×SB-803, SB-802.5

Water-NaCl 0.16M/CH₃CN 70/30

tr=33.4 min.

### EXAMPLE 35 (MC 711) P 949

Compound of Formula: I₂ with

W₂: 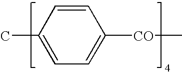
A₂ is II″ₐ₂ with x=2
G-NH, R and D′=D-H as defined in Example 21.
m₂=4
of formula (N-functionalized PCTA compound):

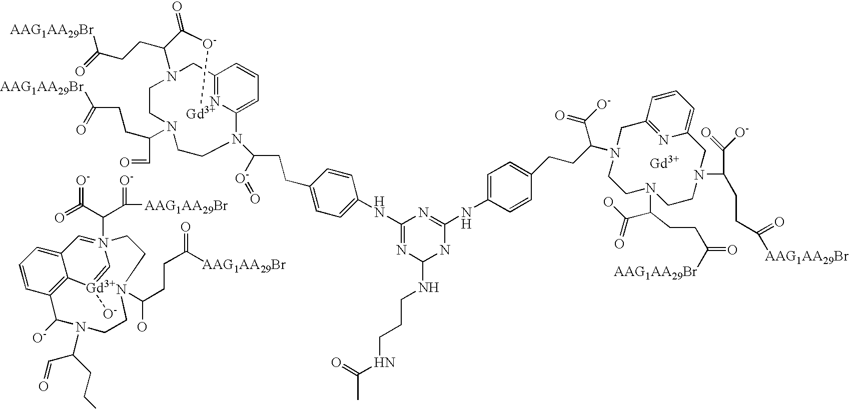

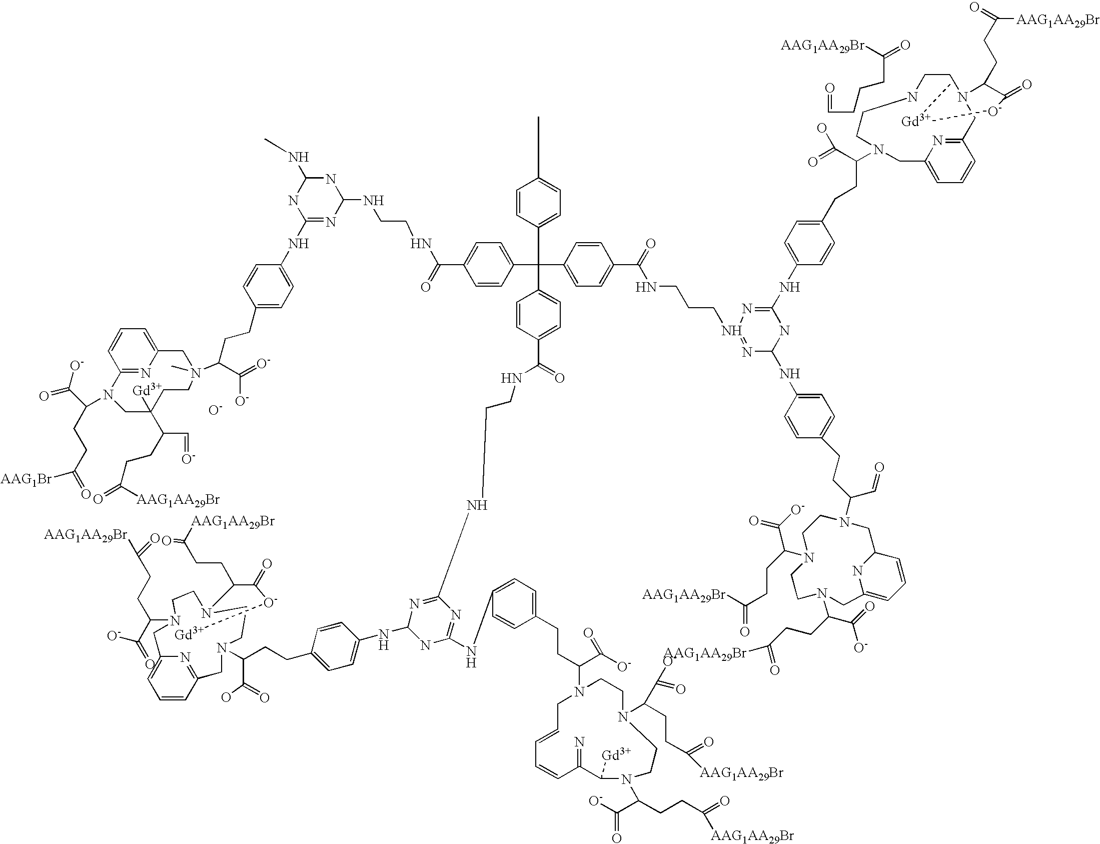

By applying the protocol described in Example 33, starting with 0.2 g of the compound obtained in Example 21 c) and 4.1 mg of tetrakis(4-carboxyphenyl)methane, 0.18 g of product is isolated.

Mass Spectrum:

Mode ES⁻ m/z=1858.5 with z=12

HPLC:

Column PLRP-S®

water-TFA pH 3.0/CH₃CN

tr=21-26 min.

### EXAMPLE 36 (MC 718) P 943

Compound of Formula: I₂ with

W₂: 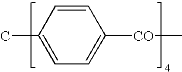
A₂ is II′″₂ with x=2
G-NH, R and D′=D-H as defined in Example 23.
m₂=4.

0.121 g of EDCl and 0.086 g of 1-hydroxybenzotriazole (HOBT) are added to a solution of 14.3 ml of dimethylacétamide containing 0.0393 g of tetrakis(4-carboxyphenyl)methane and 2.7 g of the compound obtained in Example 23 b). After 20 h at 45° C. the solution is poured into 100 ml of C₂H₅OH and the precipitate obtained is separated by filtration and then redissolved in 40 ml of water. The solution is ultrafiltered through a polyethersulfone membrane (Pall®) with a cut-off threshold of 10 KD. After evaporation of the water, 1.7 g of crystals are isolated.

Mass Spectrum:

Mode ES⁻ m/z=3726 with z=8

HPLC:

Column ZORBAX 300SB-CN®

CH₃COONH₄ 100 mM/CH₃CN

tr=57 min.

### EXAMPLE 37 (MC 797)

Compound of Formula: II″ₐ₂ with x=2

-GNH— is

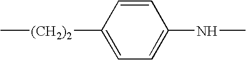

R is

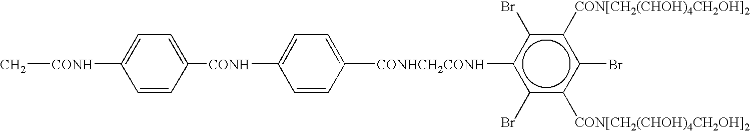

D′ is D-H:

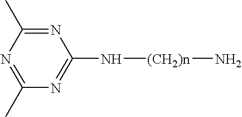

with n=3.

a) Coupling of the Amine R—NH₂

10.28 g of the amine of formula RNH₂, 1.61 g of 1-(3-dimethylaminopropyl)-3-ethylcarbodiimide hydrochloride (EDCl) and 0.196 g of sodium salt of (N-hydroxysuccinimidyl)-3-sulfonic acid (NHS) are added to the water-dioxane solution containing 2.56 g of the compound obtained in step a) of Example 21. The coupling is carried out according to the process described in step b) of Example 15, so as to obtain 12 g of product.

Mass Spectrum:

Mode ES⁻ m/z=1220.7 with z=6

HPLC:

Symmetry® C18 column

water-TFA pH 2.9/CH₃CN

tr=18.6 min.

b) Introduction of the Diamine

6 g of the product prepared in step b) are solubilized with 27 ml of 1,3-diaminopropane. Using the protocol described in step c) of Example 15, 5.5 g of product are isolated.

Mass Spectrum:

Mode ES⁻ m/z=1842.6 with z=4.

HPLC:

Symmetry® C18 column

water-TFA pH 2.9/CH₃CN

tr=12.8 min.

### EXAMPLE 38 (MC 798)

Compound of Formula: I₂ with

W₂: 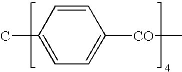
A₂ is II″ₐ₂ with x=2
G-NH, R and D′=D-H as defined in Example 37,
m₂=4.

By applying the protocol described in Example 33, starting with 0.5 g of the compound obtained in Example 37 step b) and 7.6 mg of tetrakis(4-carboxyphenyl)methane, 0.36 g of product is isolated.

Mass Spectrum:

Mode ES⁻ m/z=2491.8 with z=12.

HPLC:

PLRP-S® column

water-TFA pH 3.0/CH₃CN

tr=23-28 min.

### EXAMPLE 39

Compound of formula A1H in which A1 is the formula II′″1, with x=2

and R=

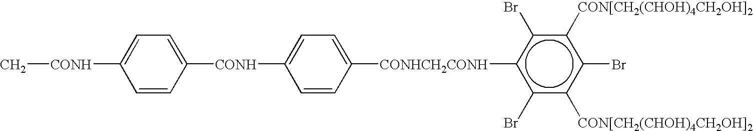

a) Coupling the Amine RNH₂

5 g of the compound obtained in step e) of Example 1 and 24.8 g of the amine RNH₂ are coupled in the presence of 6.25 g of 1-(3-dimethylaminopropyl)-3-ethylcarbodiimide hydrochloride (EDCl) and 0.34 g of (N-hydroxysuccinimidyl)-3-sulfonic acid (NHS) according to the protocol described in step a) of Example 6: so as to obtain 8.5 g of product.

HPLC:

Symmetry® C18 column

Water-TFA pH 2.9/CH₃CN

tr=21 min.

b) Deprotection of the Amino Group

8.5 g of the above compound are deprotected in the presence of 300 ml of CF₃COOH according to the protocol described in step b) of Example 6. After drying, 7.5 g of product are obtained.

HPLC:

Symmetry® C18 column

water-TFA pH 2.9/CH₃CN

tr=9 min.

### EXAMPLE 40 (MC808)

Compound of Formula: II′″₂ with x=2

-GNH— is —(—CH₂)₃—NH₋

R is

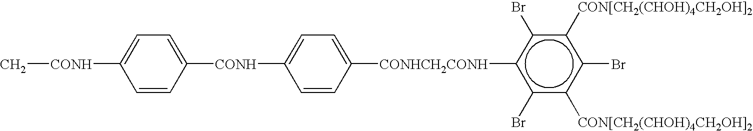

D′ is 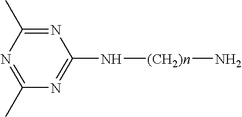
with n=3.

a) Condensation on the Triazine Ring

7.5 g of the compound obtained according to step b) of Example 39 are condensed on 0.135 g of 2,4,6-trichloro-1,3,5-triazine in the presence of 0.233 g of potassium carbonate according to the protocol described in step a) of Example 23. After isolation of the product, m=7 g are obtained.

Mass Spectrum:

Mode ES⁻ m/z=1693.7 with z=6

HPLC:

Symmetry® C18® column

Water/CH₃CN

tr 15 min (unresolved peak).

b) Introduction of the Diamine

The 7 g of the compound obtained in step a) are reacted in 60 ml of dimethyl sulfoxide, in the presence of 0.21 g of potassium carbonate and 0.75 ml of diaminopropane according to the protocol described in step b) of Example 23. 3.6 g of crystals are obtained.

Mass Spectrum:

Mode ES⁻ m/z=1700 with z=6

HPLC:

Lichrospher C18® column

water-TFA pH 3.3/CH₃CN

tr=19 min (unresolved peak).

### EXAMPLE 41 (MC809)

Compound of Formula: I₂ with

W₂: 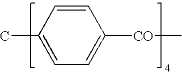
A₂ is II′″₂ with x=2
G-NH, R and D′=D-H as defined in Example 40
m₂=4.

By applying the protocol described in Example 36, starting with 1.6 g of the compound obtained in Example 40b) and 17 mg of tetrakis(4-carboxyphenyl)methane, 0.91 g of product is isolated.

Mass Spectrum:

Mode ES⁻ m/z=41247 with z=1

HPLC:

ZORBAX 300SB-CN® column

CH₃COONH₄ 100 mM/CH₃CN

tr=59 min (broad peak).

### EXAMPLE 42 (MC 802)

Compound of Formula: II′₂ with x=2

-GNH— is 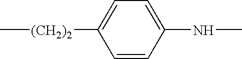
and R is 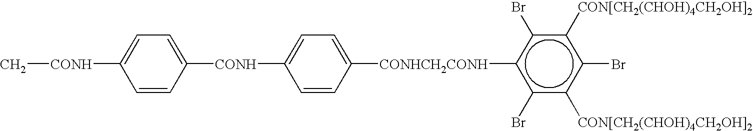
D′ is D-H: 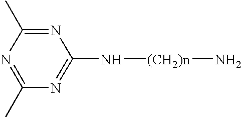
with n=3.

a) Coupling of the Amine R—NH₂

15.7 g of the amine of formula RNH₂, 0.2 g of sodium salt of (N-hydroxysuccinimidyl)-3-sulfonic acid (NHS) and 2.45 g of 1-(3-dimethylaminopropyl)-3-ethylcarbodiimide hydrochloride (EDCl) are introduced into the water-dioxane solution containing 2.9 g of the compound obtained in step a) of Example 15. The coupling is carried out according to the protocol described in step b) of Example 15, so as to obtain 13.15 g of product.

Mass Spectrum:

Mode ES⁻ m/z=1722.4 with z=6

HPLC:

Symmetry® C18 column

water-TFA pH 2.8/CH₃CN

tr=24 min (unresolved peak).

b) Introduction of the Diamine

Starting with 13.15 g of the compound obtained in step a), 8.65 g of powder are obtained according to the process described in step c) of Example 15 and after purification by preparative HPLC.

Mass Spectrum:

Mode ES⁻ m/z=1728.7 with z=6

HPLC:

Licrospher® RP18 column

Water/CH₃CN

tr=27 min (broad peak).

### EXAMPLE 43 (MC 803)

Compound of Formula: I₂ with

W₂: 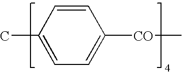
A₂ is II′₂ with x=2
G-NH, R and D′=D-H as defined in Example 42,
m₂=4.

By applying the protocol described in Example 33, starting with 1.5 g of the compound obtained in Example 42b) and 17 mg of tetrakis(4-carboxyphenyl)methane, 0.52 g of product is isolated.

Mass Spectrum:

Mode ES⁻ 41950 with z=1

SEC:

Columns OH Pack SB-HQ® SB-804, 2×SB-803, SB-802.5

Water-NaCl 0.1 6M/CH₃CN 70/30

tr=38 min (broad peak).

The results of relaxivity and the biological data illustrating the efficiency of the compounds according to the invention are now described.

The tables below give examples of results of relaxivity and of mass efficiency obtained, demonstrating the clearly greater mass efficiency me of the polymetallic compounds of RR derivatives (whether polymetallics of monomers or polymetallics of dimers), compared to the corresponding monomers: of the order of 80 to 100% for the family of compounds of the type P730, and of the order of 30 to 60% for the family of compounds of C-functionalized PCTA type. The Applicant has thus verified the validity of the results by comparing the polymetallics with monometallics of identical or virtually identical branch structure.

For example, for the polymetallics of monomers of the P730 family, the me in the field of 0.47 T (20 MHz) of the compound P799 is 7.3, compared with me=4.7 for the corresponding monomeric compound. P761.

For example, for the polymetallics of dimers of the P730 family, the me (0.47 T) of the compound P855 is 8.1, compared with me=4.67 for the corresponding monomeric compound P760.

TABLE 3
Efficiency of the polymetallic compounds of dimers (in the field of 0.47 T, 20 MHz)

| reference | polymetallic | | | | | relaxivity | | Molar mass | Gd | |
|---|---|---|---|---|---|---|---|---|---|---|
| monometallic | compound of | example | | molecular | number | r1 per Gd | r1/mol | efficiency me | content | |
| compound | dimers tested | number | Family | weight | of Gd | (mM⁻¹ · Gd · s⁻¹) | (mM⁻¹ s⁻¹) | (g⁻¹ s⁻¹) | (%) | Branch |
| | P 855 | 15 | P730 | 17292 | 4 | 35.1 | 140 | 8.1 | 3.64 | AAG1AA28 Br |
| | P 856 | 16 | P730 | 17381 | 4 | 34.8 | 139 | 8 | 3.62 | AAG1AA28 Br |
| | P 858 | 17 | P730 | 26068 | 6 | 39.9 | 239 | 9.2 | 3.62 | AAG1AA28 Br |
| | P 865 | 18 | P730 | 34850 | 8 | 37.9 | 303 | 8.7 | 3.61 | AAG1AA28 Br |
| | P 869 | 19 | P730 | 17290 | 4 | 33.6 | 134 | 7.8 | 3.64 | AAG1AA28 Br |
| | P 870 | 20 | P730 | 26203 | 6 | 38.7 | 232 | 8.9 | 3.6 | AAG1AA28 Br |
| | P 876 | 21 | P730 | 11821 | 3 | 31 | 93 | 7.9 | 3.99 | AAG1AA28 Br |
| | P 877 | 22 | P730 | 15402 | 3 | 40.8 | 122 | 7.9 | 3.06 | AAG1AA28 Br |
| | P 928 | 23 | P730 | 25835 | 8 | 25.7 | 206 | 8 | 4.87 | AAG1AA28 Br |
| | P 934 | 24 | P730 | 30534 | 8 | 32.6 | 261 | 8.5 | 4.12 | AAG1AA28 Br |
| P760 | | | P730 | 5292 | 1 | 24.7 | 24.7 | 4.67 | | AAG1AA28 Br |
| | P 867 | 25 | PCTA-C | 14833 | 4 | 44.3 | 177 | 11.9 | 4.24 | |
| | P 874 | 26 | PCTA-C | 10962 | 3 | 42.2 | 127 | 11.5 | 4.3 | |
| | P 878 | 27 | PCTA-C | 22462 | 6 | 48.9 | 293 | 13.1 | 4.2 | |
| | P 943 | 28 | PCTA-C | 29838 | 8 | 45.5 | 364 | 12.2 | 4.22 | |
| | P 949 | 29 | PCTA-N | 22306 | 8 | 37.3 | 298 | 13.4 | 5.64 | |
| P846 | | | PCTA | 3545 | 1 | 27.9 | 27.9 | 7.9 | 4.44 | |

The following are specified:

TABLE 4
Formula of P846

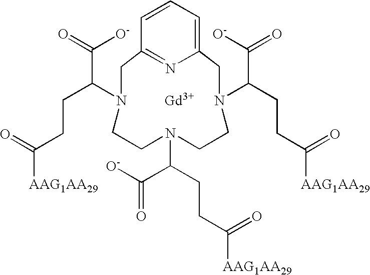

Formula of P760

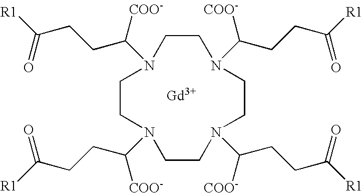

with R1 = AAG1AA28

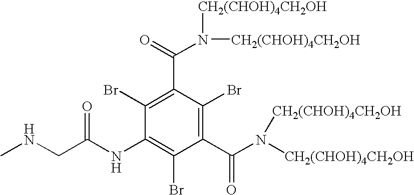

Efficiency of the polymetallic compounds of monomers

| | Polymetallic | | | | | | | | | |
|---|---|---|---|---|---|---|---|---|---|---|
| Reference | compound of | | | | | Relaxivity | | Molar mass | | |
| monometallic | monomers | Example | | Molecular | Number | r1 | | efficiency me | Gd | |
| compound | tested | number | Family | weight | of Gd | per Gd | r1/mol | r1 | content | Branch |
| | P 799 | 10 | P730 | 14027 | 3 | 34.1 | 102.3 | 7.3 | 3.36 | AAG1 AA28 I |
| P 761 | | | P730 | 5856 | 1 | 27.6 | 27.6 | 4.7 | 2.69 | AAG1 AA28 I |
| | P 876 | 12 | P730 | 11821 | 3 | 31 | 93.0 | 7.9 | 3.99 | monoarom_G1AA 29Br |
| P 796 | | | P730 | 5996.7 | 1 | 23.3 | 23.3 | 3.9 | 2.62 | monoarom_G1AA 28Br |
| | P 877 | Ex 11 | P730 | 15402 | 3 | 40.8 | 122.4 | 7.9 | 3.06 | Bisarom_G1AA28 Br |
| P 792 | | | P730 | 6473.2 | 1 | 42 | 42.0 | 6.5 | 2.43 | Bisarom_G1AA28 Br |
| | P 874 | Ex 8 | PCTA C | 10962 | 3 | 42.2 | 126.6 | 11.5 | 4.30 | AAG1 AA29 Br |
| | P 867 | Ex 7 | PCTA C | 14833 | 4 | 44.3 | 177.2 | 11.9 | 4.24 | AAG1 AA29 Br |
| | P 878 | Ex 9 | PCTA C | 22462 | 6 | 48.9 | 293.4 | 13.1 | 4.20 | AAG1 AA29 Br |
| P 846 | | | PCTA | 3545 | 1 | 27.9 | 27.9 | 7.9 | 4.44 | AAG1 AA29 Br |

The data were measured at a frequency of 20 MHz, which constitutes a common frequency for the diagnostic indications in question. Favorable results were also obtained at higher frequencies, for example 40 MHz.

The Applicant has thus succeeded in obtaining oligomers
which relax at more than 75 mM⁻¹s⁻¹Gd⁻¹, from compounds (monomers or dimers) of RR type which relax individually at more than 25 mM⁻¹s⁻¹Gd⁻¹, and with a Gd mass fraction that is less by about 4 to 6%, i.e. of the order of at least-three times less than that of known dendrimer-type compounds;

oligomers
which relax at more than 75 mM⁻¹s⁻¹, from compounds which relax individually at more than 25 mM⁻¹s⁻¹Gd⁻¹,
the charge of which can be modulated, and the external covering of which can be modulated (hydrophilicity).

Experimental proof of the very advantageous diagnostic use of various polymetallics of dimers according to the invention is now described.

The inventors have demonstrated very advantageous BPA properties, in particular in rats and in humans. With a high molecular weight, the compounds obtained undergo very little extravasation. The compounds exhibit a very advantageous pharmacokinetic profile of BPA type:
a very prolonged intravascular circulation (circulating fraction at 5 minutes C5/C0 greater than 30%, and typically from 40 to 60%), two to three times greater than that of RC BPAs described, for. example, in patent EP 922 700, persisting over long periods, which is an advantage in MRI examination (C15/C0=20 to 35%); in the knowledge that the concentration C0 represents the ratio between the dose injected and the theoretical plasma volume of 25 ml/kg in rats; which confirms the desired BPA type behavior;
associated with very low tissue extravasation (distribution volume at equilibrium Vdₛₛ=30 to 40 ml/kg), which reflects vascular containment and, for some, also a plasma clearance which is clearly decreased (1.2 to 1.8 ml/min/kg, compared with 4 to 6 for the BPAs of the prior art) which reflects behavior of SCBPA type.

The compounds according to the invention are very useful in diagnostic imaging, in particular in tumor characterization. Conclusive trials were obtained, for example, on human breast tumors and in rats.

Differentiation between benign and malignant tumors was clearly improved, for example, by means of compounds exemplified above which exhibit an SCBPA pharmacokinetic profile.

In rats, the inventors obtained in particular the following results. The controls. were Dota-dysprosium, and the compound P792 (described in granted patent EP 922 700), which is a Gd-based monometallic product the pharmacokinetic profile of which is that of an RCBPA. The measurements are carried out 30 and 60 minutes post-injection.

Doses of cancerigenic agent ENU of 45 mg/kg, to induce mainly fibroadenomas (benign tumors), and of 180 mg/kg, to induce mainly adenocarcinomas (malignant tumors) were tested. The concentrations of contrast products measured in the adenocarcinomas are clearly greater than those recorded in the muscle (tumor/muscle enhancement) and then those obtained in the fibroadenomas (malignant tumor/benign tumor enhancement). A predominant accumulation of SCBPA polymetallic (0.5 to 0.6% ID/g) is found compared to the Dota-Dy (0.15% ID/g). In addition, for the Dota-Dy, whether the fibroadenomas or the adenocarcinomas are considered, the tumor/muscle concentration ratio is in the region of 4. In the case of polymetallics according to the invention exemplified above, the ratios are different: the ratio is 4 to 5 for the fibroadenomas against 8 to 9 for the adenocarcinomas, which makes it possible to differentiate the benign and malignant tumors, 1 hour after injection. For comparison, Daldrup-Link et al. have shown that Gadomer-17 does not allow such a differentiation in MRI in this model [Daldrup-Link H. E. et al., Comparison of Gadomer-17 and gadopentetate dimeglumine for differentiation of benign from malignant breast tumors with MR imaging. Acad. Radilo. 2000. 7: 934-944.]. This inefficiency of Gadomer-17 is probably explained by its pharmacokinetic characteristics: specifically, this product has a C₅/C₀ ratio of 21% (instead of 60% for polymetallics obtained by the inventors), a plasma half-life of 37 min (instead of 20 min for polymetallics obtained by the inventors), a distribution volume of 130 ml/kg (instead of 40 ml/kg for polymetallics obtained by the inventors) and a total plasma clearance of 8.1 ml/min/kg (instead of 1.5 ml/min/kg for polymetallics obtained by the inventors) [Misselwitz B. et al. Pharmacokinetics of Gadomer-17, a new dendritic magnetic resonance contrast agent MAGMA. 2001. 12: 128-134.]. Consequently, despite a comparable molecular weight, Gadomer-17 rather has a pharmacokinetic behavior which is not suitable for demonstrating differences in vascular permeability between the two types of tumors, unlike polymetallic compounds according to the invention. Polymetallics obtained by the inventors not only enable, over the selected period of time, a significant enhancement in the signal for tumors compared to the muscle, but also make it possible to differentiate tumors, the concentrations contained in adenocarcinomas being clearly greater than those found in fibroadenomas.

1-33. (canceled)

34. A compound of general formula below:
W-(A)ₘ (E)
in which:
W is a central NUCLEUS
A represents [(D)_{q}-(I_{a,b,c,d,e,f})ᵣ];
with:
q=0 or q=1
r=1 when q=0, or r is 2 or 3 when q=1
m is between 3 and 6 when q=0 and m is between 2 and 4 when q=1
D is a polyfunctional molecule capable of linking to the central NUCLEUS W by means of a linker 2 and to at least two metal chelates by means of linkers 1
I_{a,b,c,d,e,f} signifies Iₐ or I_{b} or I_{c} or I_{d} or Iₑ or I_{f}, representing the restricted-rotation derivatives
Iₐ, I_{b}, I_{c} having the meanings: 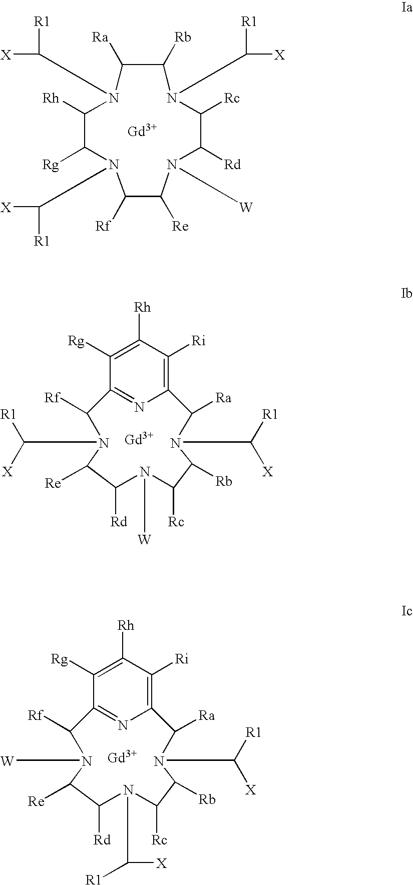
where:
X represents CO₂R′a, CONR′bR′c or P(R′d)O₂H, with:
R′a, R′b and R′c representing H or optionally hydroxylated (C₁-C₈)alkyl;
R′d represents OH, (C₁-C₈)alkyl or (C₁-C₈)alkoxy, (C₁-C₈)arylalkyl or (C₁-C₈)alkoxyalkyl;
R1 represents a hydrophilic group of molecular mass greater than 300 and less than 3000, comprising at least three oxygen atoms, selected from the following groups:
polyoxy (C₂-C₃)alkylene, in particular polyethylene glycol and C₁ to C₃ monoethers and monoesters thereof
polyhydroxyalkyl
polyol
(R₂ g)ₑ[(R₂ g)ᵢR₃]ₕ where:
h=1 or 2; i=0, 1 or 2; e=1 to 5
R₂ represents (the R₂ being identical or different):
nothing, an alkylene, an alkoxyalkylene, a polyalkoxyalkylene;
a phenylene, or a saturated or unsaturated heterocyclic residue optionally substituted with OH, Cl, Br, I, (C₁-C₈)alkyl, (C₁-C₈)alkyloxy, NO2, NR_{X}R_{Y}, NR_{X}COR_{Y}, CONR_{X}R_{Y} or COOR_{X}, R_{X} and R_{Y} being H or (C₁-C₈)alkyl, and the linear, branched or cyclic C₁ to C₁₄ alkyl, alkylene and alkoxy groups possibly being hydroxylated;
g represents (the g being identical or different): nothing or a function O, CO, OCO, COO, SO3, OSO2, CONR′, NR′CO, NR′COO, OCONR′, NR′, NR′CS, CSNR′, SO2NR′, NR′SO2, NR′CSO, OCSNR′, NR′CSNR′, P(O)(OH)NR′ or NR′P(O)—(OH), in which R′ is H (C₁-C₈) or R₃;
R₃ represents alkyl, phenyl, alkyl substituted or interrupted with one or more phenyl groups, or alkyleneoxy; amino or amido, unsubstituted or substituted with alkyl optionally substituted or interrupted with one of the above groups; it being possible for the phenyl, phenylene and heterocyclic groups to be substituted with OH, Cl, Br, I, (C₁-C₈)alkyl, (C₁-C₈)alkyloxy, NO2, NR_{X}R_{Y}, NR_{X}COR_{Y}, CONR_{X}R_{Y} or COOR_{X}, R_{X} and R_{Y} being H or (C₁-C₈)alkyl, and the linear, branched or cyclic C₁ to C₁₄ alkyl, alkylene and alkoxy groups possibly being hydroxylated;
Rₐ to Rᵢ independently represent H, alkyl, hydroxyalkyl, alkylphenyl or cycloalkyl.
U is a group —CX R4-linker 1, CHR4CON-linker1 or CHR4-CHR50H-linker1
R4 and R5 independently represent H, alkyl or hydroxyalkyl.
X having the meaning above.
I_{d}, Iₑ, I_{f} having the meanings: 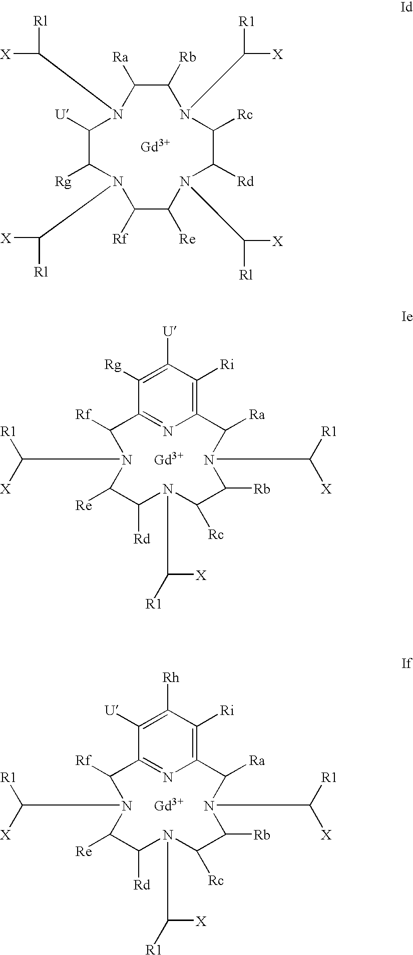
X, R1, and Ra to Ri having the same meaning as above.
U′ is linker 1;
and also the pharmacologically acceptable salts of the compounds of formula (E) with inorganic or organic acids or bases.

35. The compound as claimed in claim 34, in which:
D is an aromatic backbone polyfunctionalized with carboxylate and/or amino groups, 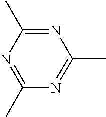
linker 1 and linker 2 are chosen from a) and b),
a) (CH₂)₂-φ-NH₂, (CH₂)₃—NH₂, NH—(CH₂)₂—NH and NH—(CH₂)₃—NH,
a) P1-I-P2, which may be identical or different, P1 and P2 being chosen from OH, SH, NH₂, nothing, CO₂H, NCS, NCO, SO₃H,
With I=alkylene, alkoxyalkylene, polyalkoxyalkylene, alkylene interrupted with phenylene, alkylidene or acylidene;
W is the radical of an organic molecule carrying m carbonyl groups which form carboxamido groups with A; or W is a group:

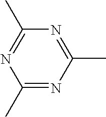

36. The compound as claimed in claim 35, in which D is of 1,3,5-triazine type, of formula:

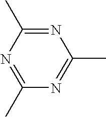

37. The compound as claimed in claim 34, in which R1 represents (CH₂) xCONHR with x=1, 2 or 3, and R is a hydrophilic group of molecular weight greater than 300, chosen from:
1) a group: 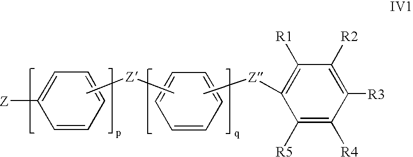
and Z is a bond, CH₂, CH₂CONH or (CH₂)₂NHCO
Z′ is a bond, O, S, NQ, CH₂, CO, CONQ, NQCO, NQ-CONQ or CONQCH₂CONQ,
Z″ is a bond, CONQ, NQCO or CONQCH₂CONQ
p and q are integers the sum of which is 0 to 3;
R₁, R₂, R₃, R₄ and R₅ represent:
either, independently of one another, H, Br, Cl, I, CONQ₁Q₂ or NQ₁COQ₂ with Q₁ and Q₂, which may be identical or different, being H or a group (C₁-C₈)alkyl that is mono- or polyhydroxylated or optionally interrupted with one or more oxygen atoms, and at least one and at most two of R₁ to R₅ being CONQ₁Q₂ or NQ₁COQ₂;
or R₂ and R₄ represent 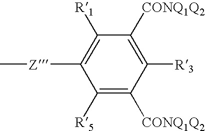
and R₁, R′₁, R₃, R′₃, R₅ and R′₅, which may be identical or different, represent H, Br, Cl or I, Q₁ and Q₂ have the same meaning as above and Z′″ is a group chosen from CONQ, CONQCH₂CONQ, CONQCH₂, NQCONQ and CONQ(CH₂)₂NQCO, and Q is H or optionally hydroxylated (C₁-C₄)alkyl, it being possible for the alkyl groups to be linear or branched;
2) a “flash” branch 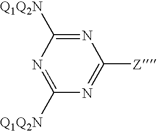
with Z″″ being NQCH₂ⱼ(CH₂OCH₂)ᵢ(CH₂)ⱼNH₂, with i=2 to 6 and j=1 to 6.

38. The compound as claimed in claim 34, of formula:
W1-(A1)ₘ₁ (I1) PolyM RR
in which:
m1 is 3,4,5 or 6;
W1 is the radical of an organic molecule carrying m1 carbonyl groups which form carboxamido groups with A1;
or W1 is a group: 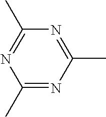
A1 represents the group: 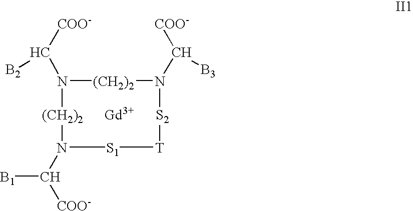
in which:
—S₁-T-S₂— is
either 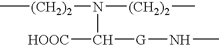
where S₁=S₂=(CH₂)₂
with all three of B₁, B₂ and B₃ representing (CH₂)ₓCONHR with x=1, 2 or 3
or 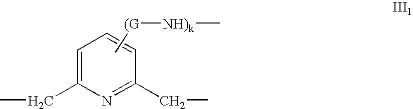
with k=0 and S₁=S2=CH₂
one of B1, B2 and B3 representing G-NH, and the others representing (CH₂)ₓCONHR
or 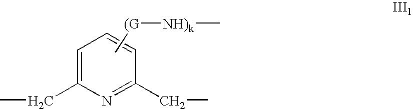
with k=1
all three of B₁, B₂ and B₃ representing (CH₂)ₓCONHR with x=1, 2 or 3 and GNH chosen from:
the groups —(CH₂)ₙ—NH— with n=1 to 4,
or 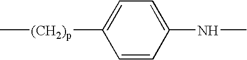
with p=0 to 3;
it being specified that:
R represents a group: 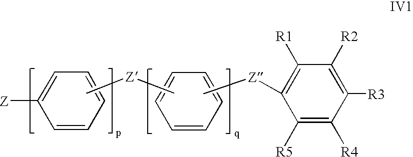
and Z is a bond, CH₂, CH₂CONH or (CH₂)₂NHCO
Z′ is a bond, O, S, NQ, CH₂, CO, CONQ, NQCO, NQ-CONQ or CONQCH₂CONQ,
Z″ is a bond, CONQ, NQCO or CONQCH₂CONQ
p and q are integers the sum of which is 0 to 3;
R₁, R₂, R₃, R₄ and R₅ represent:
either, independently of one another, H, Br, Cl, I, CONQ₁Q₂ or NQ₁COQ₂ with Q₁ and Q₂ which may be identical or different, being H or a group (C₁-C₈)alkyl which is mono- or polyhydroxylated or optionally interrupted with one or more oxygen atoms, and at least one and no more than two of R₁ to R₅ being CONQ₁Q₂ or NQ₁COQ₂;
or R₂ and R₄ represent 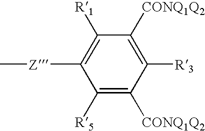
and R₁, R′₁, R₃, R′₃, R₅ and R′₅, which may be identical or different, represent H, Br, Cl or I, Q₁ and Q₂ have the same meaning as above and Z′″ is a group chosen from CONQ, CONQCH₂CONQ, CONQCH₂, NQCONQ and CONQ(CH₂)₂NQCO, and Q is H or optionally hydroxylated (C₁-C₄)alkyl, it being possible for the alkyl groups to be linear or branched;
or a FLASH group of formula: 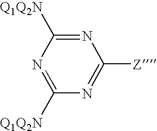
with Z″″ being NQCH₂(CH₂OCH₂)ᵢ(CH₂)ⱼNH₂, with i=2 to 6 and j=1 to 6;
and also the pharmacologically acceptable salts of the compounds of formula I1 with inorganic or organic acids or bases.

39. The compound as claimed in claim 34, of formula:
W2-(A2)ₘ₂ (I2) PolyD RR
in which:
m2 is 2, 3 or 4;
W2 is the radical of an organic molecule carrying m2 carbonyl groups which form carboxamido groups with A2;
or W2 is a group: 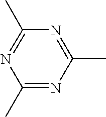
A2 represents 1) the group 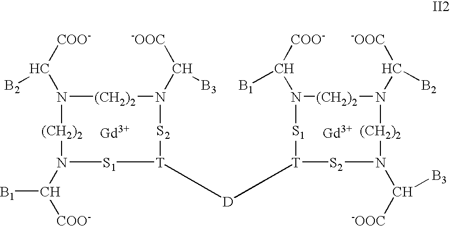
in which
—S₁-T-S₂— is 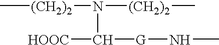
where S₁=S₂=(CH₂)₂
all three of B₁, B₂ and B₃ representing (CH₂)ₓCONHR with x=1, 2 or 3
or A2 represents 2) 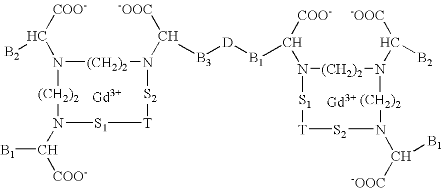
IIa2 (compound known as N-functionalized PCTA)
Or IIb2 (compound referred to as N-functionalized PCTA and positional isomer of IIb2) 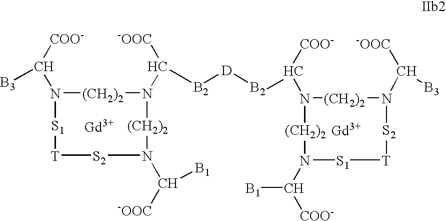
in both of which S₁-T-S₂— is: 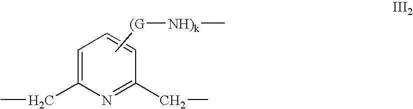
with k=0 and S₁=S₂=CH₂;
B₃ representing G-NH, and B1 and B2 representing (CH₂)ₓCONHR for IIa2 with x=1, 2 or 3,
B₂ representing G-NH, and B1 and B3 representing (CH₂)ₓCONHR for IIb2;
or A2 represents 3) 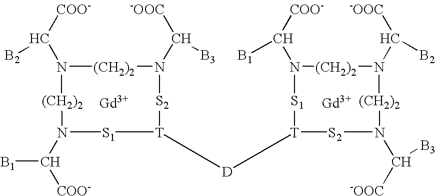
IIc2 (compound referred to as C-functionalized PCTA)
when S₁-T-S₂— is: 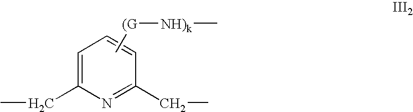
with k=1 and S₁=S2=CH₂;
all three of B₁, B₂ and B₃ represent (CH₂),CONHR with x=1, 2 or 3 for IIc2, in the knowledge that, for II2, IIa2, IIb2 and IIc2,
GNH is chosen from the groups —(CH₂)ₙ—NH— with n=1 to 4,
or 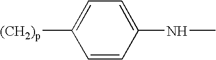
with p=0 to 3;
D being (Div−linker 2) and being a divider, with Div being of 1,3,5-triazine type,:
R represents a group: 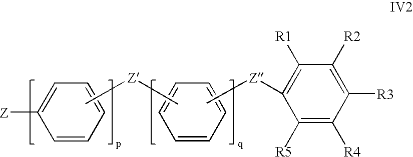
and Z is a bond, CH₂, CH₂CONH or (CH₂)₂NHCO
Z′ is a bond, O, S, NQ, CH₂, CO, CONQ, NQCO, NQ-CONQ or CONQCH₂CONQ,
Z″ is a bond, CONQ, NQCO or CONQCH₂CONQ
p and q are integers the sum of which is 0 to 3;
R₁, R₂, R₃, R₄ and R₅ represent:
either, independently of one another, H, Br, Cl, I, CONQ₁Q₂ or NQ₁COQ2 with Q₁ and Q₂ which may be identical or different, being H or a group (C₁-C₈)alkyl which is mono- or polyhydroxylated or optionally interrupted with one or more oxygen atoms, and at least one and no more than two of R₁ to R₅ being CONQ₁Q₂ or NQ₁COQ₂;
or R₂ and R₄ represent 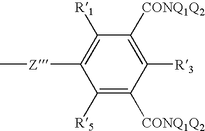
and R₁, R′₁, R₃, R′₃, R₅ and R′₅, which may be identical or different, represent H, Br, Cl or I, Q₁ and Q₂ have the same meaning as above and Z″″ is a group chosen from CONQ, CONQCH₂CONQ, CONQCH₂, NQCONQ and CONQ(CH₂)₂NQCO, and Q is H or optionally hydroxylated (C₁-C₄)alkyl, it being possible for the alkyl groups to be linear or branched;
or a FLASH group of formula: 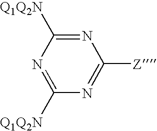
with Z″″ being NQCH2(CH2OCH2)i(CH2)jNH2, with i=2 to 6 and j=1 to 6; and also the pharmacologically acceptable salts of the compounds of formula I1 with inorganic or organic acids or bases.

40. The compound as claimed in claim 39, in which D is:

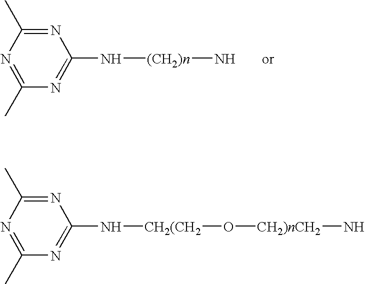

41. The compound as claimed in claim 34, in which, respectively, W for the compounds of formula (E), W1 for the compounds of formula I1 and W2 for the compounds of formula I1, are chosen from the groups: 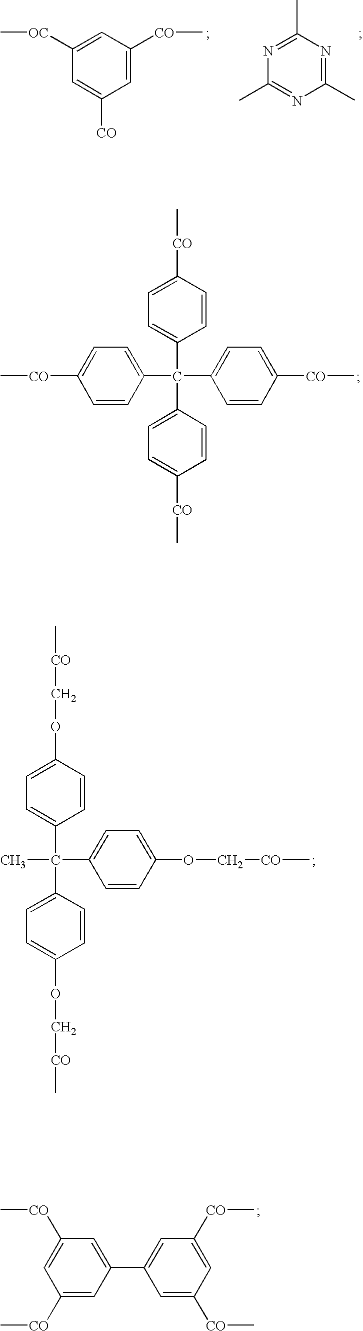 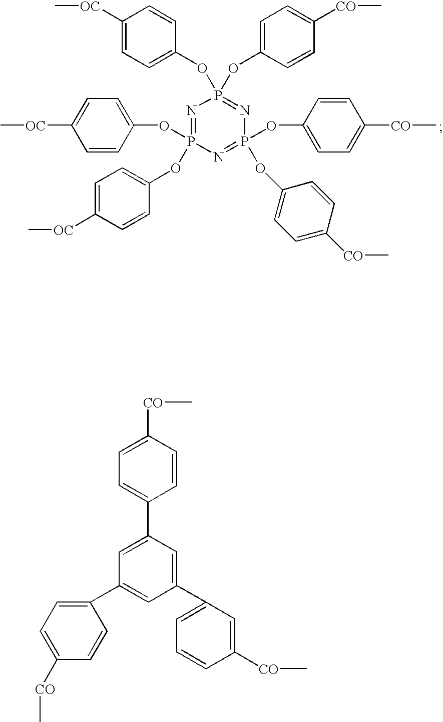
and m is equal to the number of free bonds of W.

42. The compound as claimed in claim 34, in which, respectively, W for the compounds of formula (E), W1 for the compounds of formula I1, and W2 for the compounds of formula I2, are:

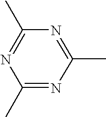

43. The compound as claimed in claim 34, in which, respectively, W for the compounds of formula (E), W1 for the compounds of formula I1, and W2 for the compounds of formula I2, are:

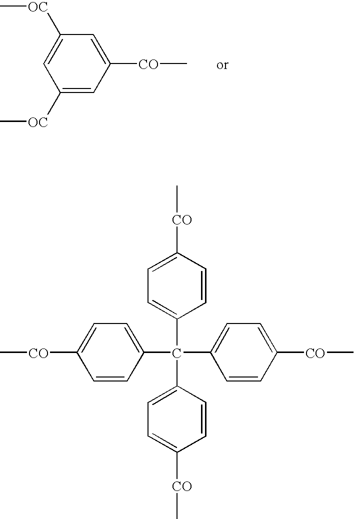

44. The compound as claimed in claim 34, in which, respectively, W for the compounds of formula (E), W1 for the compounds of formula I1, and W2 for the compounds of formula I2, are:

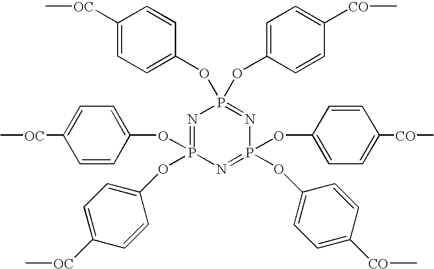

45. The compound as claimed in claim 34, in which x is 2.

46. The compound as claimed in claim 38, of formula I1, in which —S₁-T-S₂— represents: 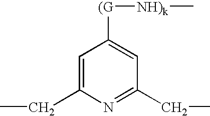
with S₁=S₂=CH₂.

47. The compound as claimed in claim 38 of formula I1, in which k is 1 and G is —(CH₂)₃—.

48. The compound as claimed in claim 38, of formula I1, in which k is 0 and B₂ or B₃ represents —(—CH₂)₃NH— or

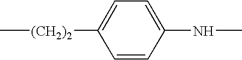

49. The compound as claimed in claim 38, of formula I1 or I2, in which —S₁-T-S₂— represents: 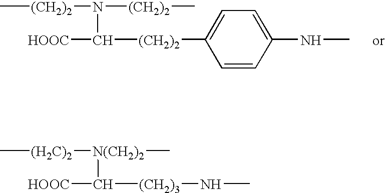
with S₁=S₂=(CH₂)₂.

50. The compound as claimed in claim 34, for which B₁, B₂ and B₃, when they do not represent -G-NH, represent —(CH₂)₂CONHR, with, in R, p=q=0 and Z being —CH₂CONH.

51. The compound as claimed in claim 50, for which R represents: 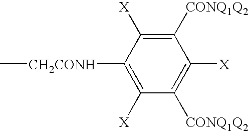
and the X are identical and represent Br or I, while Q₁ and Q₂, which may be identical or different, are mono- or polyhydroxylated (C₁-C₈)alkyl groups, such that each CONQ₁Q₂ contains from 4 to 10 hydroxyls in total.

52. The compound as claimed in claim 50, for which R represents: 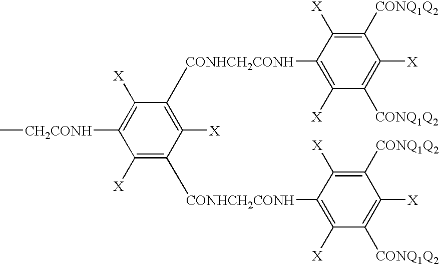
and the X, which are identical, are Br or I, and Q₁ and Q₂, which may be identical or different, are mono- or polyhydroxylated (C₁-C₈)alkyl groups, such that each group CONQ₁Q₂ contains from 4 to 10 hydroxyls in total.

53. The compound as claimed in claim 34, for which R represents: 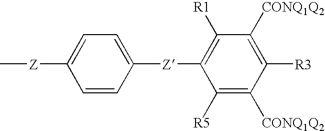
Z is CH₂ or CH₂CONH, Z′ is CONH or CONHCH₂CONH, R₁, R₃ and R₅, which may be identical, are Br or I, Q₁ and Q₂, which may be identical or different, being mono- or polyhydroxylated (C₁-C₈)alkyl groups, such that each group CONQ₁Q₂ contains from 4 to 10 hydroxyls in total.

54. The compound as claimed in claim 34, for which R represents: 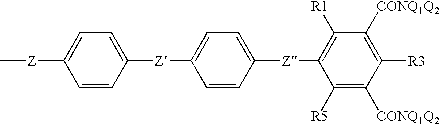
Z is CH₂CONH, Z′ is CONH, Z″ is CONHCH₂CONH, and R₁, R₃ and R₅, which are identical, are Br or, and Q₁ and Q₂, which may be identical or different, are monohydroxylated or polyhydroxylated (C₁-C₈)alkyl groups, such that each group CONQ₁Q₂ contains from 4 to 10 hydroxyls in total.

55. The compound as claimed in claim 34, for which R represents: 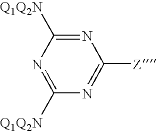
with Z″″ being NQ(CH₂)ⱼ(CH₂OCH₂)ᵢ(CH₂)ⱼNH₂, with i=2 to 6 and j=1 to 6.

56. The compound as claimed in claim 55, in which R represents: 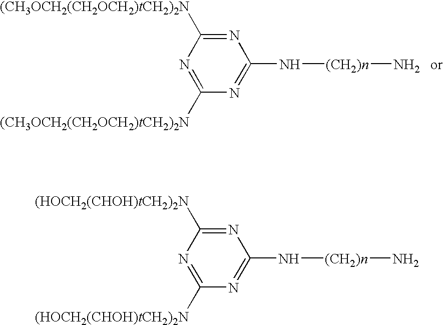
with t=1, 2, 3 or 4 and n=2 to 6.

57. The compound as claimed in claim 34, in which Q₁ represents CH₂CHOHCH₂OH or CH₂(CHOH)₄CH₂OH and Q₂ represents CH₂(CHOH)₄CH₂OH.

58. A compound of formula 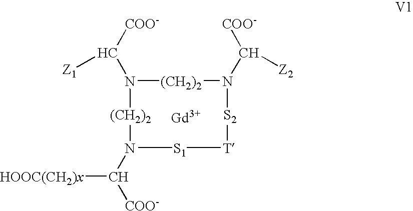
in which x=1, 2 or 3 and —S₁-T′-S₂— is:
1) 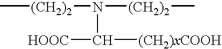
with S₁=S2=(CH₂)₂
or
2) 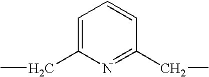
with S₁=S2=CH₂
and one of the groups Z₁ or Z₂ is chosen from the groups —(CH₂)₃NH₂ or 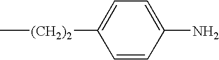
and the other of Z₁ or Z₂ is (CH₂)ₓCOOH;
and also its salts with an alkaline base.

59. The compound as claimed in claim 58, of formula V1, in which x=2.

60. The compound as claimed in claim 58 of formula: 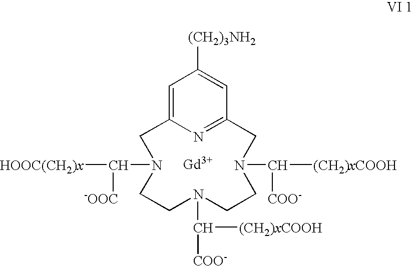
in which x=1, 2 or 3
and its salts with an alkaline base.

61. A compound of formula: 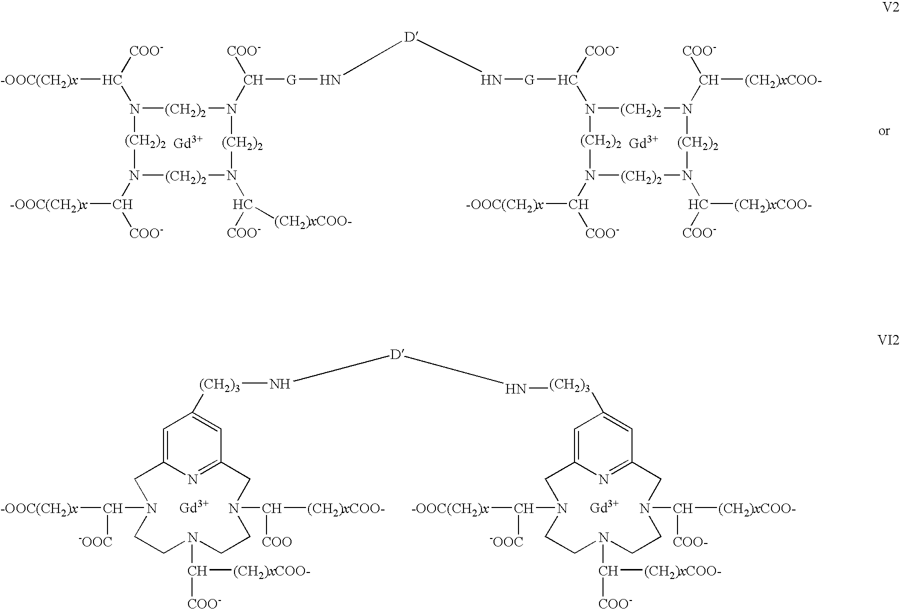
in which x=1, 2 or 3
D′=D-H wherein D is (Div−linker 2) as defined in claim 34, linker 2 being chosen from:
a) (CH₂)₂-φ-NH₂, (CH₂)₃—NH₂, NH—(CH₂)₂—NH, NH—(CH₂)₃—NH, NH₂—(CH₂)₂—O(CH₂)₂—O(CH₂)₂—NH₂,
b) P1-I-P2, which may be identical or different, P1 and P2 being chosen from OH, SH, NH₂, H, CO₂H, NCS, NCO, SO₃H,
With I=alkylene, cycloalkylene, alkoxyalkylene, polyalkoxyalkylene, alkylene interrupted with phenylene, alkylidene, acylidene and its salts with an alkaline base.

62. The compound as claimed in claim 61, in which Div is of 1,3,5-triazine type.

63. The compound as claimed in claim 62, in which D is: 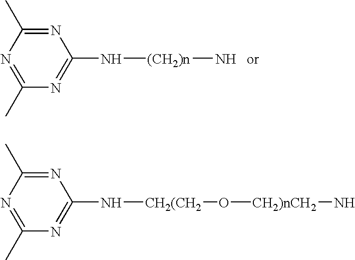
with n=2 to 6.

64. A contrast product for magnetic resonance imaging, which comprises a compound as claimed in claim 34, optionally combined with a pharmacologically acceptable carrier.

65. The contrast product as claimed in claim 64, provided in the form of an injectable sterile solution.

66. The contrast product as claimed in claim 64, for its use in the diagnosis of a cardiovascular, cancerous or inflammatory pathology.

67. A use of a contrast product as claimed in claim 64, for preparing a composition intended for the diagnosis of a cardiovascular, cancerous or inflammatory pathology.

68. The compound as claimed in claim 34, having a molar mass efficiency of between 7 and 15.

69. A screening method, consisting in selecting the compounds of formula (E) as claimed in claim 34, which are effective from a diagnostic point of view, said method comprising:
preparing a candidate polymetallic compound of formula (E),
using the candidate compound in a test protocol suitable for a diagnostic indication,
selecting the candidates exhibiting a mass efficiency of at least 30%, greater than that of the corresponding non-polymetallic chelate.
